(19) United States
(12) Patent Application Publication
(10) Pub. No.: US 2025/0138016 A1
(43) Pub. Date: May 1, 2025

(54) METHODS FOR THE DETECTION AND TREATMENT OF OVARIAN CANCER

(71) Applicant: Board of Regents, The University of Texas System, Austin, TX (US)

(72) Inventors: Samir HANASH, Houston, TX (US); Johannes Francois FAHRMANN, Houston, TX (US); Ehsan IRAJIZAD, Houston, TX (US)

(21) Appl. No.: 19/012,572

(22) Filed: Jan. 7, 2025

Related U.S. Application Data
(63) Continuation of application No. PCT/US2023/070467, filed on Jul. 19, 2023.
(60) Provisional application No. 63/369,027, filed on Jul. 21, 2022.

(51) Int. Cl. G01N 33/574; A61K 45/06; G01N 33/68

(52) U.S. Cl. CPC G01N 33/57449; A61K 45/06; G01N 33/57492; G01N 33/57496; G01N 33/6893; G01N 2800/34; G01N 2800/50

(57) ABSTRACT

A novel 7-marker metabolite panel (7MetP) comprising or consisting of diacetylspermine, diacetylspermidine, N-(3-acetamidopropyl)pyrrolidin-2-one, N-acetylneuraminate, N-acetyl-mannosamine, N-acetyl-lactosamine, and hydroxyisobutyric acid is described.

This application is a bypass continuation of International Patent Application No. PCT/US2023/070467, filed Jul. 19, 2023, which claims the benefit of priority of U.S. Provisional Application No. 63/369,027, filed Jul. 21, 2022, the disclosures of which are hereby incorporated by reference as if written herein in their entireties.

This invention was made with government support under grant numbers CA200462, and CA217685 awarded by the National Institutes of Health. The government has certain rights in the invention.

Disclosed herein are methods and related kits for detection of ovarian cancer. Also provided are methods for treating a patient susceptible, or suspected of being susceptible, to ovarian cancer.

Ovarian cysts and pelvic masses are found to occur in some 17% of women that undergo transvaginal sonograms (TVS). However, most such masses are benign and only a small percentage of these women will be diagnosed with ovarian cancer. Currently, neither TVS nor cancer antigen 125 (CA125) alone or in combination yield sufficient sensitivity and specificity to distinguish benign from malignant ovarian cysts. The high false positive rates lead to increased patient anxiety and unnecessary surgical procedures that are associated with significant morbidity.

Two risk assessment algorithms, the Risk of Ovarian Malignancy Algorithm (ROMA) and the risk of ovarian cancer algorithm (OVERA), were developed to estimate the probability of a woman with a pelvic mass harboring a malignancy, and to determine whether a patient should be referred to a general gynecologist if the mass is likely to be benign or a gynecologic oncologist if the mass is likely to be malignant. A gynecologic oncologist has specialized training to dissect nodes, remove the omentum, and to remove as much cancer as possible from the surface of the bowel if extensive disease is found. Although the OVERA and ROMA algorithms offer high sensitivity, they are limited by sub-optimal specificity which can result in the aforementioned high false-positive rates. A test that offers high sensitivity and specificity for identifying individuals at high risk of harboring malignant ovarian cysts has potential to better inform clinical decision making and improve patient outcomes.

Accordingly, a need exists for a method or test to aid the detection of ovarian cancer. Perturbed cellular metabolism is a hallmark of cancer. Several lines of evidence indicate that cellular and systemic metabolic adaptations occur from the earliest phases of cancer development, suggesting that metabolites may serve as cancer biomarkers. A novel seven-marker metabolite panel comprising or consisting of diacetylspermine, diacetylspermidine, N-(3-acetamidopropyl)pyrrolidin-2-one, N-acetylneuraminate, N-acetyl-mannosamine, N-acetyl-lactosamine, and hydroxyisobutyric acid has been discovered through a deep learning approach to metabolic profiles of sera that distinguishes early-stage ovarian cancers from benign disease. In combination with the ROMA algorithm, which utilizes the biomarkers Human epididymal protein 4 (HE4) and Mucin 16 (CA125), this model demonstrates superior ovarian cancer risk prediction among women with ovarian cysts compared to ROMA alone.

### SUMMARY

Provided herein is a method of treatment of ovarian cancer in a patient having elevated levels of diacetylspermine (DAS), diacetylspermidine (DiAcSpd), N-(3-acetamidopropyl)pyrrolidin-2-one (N3AP), N-acetylneuraminate (NANA), N-acetyl-mannosamine (NAcMan), N-acetyl-lactosamine (NAcLac), and hydroxyisobutyric acid (HBA), and optionally, elevated levels of Human epididymal protein 4 (HE4) and Mucin 16 (CA125), wherein the elevated levels classify the patient as having ovarian cancer, comprising administering a therapeutically effective amount of a treatment for ovarian cancer to the patient.

Also provided herein is a method of treatment of ovarian cancer, comprising:
a) identifying a patient having elevated levels of diacetylspermine, diacetylspermidine, N-(3-acetamidopropyl)pyrrolidin-2-one, N-acetylneuraminate, N-acetyl-mannosamine, N-acetyl-lactosamine, and hydroxyisobutyric acid, and optionally, elevated levels of Human epididymal protein 4 (HE4) and Mucin 16 (CA125), wherein the elevated levels classify the patient as having ovarian cancer; and
b) administering a therapeutically effective amount of a treatment for ovarian cancer to the patient.

Also provided herein is a method of distinguishing ovarian cancer from a benign pelvic mass (BPM) in a subject, comprising, in a biological sample obtained from the subject:
a) measuring the levels of diacetylspermine, diacetylspermidine, N-(3-acetamidopropyl)pyrrolidin-2-one, N-acetylneuraminate, N-acetyl-mannosamine, N-acetyl-lactosamine, and hydroxyisobutyric acid, and optionally, the levels of Human epididymal protein 4 (HE4) and Mucin 16 (CA125), in the biological sample; and
b) classifying the subject as having either ovarian cancer or a BPM based on said measured levels.

Also provided herein is a method of determining the risk of a subject for harboring ovarian cancer, comprising, in a biological sample obtained from the subject:
a) measuring the levels of diacetylspermine, diacetylspermidine, N-(3-acetamidopropyl)pyrrolidin-2-one, N-acetylneuraminate, N-acetyl-mannosamine, N-acetyl-lactosamine, and hydroxyisobutyric acid, and optionally, the levels of Human epididymal protein 4 (HE4) and Mucin 16 (CA125), in the biological sample; and
b) classifying the subject as being at risk of harboring ovarian cancer or not being at risk of harboring ovarian cancer based on said measured levels.

Also provided herein is a method of producing a risk profile of a subject for harboring ovarian cancer, comprising, in a biological sample obtained from the subject:
a) measuring the levels of diacetylspermine, diacetylspermidine, N-(3-acetamidopropyl)pyrrolidin-2-one, N-acetylneuraminate, N-acetyl-mannosamine, N-acetyl-lactosamine, and hydroxyisobutyric acid, and optionally, the levels of Human epididymal protein 4 (HE4) and Mucin 16 (CA125), in the biological sample; and
b) classifying the subject as being at risk of harboring ovarian cancer or not being at risk of harboring ovarian cancer based on said measured levels.

Also provided herein is a method of calculating a patient's biomarker score or risk score of harboring ovarian cancer, comprising, in a biological sample obtained from the subject:
a) measuring the levels of diacetylspermine, diacetylspermidine, N-(3-acetamidopropyl)pyrrolidin-2-one, N-acetylneuraminate, N-acetyl-mannosamine, N-acetyl-lactosamine, and hydroxyisobutyric acid, and optionally, the levels of Human epididymal protein 4 (HE4) and Mucin 16 (CA125), in the biological sample; and
b) calculating the biomarker score or risk score using the numerical values of the measured levels in a deep learning model (DLM).

Also provided herein is a method of method of risk stratification for a patient at risk of harboring ovarian cancer, comprising, in a biological sample obtained from the subject:
a) measuring the levels of diacetylspermine, diacetylspermidine, N-(3-acetamidopropyl)pyrrolidin-2-one, N-acetylneuraminate, N-acetyl-mannosamine, N-acetyl-lactosamine, and hydroxyisobutyric acid, and optionally, the levels of Human epididymal protein 4 (HE4) and Mucin 16 (CA125), in the biological sample; and
b) determining, by processor circuitry, the risk score for the patient, wherein the risk score is determined via a scoring function derived from metabolite profiles for biological samples taken from a plurality of individuals that were monitored for ovarian cancer.

### BRIEF DESCRIPTION OF THE DRAWINGS

FIG. 1 depicts the predictive performance of the 7MetP for distinguishing early-stage ovarian cancer from benign pelvic masses in the independent Test Set.

FIG. 2 depicts the schematic workflow of analyses of the different datasets.

FIG. 3 depicts a Spearman correlation heatmap for the metabolites of the 7MetP in the Training Set.

### DETAILED DESCRIPTION

Provided herein is a method of treatment of ovarian cancer in a patient having elevated levels of diacetylspermine, diacetylspermidine, N-(3-acetamidopropyl)pyrrolidin-2-one, N-acetylneuraminate, N-acetyl-mannosamine, N-acetyl-lactosamine, and hydroxyisobutyric acid, and optionally, elevated levels of Human epididymal protein 4 (HE4) and Mucin 16 (CA125), wherein the elevated levels classify the patient as having ovarian cancer, comprising administering a therapeutically effective amount of a treatment for ovarian cancer to the patient.

Also provided herein is a method of treatment of ovarian cancer, comprising:
a) identifying a patient having elevated levels of diacetylspermine, diacetylspermidine, N-(3-acetamidopropyl)pyrrolidin-2-one, N-acetylneuraminate, N-acetyl-mannosamine, N-acetyl-lactosamine, and hydroxyisobutyric acid, and optionally, elevated levels of Human epididymal protein 4 (HE4) and Mucin 16 (CA125), wherein the elevated levels classify the patient as having ovarian cancer; and
b) administering a therapeutically effective amount of a treatment for ovarian cancer to the patient.

Also provided herein is a method of distinguishing ovarian cancer from a benign pelvic mass (BPM) in a subject, comprising, in a biological sample obtained from the subject:
a) measuring the levels of diacetylspermine, diacetylspermidine, N-(3-acetamidopropyl)pyrrolidin-2-one, N-acetylneuraminate, N-acetyl-mannosamine, N-acetyl-lactosamine, and hydroxyisobutyric acid, and optionally, the levels of Human epididymal protein 4 (HE4) and Mucin 16 (CA125), in the biological sample; and
b) classifying the subject as having either ovarian cancer or a BPM based on said measured levels.

Also provided herein is a method of determining the risk of a subject for harboring ovarian cancer, comprising, in a biological sample obtained from the subject:
a) measuring the levels of diacetylspermine, diacetylspermidine, N-(3-acetamidopropyl)pyrrolidin-2-one, N-acetylneuraminate, N-acetyl-mannosamine, N-acetyl-lactosamine, and hydroxyisobutyric acid, and optionally, the levels of Human epididymal protein 4 (HE4) and Mucin 16 (CA125), in the biological sample; and
b) classifying the subject as being at risk of harboring ovarian cancer or not being at risk of harboring ovarian cancer based on said measured levels.

Also provided herein is a method of producing a risk profile of a subject for harboring ovarian cancer, comprising, in a biological sample obtained from the subject:
a) measuring the levels of diacetylspermine, diacetylspermidine, N-(3-acetamidopropyl)pyrrolidin-2-one, N-acetylneuraminate, N-acetyl-mannosamine, N-acetyl-lactosamine, and hydroxyisobutyric acid, and optionally, the levels of Human epididymal protein 4 (HE4) and Mucin 16 (CA125), in the biological sample; and
b) classifying the subject as being at risk of harboring ovarian cancer or not being at risk of harboring ovarian cancer based on said measured levels.

Also provided herein is a method of calculating a patient's biomarker score or risk score of harboring ovarian cancer, comprising, in a biological sample obtained from the subject:
a) measuring the levels of diacetylspermine, diacetylspermidine, N-(3-acetamidopropyl)pyrrolidin-2-one, N-acetylneuraminate, N-acetyl-mannosamine, N-acetyl-lactosamine, and hydroxyisobutyric acid, and optionally, the levels of Human epididymal protein 4 (HE4) and Mucin 16 (CA125), in the biological sample; and
b) calculating the biomarker score or risk score using the numerical values of the measured levels in a deep learning model (DLM).

Also provided herein is a method of method of risk stratification for a patient at risk of harboring ovarian cancer, comprising, in a biological sample obtained from the subject:
a) measuring the levels of diacetylspermine, diacetylspermidine, N-(3-acetamidopropyl)pyrrolidin-2-one, N-acetylneuraminate, N-acetyl-mannosamine, N-acetyl-lactosamine, and hydroxyisobutyric acid, and optionally, the levels of Human epididymal protein 4 (HE4) and Mucin 16 (CA125), in the biological sample; and
b) determining, by processor circuitry, the risk score for the patient, wherein the risk score is determined via a scoring function derived from metabolite profiles for biological samples taken from a plurality of individuals that were monitored for ovarian cancer.

In some embodiments, the DLM comprises an artificial neural network which has between one and three hidden layers and between one and three nodes in each layer.

In some embodiments, the DLM comprises an artificial neural network which has three hidden layers and three nodes in each layer.

In some embodiments, the method further comprises measuring the levels of or identifying a patient with elevated levels of Human epididymal protein 4 (HE4) and Mucin 16 (CA125).

In some embodiments, the levels of HE4 and CA125 are determined by an immunoassay.

In some embodiments, the levels of HE4 and CA125 are used to calculate a predictive index (PI) for premenopausal women with the equation:

PI=−12.0+2.38*ln[HE4]+0.0626*ln[CA125].

In some embodiments, the levels of HE4 and CA125 are used to calculate a predictive index (PI) for postmenopausal women with the equation:

$$PI = - 8.09 + 1.04 * ln [ HE 4 ] + 0.732 * ln [ CA 125 ] .$$

In some embodiments, the predictive index (PI) is used to calculate a Risk of Ovarian Malignancy (ROMA) score with the equation:

$$ROMA score ( \% ) = \frac{exp ( PI )}{1 + exp ( PI )} * 100$$

In some embodiments, a combined model score is calculated using a logistic regression with the ROMA score and the biomarker score.

In some embodiments, the ovarian cancer is early stage (e.g., stage I or II).

In some embodiments, the ovarian cancer is advanced (e.g., stage III or IV).

In some embodiments, the levels of diacetylspermine, diacetylspermidine, N-(3-acetamidopropyl)pyrrolidin-2-one, N-acetylneuraminate, N-acetyl-mannosamine, N-acetyl-lactosamine, and hydroxyisobutyric acid, and optionally, HE4 and CA125, are elevated relative to a reference patient or group that does not have ovarian cancer.

In some embodiments, the levels of diacetylspermine, diacetylspermidine, N-(3-acetamidopropyl)pyrrolidin-2-one, N-acetylneuraminate, N-acetyl-mannosamine, N-acetyl-lactosamine, and hydroxyisobutyric acid, and optionally, HE4 and CA125, are elevated relative to a reference patient or group that has a benign pelvic mass (BPM).

In some embodiments, the subject presents with a pelvic mass.

In some embodiments, each of diacetylspermine, diacetylspermidine, N-(3-acetamidopropyl)pyrrolidin-2-one, N-acetylneuraminate, N-acetyl-mannosamine, N-acetyl-lactosamine, and hydroxyisobutyric acid, and optionally, HE4 and CA125, generates a detectable signal.

In some embodiments, the detectable signals are detectable by a spectrometric method.

In some embodiments, the spectrometric method is chosen from UV-visible spectroscopy, mass spectroscopy, nuclear magnetic resonance (NMR) spectroscopy, proton NMR spectroscopy, nuclear magnetic resonance (NMR) spectrometry, gas chromatography, mass spectrometry (GC-MS), liquid chromatography-mass spectrometry (LC-MS), correlation spectroscopy (COSY), nuclear Overhauser effect spectroscopy (NOESY), rotating-frame nuclear Overhauser effect spectroscopy (ROESY), time-of-flight LC-MS (LC-TOF-MS), liquid chromatography-tandem mass spectrometry (LC-MS/MS), and capillary electrophoresis-mass spectrometry.

In some embodiments, the spectrometric method is mass spectrometry.

In some embodiments, the mass spectrometry is LC-TOF-MS.

In some embodiments, the treatment is chosen from surgery, chemotherapy, immunotherapy, radiation therapy, targeted therapy, or a combination thereof.

In some embodiments, the measured levels are used to calculate a biomarker score or risk profile based on sensitivity and specificity values that corresponds to the risk of the subject for harboring ovarian cancer.

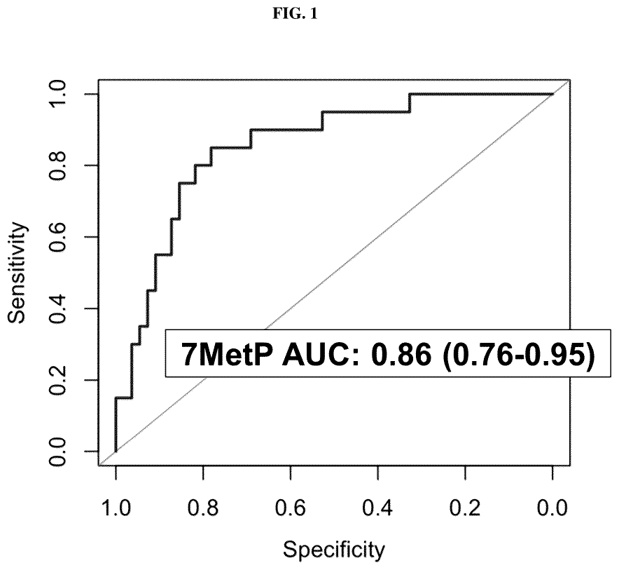

In some embodiments, the sensitivity and specificity values do not differ substantially from the curve in FIG. 1.

In some embodiments, the sensitivity and specificity values differ by less than 10%.

In some embodiments, the sensitivity and specificity values differ by less than 5%.

In some embodiments, the sensitivity and specificity values differ by less than 1%.

In some embodiments, the cutoff value comprises an AUC (95% CI) of at least 0.76.

In some embodiments, the method further comprises assigning the patient to an appropriate risk group based on the calculated risk score.

In some embodiments, there are at least two risk groups.

In some embodiments, the AUC of the method is greater than the AUC for a different biomarker, biomarkers, panel, assay, or algorithm incorporating a combination thereof.

In some embodiments, the AUC is greater than 0.76.

In some embodiments, the AUC is between 0.76 and 0.95.

In some embodiments, the AUC is about 0.88.

In some embodiments, the AUC is about 0.86.

In some embodiments, the AUC is between 0.82 and 0.93

In some embodiments, the AUC is about 0.87.

In some embodiments, the positive predictive value (PPV) of the method is greater than the AUC for a different biomarker, biomarkers, panel, assay, or algorithm incorporating a combination thereof.

In some embodiments, the PPV is greater than 0.67.

In some embodiments, the PPV is between 0.67 and 0.87.

In some embodiments, the PPV is about 0.79.

In some embodiments, the algorithm is the Risk of Ovarian Malignancy Algorithm (ROMA).

In some embodiments, the biomarkers are HE4 and CA125 alone.

In some embodiments, the cutoff points of the respective methods are used for classification.

In some embodiments, the different biomarkers, panels, assays, or algorithms are analyzed by the same statistical methods.

In some embodiments, the levels of diacetylspermine, diacetylspermidine, N-(3-acetamidopropyl)pyrrolidin-2-one, N-acetylneuraminate, N-acetyl-mannosamine, N-acetyl-lactosamine, and hydroxyisobutyric acid are measured against a given threshold value or values.

In some embodiments, the values exceed the threshold value or values and the patient is classified as being at risk of harboring ovarian cancer.

In some embodiments, the values are below the threshold value or values and the patient is classified as being not at risk of harboring ovarian cancer.

In some embodiments, the values are below the threshold value or values and the patient is classified as having a BPM.

In some embodiments, the patient is subsequently designated for further ovarian cancer screening or treatment.

In some embodiments, the screening is chosen from endopic ultrasound, magnetic resonance imaging (MRI), and computed topography (CT) scans.

In some embodiments, the screening is performed annually.

In some embodiments, the screening is performed semi-annually.

In some embodiments, the methods disclosed above and herein may optionally also include combinations of metabolites chosed from those metabolites disclosed in Table 4 below having a p-value of <0.05 (at minimum) when considering either all cases vs. controls OR early stage cases vs. controls.

### Definitions

As used herein, the terms below have the meanings indicated.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a range. When no particular range, such as a margin of error or a standard deviation to a mean value given in a chart or table of data, is recited, the term “about” should be understood to mean the greater of the range which would encompass the recited value and the range which would be included by rounding up or down to that figure as well, taking into account significant figures, and the range which would encompass the recited value plus or minus 20%.

As used herein, “ovarian cancer” refers to a malignant growth of cells that forms in the ovary. Ovarian cancer is most commonly epithelial in origin (around 90% of ovarian cancers), and can be classified into a variety of types, including serous and non-serous ovarian cancer. Serous ovarian cancer is the most common type of epithelial cell ovarian cancer and accounts for around 40% of all ovarian cancers, while non-serous ovarian cancer may include, but is not limited to, endometrioid, mucinous, and clear cell carcinoma. In some embodiments, ovarian cancer may vary in severity, represented by stages I through IV. In some embodiments, ovarian cancer may be in an early stage (e.g., stage I or II), or it may be advanced (e.g., stage III or IV).

When a group is defined to be “null,” what is meant is that said group is absent.

As used herein, the terms “subject” or “patient” refer to a mammal, preferably a human, for whom a classification as ovarian cancer-positive or ovarian cancer-negative is desired, and for whom further treatment can be provided.

As used herein, a “reference patient,” “reference subject,” or “reference group” refers to a group of patients or subjects to which a test sample from a patient or subject suspected of having or being at risk for ovarian cancer may be compared. In some embodiments, such a comparison may be used to determine whether the test subject has ovarian cancer. A reference patient or group may serve as a control for testing or diagnostic purposes. As described herein, a reference patient or group may be a sample obtained from a single patient, or may represent a group of samples, such as a pooled group of samples.

As used herein, “healthy” refers to an individual in whom no evidence of ovarian cancer is found, i.e., the individual does not have ovarian cancer. Such an individual may be classified as “ovarian cancer-negative” or as having healthy ovaries, or normal, non-compromised ovarian function. A healthy patient or subject has no symptoms of ovarian cancer or other ovarian disease, but may have benign pelvic masses, i.e., a combination of adenomas and cysts. In some embodiments, a healthy patient or subject may be used as a reference patient for comparison to diseased or suspected diseased samples for determination of ovarian cancer in a patient or a group of patients.

As used herein, the terms “treatment” or “treating” refer to the administration of medicine or the performance of medical procedures with respect to a subject, for either prophylaxis (prevention) or to cure or reduce the extent of or likelihood of occurrence or recurrence of the infirmity or malady or condition or event in the instance where the subject or patient is afflicted. As related to the present disclosure, the term may also mean the administration of pharmacological substances or formulations, or the performance of non-pharmacological methods including, but not limited to, radiation therapy and surgery. Pharmacological substances as used herein may include, but are not limited to, anticancer drugs including chemotherapeutics, polyamine inhibitors, hormone therapies, and targeted therapies. Examples of chemotherapeutics for ovarian cancer include paclitaxel (e.g. albumin bound paclitaxel or nab-paclitaxel, trade name Abraxane®), altretamine (Hexalen®), capecitabine (Xeloda®), cyclophosphamide (Cytoxan®), etoposide (VP-16), gemcitabine (Gemzar®), ifosfamide (Ifex®), irinotecan (CPT-11, Camptosar®), liposomal irinotecan (Onivyde®), liposomal doxorubicin (Doxil®), melphalan, pemetrexed (Alimta®), topotecan, and vinorelbine (Navelbine®); as well as combination regimens of chemotherapy including cisplatin+paclitaxel, TIP (paclitaxel/Taxol, ifosfamide, and cisplatin/Platinol), VeIP (vinblastine, ifosfamide, and cisplatin/Platinol), VIP (etoposide/VP-16, ifosfamide, and cisplatin/Platinol), VAC (vincristine, dactinomycin, and cyclophosphamide), and PEB (cisplatin/Platinol, etoposide, and bleomycin). Examples of polyamine inhibitors that have been or are being explored in clinical trials for anti-cancer treatment include (but are not limited to) eflornithine (Vaniqa®) and AMXT-1501 dicaprate. Examples of hormone therapies for ovarian cancer include luteinizing-hormone-releasing hormone (LHRH) agonists (such as goserelin (Zoladex®) and leuprolide (Lupron®)), tamoxifen, and aromatase inhibitors (such as letrozole (Femara®), anastrozole (Arimidex®), and exemestane (Aromasin®)). Examples of targeted therapies for ovarian cancer include angiogenesis inhibitors such as bevacizumab (Avastin) as well as (poly(ADP)-ribose polymerase) (PARP) inhibitors such as Olaparib (Lynparza), rucaparib (Rubraca), and niraparib (Zejula). The terms “pharmacological substance” and “anticancer therapy” may also include substances used in immunotherapy, such as checkpoint inhibitors. Treatment may include a multiplicity of pharmacological substances, or a multiplicity of treatment methods, including, but not limited to, surgery and chemotherapy.

As used herein, “amount” or “level” refers to a typically quantifiable measurement for a biomarker described herein, wherein the measurement enables comparison of the marker between samples and/or to control samples. In some embodiments, an amount or level is quantifiable and refers to the levels of a particular marker in a biological sample (e.g., blood, serum, urine, etc), as determined by laboratory methods or tests such as an immunoassay, (e.g., antibodies), mass spectrometry, or liquid chromatography. In some embodiments, a marker may be present in the sample in an increased amount, or in a decreased amount. Marker comparisons may be based on direct measurement of the levels of a biomarker described herein, (e.g., through protein quantification or gene expression analysis) or may be based on measurement of e.g., reporter molecules, biomarker-receptor complexes, biomarker-relay-receptor complexes, or the like.

As used herein, the term “ELISA” refers to enzyme-linked immunosorbent assay. This assay generally involves contacting a fluorescently tagged sample of proteins with antibodies having specific affinity for those proteins. Detection of these proteins can be accomplished with a variety of means, including but not limited to laser fluorimetry.

As used herein, the term “regression” refers to a statistical method that can assign a predictive value for an underlying characteristic of a sample based on an observable trait (or set of observable traits) of said sample. In some embodiments, the characteristic is not directly observable. For example, the regression methods used herein can link a qualitative or quantitative outcome of a particular biomarker test, or set of biomarker tests, on a certain subject, to a probability that said subject is for ovarian cancer positive.

As used herein, the term “logistic regression” refers to a regression method in which the assignment of a prediction from the model can have one of several allowed discrete values. For example, the logistic regression models used herein can assign a prediction, for a certain subject, of either ovarian cancer-positive or ovarian cancer-negative.

As used herein, the term “biomarker score” refers to a numerical score for a particular subject that is calculated by inputting the particular biomarker levels for said subject to a statistical method.

As used herein, the term “composite score” refers to a summation of the normalized values for the predetermined markers measured in the sample from the subject. In one embodiment, the normalized values are reported as a biomarker score and those biomarker score values are then summed to provide a composite score for each subjected tested. When used in the context of the risk categorization table and correlated to a stratified grouping based on a range of composite scores in the Risk Categorization Table, the “composite score” is used to determine the “risk score” for each subject tested wherein the multiplier indicating increased likelihood of having the cancer for the stratified grouping becomes the “risk score”.

As used herein, the term “risk score” refers to a single numerical value that indicates an asymptomatic human subject's risk for ovarian cancer as compared to the known prevalence of ovarian cancer in the disease cohort. In certain embodiments, the composite score as calculated for a human subject and correlated to a multiplier indicating risk for ovarian cancer, wherein the composite score is correlated based on the range of composite scores for each stratified grouping in the risk categorization table. In this way the composite score is converted to a risk score based on the multiplier indicating increased likelihood of having the cancer for the grouping that is the best match for the composite score.

As used herein, the term “cutoff” or “cutoff point” refers to a mathematical value associated with a specific statistical method that can be used to assign a classification of ovarian cancer-positive of ovarian cancer-negative to a subject, based on said subject's biomarker score.

As used herein, when a numerical value above or below a cutoff value “is characteristic of ovarian cancer,” what is meant is that the subject, analysis of whose sample yielded the value, either has ovarian cancer or is at risk for ovarian cancer.

As used herein, the “use” of markers for diagnosing ovarian cancer refers to quantification of the levels or amounts in a biological sample of one or more markers described herein. Quantification may be done using any known methods or techniques in the art or described herein. In some embodiments, markers may be used or combined together as a panel for statistical comparison to other samples.

In some embodiments, the amount or levels of DAS, NANA, NAcMan, NAcLac, DiAcSpmd, N3AP, and HBA, or the amount or levels of DAS, NANA, NAcMan, NAcLac, DiAcSpmd, N3AP, HBA, HE4, and CA125, are compared with a cutoff value comprising an AUC (95% CI) of from about 0.48 to about 0.88, e.g., about 0.48, about 0.49, about 0.50, about 0.51, about 0.52, about 0.52, about 0.53, about 0.54, about 0.55, about 0.56, about 0.57, about 0.58, about 0.59, about 0.60, about 0.61, about 0.62, about 0.63, about 0.64, about 0.65, about 0.66, about 0.67, about 0.68, about 0.69, about 0.70, about 0.71, about 0.72, about 0.73, about 0.74, about 0.75, about 0.76, about 0.77, about 0.78, about 0.79, about 0.80, about 0.81, about 0.82, about 0.83, about 0.84, about 0.85, about 0.86, about 0.87, about 0.88, or the like. In some embodiments, using markers DAS, NANA, NAcMan, NAcLac, DiAcSpmd, N3AP, and HBA together as a panel, or using markers DAS, NANA, NAcMan, NAcLac, DiAcSpmd, N3AP, HBA, HE4, and CA125 together as a panel may have an AUC (95% CI) of 0.66 or greater, including about 0.66, about 0.67, about 0.68, about 0.69, about 0.70, about 0.71, about 0.72, about 0.73, about 0.74, about 0.75, about 0.76, about 0.77, about 0.78, about 0.79, about 0.80, about 0.81, about 0.82, about 0.83, about 0.84, about 0.85, about 0.86, about 0.87, about 0.88. about 0.89, about 0.90, about 0.91, about 0.92, about 0.93, about 0.94, about 0.95, about 0.96, about 0.97, about 0.98, about 0.99, or the like.

In some embodiments, analyzing markers DAS, NANA, NAcMan, NAcLac, DiAcSpmd, N3AP, and HBA together as a panel for diagnosis of ovarian cancer using fixed coefficients, or analyzing markers DAS, NANA, NAcMan, NAcLac, DiAcSpmd, N3AP, HBA, HE4, and CA125 together as a panel for diagnosis of ovarian cancer using fixed coefficients may result in an AUC (95% CI) of from about 0.82 to about 0.93 for distinguishing ovarian cancer cases from individuals with benign disease, e.g., about 0.82, about 0.83, about 0.84, about 0.85, about 0.86, about 0.87, about 0.88. about 0.89, about 0.90, about 0.91, about 0.92, about 0.93 or the like. In some embodiments, analyzing these marker panels using fixed coefficients may result in an AUC (95% CI) of 0.88 for distinguishing ovarian cancer cases from individuals with benign disease. In some embodiments, analyzing any of the marker panels described herein for diagnosis of ovarian cancer using fixed coefficients may result in an AUC (95% CI) of from about 0.76 to about 0.95 for distinguishing early-stage ovarian cancer, e.g., about 0.76, about 0.77, about 0.78, about 0.79, about 0.80, about 0.81, about 0.82, about 0.83, about 0.84, about 0.85, about 0.86, about 0.87, about 0.88. about 0.89, about 0.90, about 0.91, about 0.92, about 0.93, about 0.94, about 0.95 or the like. In some embodiments, analyzing these marker panels using fixed coefficients may result in an AUC (95% CI) of 0.86 for distinguishing early-stage ovarian cancer.

In some embodiments, the cutoff value for DAS comprises an AUC (95% CI) of at least 0.76, e.g., about 0.76, about 0.77, about 0.78, about 0.79, about 0.80, about 0.81, about 0.82, about 0.83, about 0.84, about 0.85, about 0.86, about 0.87, about 0.88. about 0.89, about 0.90, about 0.91, about 0.92, about 0.93, about 0.94, about 0.95, about 0.96, about 0.97, about 0.98, about 0.99, or the like.

In some embodiments, the cutoff value for NANA comprises an AUC (95% CI) of at least 0.58, e.g., about 0.58, about 0.59, about 0.60, about 0.61, about 0.62, about 0.63, about 0.64, about 0.65, about 0.66, about 0.67, about 0.68, about 0.69, about 0.70, about 0.71, about 0.72, about 0.73, about 0.74, about 0.75, about 0.76, about 0.77, about 0.78, about 0.79, about 0.80, about 0.81, about 0.82, about 0.83, about 0.84, about 0.85, about 0.86, about 0.87, about 0.88. about 0.89, about 0.90, about 0.91, about 0.92, about 0.93, about 0.94, about 0.95, about 0.96, about 0.97, about 0.98, about 0.99, or the like.

In some embodiments, the cutoff value for NAcMan comprises an AUC (95% CI) of at least 0.50, e.g., about 0.50, about 0.51, about 0.52, about 0.53, about 0.54, about 0.55, about 0.56, about 0.57, about 0.58, about 0.59, about 0.60, about 0.61, about 0.62, about 0.63, about 0.64, about 0.65, about 0.66, about 0.67, about 0.68, about 0.69, about 0.70, about 0.71, about 0.72, about 0.73, about 0.74, about 0.75, about 0.76, about 0.77, about 0.78, about 0.79, about 0.80, about 0.81, about 0.82, about 0.83, about 0.84, about 0.85, about 0.86, about 0.87, about 0.88. about 0.89, about 0.90, about 0.91, about 0.92, about 0.93, about 0.94, about 0.95, about 0.96, about 0.97, about 0.98, about 0.99, or the like.

In some embodiments, the cutoff value for NAcLac comprises an AUC (95% CI) of at least 0.48, e.g., about 0.48, about 0.49, about 0.50, about 0.51, about 0.52, about 0.53, about 0.54, about 0.55, about 0.56, about 0.57, about 0.58, about 0.59, about 0.60, about 0.61, about 0.62, about 0.63, about 0.64, about 0.65, about 0.66, about 0.67, about 0.68, about 0.69, about 0.70, about 0.71, about 0.72, about 0.73, about 0.74, about 0.75, about 0.76, about 0.77, about 0.78, about 0.79, about 0.80, about 0.81, about 0.82, about 0.83, about 0.84, about 0.85, about 0.86, about 0.87, about 0.88. about 0.89, about 0.90, about 0.91, about 0.92, about 0.93, about 0.94, about 0.95, about 0.96, about 0.97, about 0.98, about 0.99, or the like.

In some embodiments, the cutoff value for DiAcSpmd comprises an AUC (95% CI) of at least 0.60, e.g., about 0.60, about 0.61, about 0.62, about 0.63, about 0.64, about 0.65, about 0.66, about 0.67, about 0.68, about 0.69, about 0.70, about 0.71, about 0.72, about 0.73, about 0.74, about 0.75, about 0.76, about 0.77, about 0.78, about 0.79, about 0.80, about 0.81, about 0.82, about 0.83, about 0.84, about 0.85, about 0.86, about 0.87, about 0.88. about 0.89, about 0.90, about 0.91, about 0.92, about 0.93, about 0.94, about 0.95, about 0.96, about 0.97, about 0.98, about 0.99, or the like.

In some embodiments, the cutoff value for N3AP comprises an AUC (95% CI) of at least 0.49, e.g., about 0.49, about 0.50, about 0.51, about 0.52, about 0.53, about 0.54, about 0.55, about 0.56, about 0.57, about 0.58, about 0.59, about 0.60, about 0.61, about 0.62, about 0.63, about 0.64, about 0.65, about 0.66, about 0.67, about 0.68, about 0.69, about 0.70, about 0.71, about 0.72, about 0.73, about 0.74, about 0.75, about 0.76, about 0.77, about 0.78, about 0.79, about 0.80, about 0.81, about 0.82, about 0.83, about 0.84, about 0.85, about 0.86, about 0.87, about 0.88. about 0.89, about 0.90, about 0.91, about 0.92, about 0.93, about 0.94, about 0.95, about 0.96, about 0.97, about 0.98, about 0.99, or the like.

In some embodiments, the cutoff value for HBA comprises an AUC (95% CI) of at least 0.64, e.g., about 0.64, about 0.65, about 0.66, about 0.67, about 0.68, about 0.69, about 0.70, about 0.71, about 0.72, about 0.73, about 0.74, about 0.75, about 0.76, about 0.77, about 0.78, about 0.79, about 0.80, about 0.81, about 0.82, about 0.83, about 0.84, about 0.85, about 0.86, about 0.87, about 0.88. about 0.89, about 0.90, about 0.91, about 0.92, about 0.93, about 0.94, about 0.95, about 0.96, about 0.97, about 0.98, about 0.99, or the like.

As used herein, a subject who is at “risk for ovarian cancer” is one who may not yet evidence overt symptoms of ovarian cancer, but who is producing levels of biomarkers which indicate that the subject has ovarian cancer, or may develop it in the near term. A subject who has ovarian cancer or is suspected of harboring ovarian cancer may be treated for the cancer or suspected cancer.

As used herein, the term “classification” refers to the assignment of a subject as being at risk for ovarian cancer or not being at risk for ovarian cancer, based on the result of the biomarker score that is obtained for said subject.

As used herein, the term “Wilcoxon rank sum test,” also known as the Mann-Whitney U test, Mann-Whitney-Wilcoxon test, or Wilcoxon-Mann-Whitney test, refers to a specific statistical method used for comparison of two populations. For example, the test can be used herein to link an observable trait, in particular a biomarker level, to the absence or the risk for ovarian cancer in subjects of a certain population.

As used herein, the term “positive predictive value” refers to the proportion of positive results derived by a certain method that are truly positive.

As used herein, the term “negative predictive value” refers to the proportion of negative results derived by a certain method that are truly negative.

As used herein, the term “sensitivity” refers to, in the context of various biochemical assays, the ability of an assay to correctly identify those with a disease (i.e., the true positive rate). By comparison, as used herein, the term “specificity” refers to, in the context of various biochemical assays, the ability of an assay to correctly identify those without the disease (i.e., the true negative rate). Sensitivity and specificity are statistical measures of the performance of a binary classification test (i.e., classification function). Sensitivity quantifies the avoiding of false negatives, and specificity does the same for false positives.

As used herein, “fixed coefficients” or “fixed model coefficients” refers to a statistical method of standardizing coefficients in order to allow comparison of the relative importance of each coefficient in a regression model. In some embodiments, fixed coefficients involves using the same beta-coefficients from a logistic regression model to yield a composite score for the developed combination rule, which is ultimately used to make a clinical decision based on a decision threshold(s).

As used herein, a “sample” refers to a test substance to be tested for the presence of, and levels or concentrations thereof, of a biomarker as described herein. A sample may be any substance appropriate in accordance with the present disclosure, including, but not limited to, blood, blood serum, blood plasma, or any part thereof.

As used herein, a “metabolite” refers to small molecules that are intermediates and/or products of cellular metabolism. Metabolites may perform a variety of functions in a cell, for example, structural, signaling, stimulatory and/or inhibitory effects on enzymes. In some embodiments, a metabolite may be a non-protein, plasma-derived metabolite marker, such as including, but not limited to, DAS, NANA, NAcMan, NAcLac, DiAcSpmd, N3AP, and HBA. In some embodiments, a metabolite useful as described herein may be a “polyamine,” i.e., an organic compound having more than two amino groups. In some embodiments, a polyamine as described herein be a plasma polyamine. In some embodiments, polyamines useful for the present panels and methods include, but are not limited to, DAS, NANA, NAcMan, NAcLac, DiAcSpmd, N3AP, and HBA. These polyamines may be combined with other markers, e.g., CA125 or HE4, for enhanced detection of ovarian cancer as described herein.

As used herein, the term “7-marker metabolite panel” or “7MetP” refers to a panel of seven biomarkers, which includes DAS, NANA, NAcMan, NAcLac, DiAcSpmd, N3AP, and HBA, useful for detecting ovarian cancer in a patient suspected of having ovarian cancer. In some embodiments, the 7-marker metabolite panel may be evaluated in combination with additional markers, such as plasma polyamines, to enhance detection of ovarian cancer in biological samples from patients suspected as having ovarian cancer. Useful plasma polyamines include, but are not limited to, N3AP, AcSpmd, DiAcSpmd, and/or DAS. Additional polyamines are known in the art and may be included as deemed appropriate by a clinician.

As used herein, the term “ROC” refers to receiver operating characteristic, which is a graphical plot used herein to gauge the performance of a certain diagnostic method at various cutoff points. A ROC plot can be constructed from the fraction of true positives and false positives at various cutoff points.

As used herein, the term “AUC” refers to the area under the curve of the ROC plot. AUC can be used to estimate the predictive power of a certain diagnostic test. Generally, a larger AUC corresponds to increasing predictive power, with decreasing frequency of prediction errors. Possible values of AUC range from 0.5 to 1.0, with the latter value being characteristic of an error-free prediction method.

As used herein, the term “p-value” or “p” refers to the probability that the distributions of biomarker scores for ovarian cancer-positive and ovarian cancer-negative subjects are identical in the context of a Wilcoxon rank sum test. Generally, a p-value close to zero indicates that a particular statistical method will have high predictive power in classifying a subject.

As used herein, the term “CI” refers to a confidence interval, i.e., an interval in which a certain value can be predicted to lie with a certain level of confidence. As used herein, the term “95% CI” refers to an interval in which a certain value can be predicted to lie with a 95% level of confidence.

As used herein, the term “disease progression” or “early disease progression” is defined as upgrading of Gleason score and/or increased tumor volume on surveillance biopsy within 18 months after start of active surveillance.

The phrase “therapeutically effective” is intended to qualify the amount of active ingredients used in the treatment of a disease or disorder or on the effecting of a clinical endpoint.

### LIST OF ABBREVIATIONS

AUC=area under the curve; DAS=N1, N12-diacetylspermine; DiAcSpmd=N1,N8-diacetyl spermidine; HBA=hydroxyisobutyric acid; HILIC=hydrophilic interaction liquid chromatography; HPLC=high performance liquid chromatography; N3AP=N-(3-acetamidopropyl)pyrrolidin-2-one; NANA=N-acetylneuraminate; NAcMan=N-acetyl-mannosamine; NAcLac=N-acetyl-lactosamine; OvCa=ovarian cancer; ROC=receiver operating characteristic; SEM=standard error of the mean; TCGA=The Cancer Genome Atlas; UPLC=ultra high performance liquid chromatography; UPLC/MS=ultra high performance liquid chromatography/mass spectrometry.

### EXAMPLES

The following examples are included to demonstrate embodiments of the disclosure. The following examples are presented only by way of illustration and to assist one of ordinary skill in using the disclosure. The examples are not intended in any way to otherwise limit the scope of the disclosure. Those of skill in the art should, in light of the present disclosure, appreciate that many changes can be made in the specific embodiments which are disclosed and still obtain a like or similar result without departing from the spirit and scope of the disclosure.

### Example 1: Specimen Sets

Blood specimens were obtained preoperatively with informed consent under IRB/ethical committees approved protocols (LAB04-0687) at the University of Texas M.D. Anderson Cancer Center (MDACC) and at the Fred Hutchinson Cancer Research Center (FHCRC, IRB 4563) from patients who were admitted for surgery based on a mass found on ultrasound, elevated CA125, or a positive biopsy. All patients were fasting at the time of blood collection. Samples were processed on the same day, generally within 4 hours of blood draw, under standardized operating procedures, aliquoted to minimize freeze-thaw cycling effects, and stored in −80° C. until use. The specimen set consisted of plasma from 59 patients with stage I-II and 160 patients with stage III-IV invasive epithelial ovarian cancer and from 190 patients with benign pelvic masses. Biopsy samples were examined by a certified pathologist for the diagnosis of cancer or benign pelvic condition. Detailed patient and tumor characteristics are provided in Table 1. Information regarding histological ovarian cancer subtypes and benign etiologies are provided in Table 2. All participants had provided consent for use of samples in ethically approved secondary studies.

TABLE 1
Patient and Tumor characteristics

| Patient and Tumor | Training Set | | | | | Testing Set | | | | |
|---|---|---|---|---|---|---|---|---|---|---|
| Characteristics | Cases | | BPM† | | P-value‡ | Cases | | BPM† | | P-value‡ |
| Number of Subjects, N | 101 | | 134 | | | 118 | | 56 | | |
| Age, average (min-max) | 59 | (23-85) | 57 | (30-83) | 0.14 | 61 | (36-86) | 55 | (33-79) | 0.009 |
| Menopausal Status, N (%) | | | | | | | | | | |
| Pre | 21 | (20.8%) | 39 | (29.1%) | 0.17 | 19 | (16.1%) | 18 | (32.1%) | 0.02 |
| Post | 80 | (79.2%) | 95 | (70.9%) | | 99 | (83.8%) | 38 | (67.8%) | |
| Race, N (%) | | | | | | | | | | |
| White | 83 | (82.2%) | 104 | (77.6%) | 0.18 | 87 | (73.7%) | 42 | (75%) | 0.52 |
| Black | 5 | (5%) | 3 | (2.2%) | | 9 | (7.6%) | 9 | (16.1%) | |
| Asian | 2 | (2%) | 3 | (2.2%) | | 13 | (11%) | 1 | (1.8%) | |
| American Indian | — | | 1 | (0.7%) | | — | | — | | |
| Pacific Islander | — | | — | | | 9 | (7.6%) | 4 | (7.1%) | |
| Unknown | 11 | (10.9%) | 23 | (17.2%) | | — | | — | | |
| Stage, N (%) | | | | | | | | | | |
| Stage I and II | 39 | (38.6%) | — | | | 20 | (16.9%) | — | | |
| Stage III and IV | 62 | (61.4%) | — | | | 98 | (83.1%) | — | | |
| Histological Subtype, N (%) | | | | | | | | | | |
| Serous | 69 | (68.3%) | — | | | 114 | (96.6%) | — | | |
| Non-Serous | 32 | (31.7%) | — | | | 1 | (0.8%) | — | | |
| Unknown | | | | | | 3 | (2.54%) | | | |
| ROMA, median | 86.85 | (36.85-98.03) | 11.14 | (6.64-20.66) | <0.0001 | 89.51 | (70.55-96.95) | 13.66 | (6.42-18.56) | <0.0001 |

(25^{th}/75^{th} Percentiles)

†Individuals with benign pelvic masses (BPM)
‡Statistical significance was determined by Wilcoxon rank sum tests for continuous variables and Fisher's exact test or ² tests for trend for categorical variables. 2-sided p-values are reported.

TABLE 2
Characteristics of Ovarian Cancers and Benign Pelvic Masses

| | | Cancer | Early/Late |
|---|---|---|---|
| Type | Subtype | Stage | Stage |
| Benign | Mucinous Cystadenoma | — | — |
| Benign | Luteal Cyst | — | — |
| Benign | Mucinous Cystadenofribroma | — | — |
| Benign | Cystadenofibroma | — | — |
| Benign | Mucinous Cystadenoma | — | — |
| Benign | Endometriotic Cyst | — | — |
| Benign | Mucinous Cystadenoma | — | — |
| Benign | Endometriosis | — | — |
| Benign | Peritoneal Inclusion Cysts, Follicular Cysts, Corpus Luteal Cysts | — | — |
| Benign | serous cystadenoma | — | — |
| Benign | mucinous cystadenoma | — | — |
| Benign | Sertoli-Leydig cell | — | — |
| Benign | endometrioma | — | — |
| Benign | mucinous cystadenoma | — | — |
| Benign | endometrioic cysts | — | — |
| Benign | bilateral endometriomas | — | — |
| Benign | brenner tumor | — | — |
| Benign | cyst | — | — |
| Benign | mucinous cystadenoma | — | — |
| Benign | endometrioisis, atypia | — | — |
| Benign | endometrioma, endometriosis | — | — |
| Benign | Cyst & Rete Cystadonoma | — | — |
| Benign | Benign Luteal Cyst | — | — |
| Benign | cystadenoma and severe pelvic adhesive disease | — | — |
| Benign | mucinous neoplasm | — | — |
| Benign | serous cystadenomas | — | — |
| Benign | cysts | — | — |
| Benign | Mucinous Cystadenofribroma | — | — |
| Benign | serous cystadenofibroma | — | — |
| Benign | corpus albicanocyst, fibrosis | — | — |
| Benign | mucinous cystadenoma, intesti-1 | — | — |
| Benign | cystadenoma | — | — |
| Benign | cyst | — | — |
| Benign | mucinous cystadenoma | — | — |
| Benign | fibroma, cysts | — | — |
| Benign | mucinous cystadenoma | — | — |
| Benign | serous cystadenofibroma | — | — |
| Benign | cystadenoma | — | — |
| Benign | serous cystadenoma | — | — |
| Benign | serous cystadenoma | — | — |
| Benign | Cysts | — | — |
| Benign | cyst | — | — |
| Benign | Serous adenofibroma | — | — |
| Benign | cystadenofibroma | — | — |
| Benign | ovarian mass | — | — |
| Benign | mucinous cystadenoma | — | — |
| Benign | fibroma | — | — |
| Benign | fibrothecoma of ROV | — | — |
| Benign | benign mass | — | — |
| Benign | serous cystadenofibroma | — | — |
| Benign | serous cyst | — | — |
| Benign | (LOV) fibroma | — | — |
| Benign | Cysts | — | — |
| Benign | serous cystadenofibroma | — | — |
| Benign | Serous Cystadoma | — | — |
| Benign | serous ovarian cystadenofibroma of LOV | — | — |
| Benign | serous | — | — |
| Benign | mucinous | — | — |
| Benign | serous | — | — |
| Benign | mucinous | — | — |
| Benign | serous | — | — |
| Benign | other | — | — |
| Benign | serous | — | — |
| Benign | non-neo. | — | — |
| Benign | non-neo. | — | — |
| Benign | serous | — | — |
| Benign | other | — | — |
| Benign | serous | — | — |
| Benign | other | — | — |
| Benign | serous | — | — |
| Benign | serous | — | — |
| Benign | serous | — | — |
| Benign | serous | — | — |
| Benign | serous | — | — |
| Benign | serous | — | — |
| Benign | non-neo. | — | — |
| Benign | non-neo. | — | — |
| Benign | serous | — | — |
| Benign | serous | — | — |
| Benign | serous | — | — |
| Benign | non-neo. | — | — |
| Benign | non-neo. | — | — |
| Benign | serous | — | — |
| Benign | serous | — | — |
| Benign | serous | — | — |
| Benign | non-neo. | — | — |
| Benign | serous | — | — |
| Benign | non-neo. | — | — |
| Benign | mucinous | — | — |
| Benign | non-neo. | — | — |
| Benign | other | — | — |
| Benign | other | — | — |
| Benign | serous | — | — |
| Benign | serous | — | — |
| Benign | mucinous | — | — |
| Benign | other | — | — |
| Benign | serous | — | — |
| Benign | serous | — | — |
| Benign | non-neo. | — | — |
| Benign | serous | — | — |
| Benign | serous | — | — |
| Benign | serous | — | — |
| Benign | non-neo. | — | — |
| Benign | serous | — | — |
| Benign | serous | — | — |
| Benign | non-neo. | — | — |
| Benign | non-neo. | — | — |
| Benign | non-neo. | — | — |
| Benign | other | — | — |
| Benign | serous | — | — |
| Benign | serous | — | — |
| Benign | non-neo. | — | — |
| Benign | other | — | — |
| Benign | mucinous | — | — |
| Benign | other | — | — |
| Benign | other | — | — |
| Benign | other | — | — |
| Benign | serous | — | — |
| Benign | non-neo. | — | — |
| Benign | other | — | — |
| Benign | serous | — | — |
| Benign | serous | — | — |
| Benign | non-neo. | — | — |
| Benign | serous | — | — |
| Benign | other | — | — |
| Benign | other | — | — |
| Benign | other | — | — |
| Benign | other | — | — |
| Benign | serous | — | — |
| Benign | other | — | — |
| Benign | non-neo. | — | — |
| Benign | serous | — | — |
| Benign | serous | — | — |
| Benign | mucinous | — | — |
| Benign | serous | — | — |
| Benign | serous | — | — |
| Benign | serous | — | — |
| Benign | other | — | — |
| Benign | other | — | — |
| Benign | other | — | — |
| Benign | mucinous | — | — |
| Benign | serous | — | — |
| Benign | serous | — | — |
| Benign | serous | — | — |
| Benign | serous | — | — |
| Benign | non-neo. | — | — |
| Benign | serous | — | — |
| Benign | serous | — | — |
| Benign | serous | — | — |
| Benign | non-neo. | — | — |
| Benign | non-neo. | — | — |
| Benign | serous | — | — |
| Benign | serous | — | — |
| Benign | serous | — | — |
| Benign | other | — | — |
| Benign | other | — | — |
| Benign | serous | — | — |
| Benign | other | — | — |
| Benign | serous | — | — |
| Benign | non-neo. | — | — |
| Benign | serous | — | — |
| Benign | mucinous | — | — |
| Benign | non-neo. | — | — |
| Benign | non-neo. | — | — |
| Benign | other | — | — |
| Benign | other | — | — |
| Benign | non-neo. | — | — |
| Benign | serous | — | — |
| Benign | serous | — | — |
| Benign | serous | — | — |
| Benign | endometrioid | — | — |
| Benign | other | — | — |
| Benign | other | — | — |
| Benign | non-neo. | — | — |
| Benign | serous | — | — |
| Benign | serous | — | — |
| Benign | serous | — | — |
| Benign | serous | — | — |
| Benign | other | — | — |
| Benign | serous | — | — |
| Benign | serous | — | — |
| Benign | other | — | — |
| Benign | serous | — | — |
| Benign | serous | — | — |
| Benign | other | — | — |
| Benign | serous | — | — |
| Benign | other | — | — |
| Benign | non-neo. | — | — |
| Benign | other | — | — |
| Benign | other | — | — |
| Cancer | Serous Carcinoma | IA | Early |
| Cancer | Serous Carcinoma | IA | Early |
| Cancer | Serous Carcinoma | IA | Early |
| Cancer | Clear Cell Carcinoma | IA | Early |
| Cancer | Clear Cell Carcinoma | IA | Early |
| Cancer | Clear Cell Carcinoma | IA | Early |
| Cancer | Endometrioid | IA | Early |
| Cancer | Endometrioid | IA | Early |
| Cancer | Endometrioid | IA | Early |
| Cancer | Endometrioid | IA | Early |
| Cancer | Mucinous | IA | Early |
| Cancer | Mucinous | IA | Early |
| Cancer | Mucinous | IA | Early |
| Cancer | other | IA | Early |
| Cancer | other | IA | Early |
| Cancer | other | IA | Early |
| Cancer | Serous Carcinoma | IA | Early |
| Cancer | Serous Carcinoma | IA | Early |
| Cancer | Serous Carcinoma | IA | Early |
| Cancer | Serous Carcinoma | IA | Early |
| Cancer | Serous & Endometrioid Carcinoma | IB | Early |
| Cancer | Serous Carcinoma | IC | Early |
| Cancer | Serous Carcinoma | IC | Early |
| Cancer | Serous Carcinoma | IC | Early |
| Cancer | Serous Carcinoma | IC | Early |
| Cancer | Serous Carcinoma | IC | Early |
| Cancer | Clear Cell Carcinoma | IC | Early |
| Cancer | Clear Cell Carcinoma | IC | Early |
| Cancer | Endometrioid | IC | Early |
| Cancer | other | IC | Early |
| Cancer | Serous Carcinoma | IC | Early |
| Cancer | Serous Carcinoma | IC | Early |
| Cancer | Serous Carcinoma | IC | Early |
| Cancer | Serous Carcinoma | IIA | Early |
| Cancer | Serous Carcinoma | IIA | Early |
| Cancer | Serous Carcinoma | IIA | Early |
| Cancer | Clear Cell Carcinoma | IIA | Early |
| Cancer | Clear Cell Carcinoma | IIA | Early |
| Cancer | Serous Carcinoma | IIB | Early |
| Cancer | Serous Carcinoma | IIB | Early |
| Cancer | Serous Carcinoma | IIB | Early |
| Cancer | Serous Carcinoma | IIB | Early |
| Cancer | Serous Carcinoma | IIB | Early |
| Cancer | Adenocarcinoma | IIB | Early |
| Cancer | Serous Carcinoma | IIB | Early |
| Cancer | Endometrioid | IIB | Early |
| Cancer | other | IIB | Early |
| Cancer | other | IIB | Early |
| Cancer | Serous Carcinoma | IIB | Early |
| Cancer | Serous Carcinoma | IIB | Early |
| Cancer | Serous Carcinoma | IIB | Early |
| Cancer | Serous Carcinoma | IIB | Early |
| Cancer | Serous Carcinoma | IIC | Early |
| Cancer | Endometrioid | IIC | Early |
| Cancer | Endometrioid | IIC | Early |
| Cancer | other | IIC | Early |
| Cancer | other | IIC | Early |
| Cancer | Serous Carcinoma | IIC | Early |
| Cancer | Serous Carcinoma | IIC | Early |
| Cancer | Serous Carcinoma | IIC | Late |
| Cancer | Serous Carcinoma | III | Late |
| Cancer | Serous Carcinoma | III | Late |
| Cancer | — | III | Late |
| Cancer | Serous Carcinoma | III | Late |
| Cancer | Serous Carcinoma | III | Late |
| Cancer | Serous Carcinoma | III | Late |
| Cancer | Serous Carcinoma | III | Late |
| Cancer | Serous Carcinoma | III | Late |
| Cancer | Serous Carcinoma | III | Late |
| Cancer | Serous Carcinoma | III | Late |
| Cancer | Serous Carcinoma | III | Late |
| Cancer | Serous Carcinoma | IIIA | Late |
| Cancer | Serous Carcinoma | IIIA | Late |
| Cancer | Serous Carcinoma | IIIA | Late |
| Cancer | Serous Carcinoma | IIIA | Late |
| Cancer | Serous Carcinoma | IIIA | Late |
| Cancer | Serous Carcinoma | IIIA | Late |
| Cancer | Serous Carcinoma | IIIB | Late |
| Cancer | Serous Carcinoma | IIIB | Late |
| Cancer | Endometrioid | IIIB | Late |
| Cancer | other | IIIB | Late |
| Cancer | Serous Carcinoma | IIIB | Late |
| Cancer | Serous Carcinoma | IIIB | Late |
| Cancer | Serous Carcinoma | IIIB | Late |
| Cancer | Serous Carcinoma | IIIB | Late |
| Cancer | Serous Carcinoma | IIIB | Late |
| Cancer | Serous Carcinoma | IIIB | Late |
| Cancer | Serous Carcinoma | IIIB | Late |
| Cancer | Serous Carcinoma | IIIB | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma/Clear Cell Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | — | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma/minor endometrioird adenocarcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Clear Cell Carcinoma | IIIC | Late |
| Cancer | Endometrioid | IIIC | Late |
| Cancer | other | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IIIC | Late |
| Cancer | Serous Carcinoma | IV | Late |
| Cancer | Serous Carcinoma | IV | Late |
| Cancer | Serous carcinoma/undifferentiated carcinoma | IV | Late |
| Cancer | Serous Carcinoma | IV | Late |
| Cancer | Serous Carcinoma | IV | Late |
| Cancer | Serous Carcinoma | IV | Late |
| Cancer | Serous Carcinoma | IV | Late |
| Cancer | Serous Carcinoma | IV | Late |
| Cancer | Serous Carcinoma | IV | Late |
| Cancer | Serous Carcinoma | IV | Late |
| Cancer | Serous Carcinoma | IV | Late |
| Cancer | Serous Carcinoma | IV | Late |
| Cancer | Serous Carcinoma | IV | Late |
| Cancer | Serous Carcinoma | IV | Late |
| Cancer | — | IV | Late |
| Cancer | other | IV | Late |
| Cancer | Serous Carcinoma | IV | Late |
| Cancer | Serous Carcinoma | IV | Late |
| Cancer | Serous Carcinoma | IV | Late |
| Cancer | Serous Carcinoma | IV | Late |
| Cancer | Serous Carcinoma | IV | Late |
| Cancer | Serous Carcinoma | IV | Late |
| Cancer | Serous Carcinoma | IV | Late |
| Cancer | Serous Carcinoma | IV | Late |

### Example 2: Metabolomic Analysis

### Primary Metabolites and Biogenic Amines

Serum metabolites were extracted from pre-aliquoted EDTA plasma (10 μL) with 30 μL of LCMS grade methanol (ThermoFisher) in a 96-well microplate (Eppendorf). Plates were heat sealed, vortexed for 5 min at 750 rpm, and centrifuged at 2000×g for 10 minutes at room temperature. The supernatant (10 μL) was carefully transferred to a 96-well plate, leaving behind the precipitated protein. The supernatant was further diluted with 10 μL of 100 mM ammonium formate, pH 3. For Hydrophilic Interaction Liquid Chromatography (HILIC) analysis, the samples were diluted with 60 μL LCMS grade acetonitrile (ThermoFisher), whereas samples for C18 analysis were diluted with 60 μL water (GenPure ultrapure water system, Thermofisher). Each sample solution was transferred to 384-well microplate (Eppendorf) for LCMS analysis.

### Untargeted Analysis of Primary Metabolites and Biogenic Amines

Untargeted metabolomics analysis was conducted on a Waters Acquity™ UPLC system with 2D column regeneration configuration (I-class and H-class) coupled to a Xevo G2-XS quadrupole time-of-flight (qTOF) mass spectrometer. Chromatographic separation was performed using HILIC (Acquity™ UPLC BEH amide, 100 Å, 1.7 μm 2.1×100 mm, Waters Corporation, Milford, U.S.A) and C18 (Acquity™ UPLC HSS T3, 100 Å, 1.8 μm, 2.1×100 mm, Water Corporation, Milford, U.S.A) columns at 45° C.

Quaternary solvent system mobile phases were (A) 0.1% formic acid in water, (B) 0.1% formic acid in acetonitrile and (D) 100 mM ammonium formate, pH 3. Samples were separated on the HILIC column using the following gradient profile: a starting gradient of 95% B and 5% D was increased linearly to 70% A, 25% B and 5% D over a 5 min period at a 0.4 mL/min flow rate, followed by a 1 min isocratic gradient at 100% A at a 0.4 mL/min flow rate. For C18 separation, a chromatography gradient was performed as follows: starting conditions 100% A, with a linear increase to final conditions of 5% A, 95% B followed by an isocratic gradient at 95% B, 5% D for 1 min.

A binary pump was used for column regeneration and equilibration. The solvent system mobile phases were (A1) 100 mM ammonium formate, pH 3, (A2) 0.1% formic in 2-propanol, and (B1) 0.1% formic acid in acetonitrile. The HILIC column was stripped using 90% A2 for 5 min followed by a 2 min equilibration using 100% B1 at a 0.3 mL/min flowrate. Reverse phase C18 column regeneration was performed using 95% A1, 5% B1 for 2 min followed by column equilibration using 5% A1, 95% B1 for 5 min.

### Mass Spectrometry Data Acquisition

Mass spectrometry data was acquired using the ‘sensitivity’ mode in positive and negative electrospray ionization mode within 50-1200 Da range for primary metabolites and 100-2000 Da for complex lipids. For the electrospray acquisition, the capillary voltage was set at 1.5 kV (positive), 3.0 kV (negative), sample cone voltage 30V, source temperature at 120° C., cone gas flow 50 L/h and desolvation gas flow rate of 800 L/h with a scan time of 0.5 sec in continuum mode. Leucine Enkephalin; 556.2771 Da (positive) and 554.2615 Da (negative) was used for lockspray correction and scans were performed at 0.5 min. The injection volume for each sample was 3 μL, unless otherwise specified. The acquisition was carried out with instrument auto gain control to optimize instrument sensitivity over the sample acquisition time.

### Data Processing

Data were processed using Progenesis QI (Nonlinear, Waters). Peak picking and retention time alignment of LC-MS and MSe data were performed using the Progenesis QI software (Nonlinear, Waters). Data processing and peak annotations were performed using an in-house automated pipeline. Annotations were determined by matching accurate mass and retention times using customized libraries created from authentic standards and by matching experimental tandem mass spectrometry data against the NIST MSMS, LipidBlast or HMDB v3 theoretical fragmentations; for complex lipids retention time patterns characteristic of lipid subclasses was also considered. To correct for injection order drift, each feature was normalized using data from repeat injections of quality control samples collected every 10 injections throughout the run sequence. Measurement data were smoothed by Locally Weighted Scatterplot Smoothing (LOESS) signal correction (QC-RLSC) as previously described. Values are reported as ratios relative to the median of historical quality control reference samples run with every analytical batch for the given analyte.

### Assaying of CA125 and HE4

Serum CA125 and HE4 concentrations were measured using the Architect CA125II assay (Abbott Diagnostics, Abbott Park), and the HE4 EIA assay (Fujirebio Diagnostics, Malvern, PA). To calculate the ROMA score, a predictive index (PI) was calculated using serum HE4 and CA125 II levels and one of the following equations, depending on the patient's menopausal status:

$$1. Premenopausal : Predictive Index ( PI ) = - 12. + 2.38 * ln [ HE 4 ] + 0.0626 * ln [ CA 125 ]$$

$$2. Postmenopausal : Predictive Index ( PI ) = - 8.09 + 1.04 * ln [ HE 4 ] + 0.732 * ln [ CA 125 ]$$

The following equation used the predictive index (PI) for each patient to calculate a Risk of Ovarian Malignancy (ROMA) score:

$$ROMA score ( \% ) = \frac{exp ( PI )}{1 + exp ( PI )} * 100$$

### Example 3: Statistical Analysis

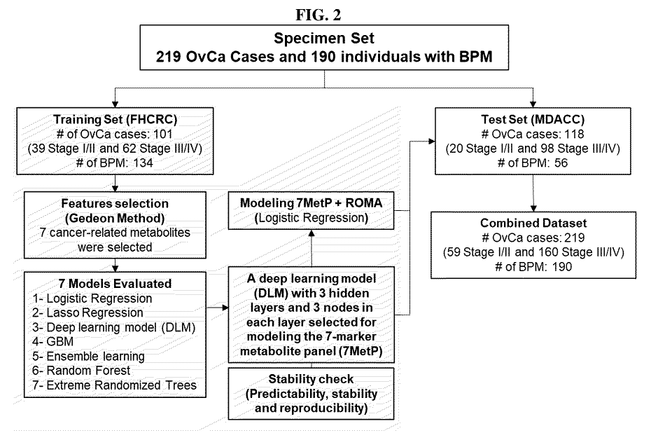

An overall schematic workflow of the study is provided in FIG. 2. Metabolite selection and model building was performed using metabolic profiles generated from serum samples from the FHCRC. The method reported by Gedeon was used to prioritize pertinent variables to be included in the model. This approach removes irrelevant or noisy variables by analyzing for the relative weight of each variable within the overall data matrix. An importance score is calculated by dividing the absolute value of the weight of an input connecting to an output by the total absolute value of all weights from that input. When applied in the deep learning model, this approach is recursively extended backwards through layers by taking the effect of a neuron on a connected node, then multiplying the derived weight by the effect of the given node on the target output and summing all connecting nodes.

$${P}_{jk} = \frac{\vert {w}_{jk} \vert}{{\sum }_{r = 1}^{nh} \vert {w}_{rk} \vert}$$

Here, Pⱼₖ represents the average contribution of a node j in a layer to a node k in the next layer. w is the weight on the connection and nh is the number of nodes in the next layer.

The contribution of an input neuron to an output is:

$${Q}_{ik} = {\sum }_{r = 1}^{nh} ( {P}_{ir} \times {P}_{rk} )$$

Using this approach, 20 iterations with slightly modified hyperparameters were introduced, and the relative variable importance score recalculated for each metabolite. Metabolites that consistently yielded a relative variable importance score>0.7 (corresponding to those metabolites with importance scores in the top 30th percentile) across all 20 iterations were selected to develop an algorithm for distinguishing early-stage OvCa from benign disease. Seven models, including deep learning, random forest, ensemble learning and gradient boosting method algorithms, incorporating the seven metabolites were assessed for distinguishing early-stage OvCa from benign disease. Performance of the models was evaluated using a 5-fold cross validation. To further evaluate model stability, perturbations (e.g., random selection and replacement) were introduced to the Training Set and the performance re-assessed.

A deep learning model (DLM) with 3 hidden layers and 3 nodes in each layer was selected for modeling the 7-marker metabolite panel (7MetP) based on AUC, and the 7MetP using fixed parameters tested for detection of OvCa in the MDACC cohort.

To assess the contributions of the 7MetP and ROMA, a logistic regression was first fitted with the 7MetP and ROMA as two separate predictors (Table 3). For ROMA, percentage risk was used as described above. Initial modeling was performed using early-stage OvCa cases and individuals with BPM from the FHCRC and testing of the model performed in the MDACC cohort.

To directly compare the performance of the combined 7MetP+ROMA model with ROMA, fixed risk thresholds of 11.4% in premenopausal women and 29.9% for postmenopausal were used, and positive predictive values (PPV), negative predictive values (NPV) as well as sensitivity and specificity estimates were calculated.

The combined score from the logistic regression model was converted to risk by exp(combined score)/(1+exp(combined score)).

Model discrimination was assessed based on receiver operating characteristic curve (ROC), as well as sensitivity and specificity estimates. The 95% confidence intervals (CI) for AUCs were estimated using the Delong method. P-values for specificity and sensitivity were estimated by calculating 2.5 and 97.5 percentiles of 1,000 boot straps on the delta values. All modeling was performed using the h2o package and R statistical program.

TABLE 3
Estimated coefficients for the combined model of the 7MetP plus ROMA.

| | Intercept | ROMA | 7MetP |
|---|---|---|---|
| Coefficients | −3.15 | 4.06 | 4.12 |

### Example 4: Cancer-Associated Metabolite Database

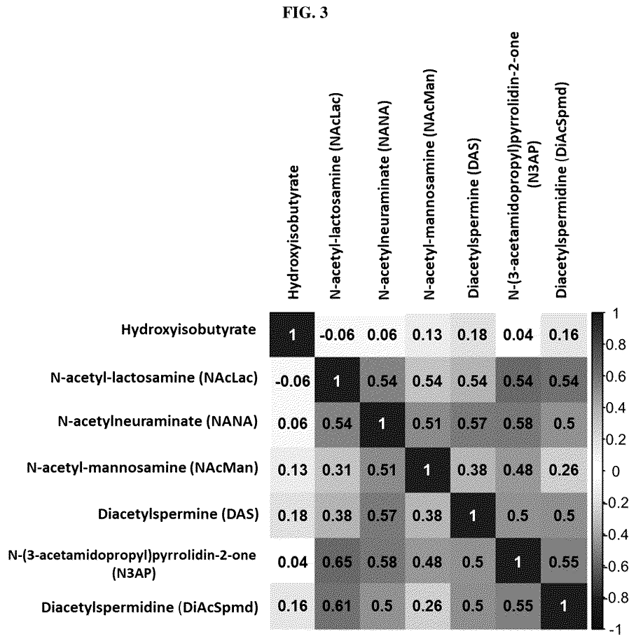

Untargeted metabolomics was conducted on a Training Set of sera from 101 OvCa cases (39 early stage and 62 late stage) and 134 subjects with BPM from the Fred Hutchinson Cancer Research Center (FHCRC) (Table 1). A total of 475 uniquely annotated metabolites were quantified (Table 4). To prioritize metabolites, relative importance scores were calculated using the Gedeon method, and metabolites were selected based on consistently exhibiting an importance score above 0.7. This approach resulted in seven metabolites being selected for model building, each of which have prior evidence for cancer relevance: diacetylspermine (DAS), diacetylspermidine (DiAcSpmd), N-(3-acetamidopropyl)pyrrolidin-2-one (N3AP), N-acetylneuraminate (NANA), N-acetyl-mannosamine (NAcMan), N-acetyl-lactosamine (NAcLac), and hydroxyisobutyric acid (HBA). Individual classifier performance of these metabolites for distinguishing OvCa cases from individuals with BPM ranged from 0.55 to 0.82 (Table 5; FIG. 3).

TABLE 4
Predictive performance of quantified metabolites for distinguishing OvCa from BPM in the Training Set.

| | | | | | | | | qvalue |
|---|---|---|---|---|---|---|---|---|
| Metabolite | AUC (95% CI) | | Sensitivity @ 99% sp | | Specificity @ 99% sn | | Pvalue | (Bonferroni) |
| | All cases (n = 101) versus BPM (n = 134) | | | | | | | |
| Diacetylspermine | 0.82 | (0.76-0.88) | 0.275 | (0.05-0.525) | 0.067 | (0.03-0.318) | 2.615E−17 | 1.240E−14 |
| Creatine riboside | 0.72 | (0.65-0.79) | 0.01 | (0-0.12) | 0.09 | (0-0.22) | 3.264E−09 | 1.550E−06 |
| ALPHA-HYDROXYISOBUTYRIC ACID | 0.71 | (0.64-0.77) | 0.09 | (0.015-0.165) | 0.164 | (0-0.231) | 3.624E−08 | 1.720E−05 |
| Acetylspermidine | 0.67 | (0.6-0.74) | 0.055 | (0-0.215) | 0.037 | (0.004-0.198) | 2.924E−06 | 1.389E−03 |
| Diacetylspermidine | 0.67 | (0.6-0.74) | 0.025 | (0-0.17) | 0.045 | (0.007-0.097) | 5.534E−06 | 2.629E−03 |
| N-ACETYL-L-ASPARTIC ACID | 0.65 | (0.57-0.72) | 0.2 | (0.11-0.33) | 0.067 | (0.004-0.19) | 6.181E−05 | 2.936E−02 |
| Monoelaidin | 0.64 | (0.57-0.72) | 0.05 | (0-0.145) | 0.067 | (0.03-0.131) | 9.086E−05 | 4.316E−02 |
| N-ALPHA-ACETYL-L-ASPARAGINE | 0.64 | (0.57-0.71) | 0.02 | (0-0.1) | 0.075 | (0.015-0.138) | 1.020E−04 | 4.845E−02 |
| 1-METHYLADENOSINE | 0.63 | (0.56-0.7) | 0.02 | (0-0.075) | 0.06 | (0.007-0.176) | 4.445E−04 | 2.111E−01 |
| 2-HYDROXYBUTYRIC ACID | 0.63 | (0.56-0.7) | 0.04 | (0.01-0.095) | 0.045 | (0.004-0.108) | 2.642E−04 | 1.255E−01 |
| N-ACETYLNEURAMINATE | 0.62 | (0.55-0.7) | 0.05 | (0.01-0.11) | 0.082 | (0-0.138) | 6.280E−04 | 2.983E−01 |
| HYPOXANTHINE | 0.62 | (0.55-0.69) | 0 | (0-0.005) | 0.037 | (0-0.138) | 9.646E−04 | 4.582E−01 |
| Heptanoylcarnitine | 0.62 | (0.55-0.69) | 0.05 | (0-0.183) | 0.03 | (0-0.082) | 8.392E−04 | 3.986E−01 |
| INOSINE | 0.62 | (0.54-0.69) | 0.08 | (0-0.15) | 0.022 | (0-0.15) | 1.277E−03 | 6.066E−01 |
| L-GLUTAMINE | 0.6 | (0.53-0.68) | 0.01 | (0-0.05) | 0.06 | (0.007-0.142) | 3.754E−03 | 1.000E+00 |
| DL-5-HYDROXYLYSINE | 0.6 | (0.53-0.68) | 0 | (0-0.12) | 0.007 | (0-0.067) | 3.798E−03 | 1.000E+00 |
| 5-OXO-D-PROLINE | 0.6 | (0.52-0.68) | 0.02 | (0-0.115) | 0.007 | (0-0.056) | 4.463E−03 | 1.000E+00 |
| Gabapentin | 0.59 | (0.52-0.67) | 0.01 | (0-0.085) | 0.007 | (0-0.06) | 7.894E−03 | 1.000E+00 |
| N-METHYL-L-GLUTAMATE | 0.59 | (0.52-0.67) | 0.01 | (0-0.08) | 0.015 | (0-0.101) | 7.521E−03 | 1.000E+00 |
| DECANOATE | 0.59 | (0.52-0.67) | 0.06 | (0.015-0.11) | 0.026 | (0-0.082) | 7.087E−03 | 1.000E+00 |
| Sphingomyelin(34:1) | 0.59 | (0.52-0.67) | 0.02 | (0-0.08) | 0.022 | (0-0.144) | 7.087E−03 | 1.000E+00 |
| Prostaglandin F2a | 0.59 | (0.52-0.66) | 0.01 | (0-0.095) | 0 | (0-0.026) | 9.207E−03 | 1.000E+00 |
| D-GLUCURONOLACTONE | 0.59 | (0.52-0.66) | 0 | (0-0.02) | 0.052 | (0-0.176) | 1.017E−02 | 1.000E+00 |
| TYRAMINE | 0.59 | (0.52-0.66) | 0.04 | (0-0.115) | 0.045 | (0-0.187) | 8.195E−03 | 1.000E+00 |
| Tyrosyl-Lysine | 0.59 | (0.51-0.66) | 0.06 | (0-0.11) | 0.022 | (0-0.123) | 1.229E−02 | 1.000E+00 |
| N-(3-acetamidopropyl)pyrrolidin-2-one | 0.58 | (0.51-0.66) | 0.03 | (0-0.075) | 0.037 | (0.011-0.146) | 1.656E−02 | 1.000E+00 |
| D-(+)-GALACTOSAMINE | 0.58 | (0.51-0.66) | 0.02 | (0-0.07) | 0 | (0-0.093) | 1.577E−02 | 1.000E+00 |
| Octopamine | 0.58 | (0.51-0.66) | 0.02 | (0-0.08) | 0.007 | (0-0.067) | 1.380E−02 | 1.000E+00 |
| 4-ACETAMIDOBUTANOATE | 0.58 | (0.51-0.66) | 0.02 | (0-0.065) | 0.03 | (0.007-0.071) | 1.524E−02 | 1.000E+00 |
| N-ACETYL-D-MANNOSAMINE | 0.58 | (0.5-0.65) | 0.04 | (0.01-0.105) | 0.03 | (0-0.139) | 2.044E−02 | 1.000E+00 |
| DEOXYURIDINE | 0.58 | (0.5-0.65) | 0.02 | (0-0.06) | 0.022 | (0-0.082) | 2.394E−02 | 1.000E+00 |
| ADENOSINE | 0.58 | (0.5-0.65) | 0.03 | (0-0.15) | 0.015 | (0-0.116) | 2.015E−02 | 1.000E+00 |
| GLYCEROL 2-PHOSPHATE | 0.58 | (0.5-0.65) | 0 | (0-0.09) | 0.007 | (0-0.071) | 2.483E−02 | 1.000E+00 |
| 3-METHOXYTYRAMINE | 0.58 | (0.5-0.65) | 0.02 | (0-0.07) | 0.015 | (0-0.056) | 2.015E−02 | 1.000E+00 |
| ALPHA-D-GLUCOSE | 0.57 | (0.5-0.65) | 0.03 | (0-0.09) | 0 | (0-0.101) | 2.540E−02 | 1.000E+00 |
| CITRULLINE | 0.57 | (0.5-0.65) | 0.01 | (0-0.1) | 0 | (0-0.067) | 3.026E−02 | 1.000E+00 |
| S-HEXYL-GLUTATHIONE | 0.57 | (0.5-0.65) | 0.05 | (0.01-0.13) | 0.03 | (0-0.082) | 2.646E−02 | 1.000E+00 |
| D-TRYPTOPHAN | 0.57 | (0.5-0.65) | 0.04 | (0-0.09) | 0.037 | (0-0.086) | 2.882E−02 | 1.000E+00 |
| BETAINE | 0.57 | (0.5-0.65) | 0.02 | (0-0.08) | 0.007 | (0-0.037) | 2.506E−02 | 1.000E+00 |
| NG, NG-dimethyl-L-arginine | 0.57 | (0.49-0.64) | 0.03 | (0-0.075) | 0.015 | (0-0.045) | 4.445E−02 | 1.000E+00 |
| FORMYL-L-METHIONYL PEPTIDE | 0.57 | (0.49-0.64) | 0.06 | (0-0.16) | 0.022 | (0-0.082) | 4.390E−02 | 1.000E+00 |
| DOPAMINE | 0.57 | (0.49-0.64) | 0.03 | (0-0.11) | 0.06 | (0.015-0.146) | 3.856E−02 | 1.000E+00 |
| RETINYL PALMITATE | 0.57 | (0.49-0.64) | 0.01 | (0-0.05) | 0.037 | (0-0.105) | 4.408E−02 | 1.000E+00 |
| 2.3-DIPHOSPHO-D-GLYCERIC ACID | 0.57 | (0.49-0.64) | 0.01 | (0-0.035) | 0.06 | (0-0.131) | 4.142E−02 | 1.000E+00 |
| D-GALACTOSE | 0.56 | (0.49-0.64) | 0.06 | (0.01-0.125) | 0.015 | (0-0.049) | 5.002E−02 | 1.000E+00 |
| 2.4-DIHYDROXYPYRIMIDINE-5- | 0.56 | (0.49-0.64) | 0.05 | (0-0.1) | 0.037 | (0-0.082) | 5.022E−02 | 1.000E+00 |
| CARBOXYLIC ACID | | | | | | | | |
| Acylcarnitine(C14:0) | 0.56 | (0.49-0.64) | 0.02 | (0-0.05) | 0.075 | (0.033-0.157) | 4.784E−02 | 1.000E+00 |
| METHYL VANILLATE | 0.56 | (0.49-0.63) | 0.02 | (0-0.065) | 0.108 | (0.067-0.157) | 5.592E−02 | 1.000E+00 |
| L-CYSTINE | 0.56 | (0.48-0.63) | 0.04 | (0.01-0.115) | 0 | (0-0.067) | 7.498E−02 | 1.000E+00 |
| L-CYSTEINE_In Source | 0.56 | (0.48-0.63) | 0.05 | (0.01-0.09) | 0.007 | (0-0.049) | 7.388E−02 | 1.000E+00 |
| N6-(DELTA2-ISOPENTENYL)- | 0.56 | (0.48-0.63) | 0 | (0-0.105) | 0.015 | (0-0.067) | 6.454E−02 | 1.000E+00 |
| ADENINE | | | | | | | | |
| 6-CARBOXYHEXANOATE | 0.56 | (0.48-0.63) | 0.01 | (0-0.045) | 0.075 | (0.033-0.123) | 7.306E−02 | 1.000E+00 |
| D-PANTOTHENIC ACID_peak 2 | 0.56 | (0.48-0.63) | 0 | (0-0.075) | 0 | (0-0.034) | 6.503E−02 | 1.000E+00 |
| N-acetyllactosamine | 0.55 | (0.48-0.63) | 0.03 | (0-0.085) | 0.045 | (0.007-0.093) | 9.074E−02 | 1.000E+00 |
| 3-(2- | 0.55 | (0.48-0.63) | 0 | (0-0.04) | 0.022 | (0-0.123) | 8.697E−02 | 1.000E+00 |
| HYDROXYPHENYL)PROPANOATE | | | | | | | | |
| 3-METHYLHISTAMINE | 0.55 | (0.48-0.63) | 0.08 | (0.03-0.165) | 0.052 | (0.022-0.101) | 8.154E−02 | 1.000E+00 |
| Benzofuran | 0.55 | (0.48-0.63) | 0 | (0-0.03) | 0.015 | (0-0.101) | 9.138E−02 | 1.000E+00 |
| 2-Methylbutyroylcarnitine | 0.55 | (0.48-0.63) | 0.03 | (0.01-0.07) | 0 | (0-0.079) | 9.074E−02 | 1.000E+00 |
| SEROTONIN | 0.55 | (0.48-0.62) | 0 | (0-0) | 0.03 | (0.007-0.093) | 9.562E−02 | 1.000E+00 |
| 3-HYDROXYANTHRANILATE | 0.55 | (0.48-0.62) | 0 | (0-0.07) | 0.052 | (0.015-0.134) | 9.695E−02 | 1.000E+00 |
| S-(5′-ADENOSYL)-L-METHIONINE | 0.55 | (0.47-0.62) | 0 | (0-0.06) | 0.019 | (0-0.075) | 1.007E−01 | 1.000E+00 |
| 2-AMINO-2-METHYLPROPANOATE | 0.55 | (0.47-0.62) | 0.01 | (0-0.085) | 0.015 | (0-0.071) | 1.136E−01 | 1.000E+00 |
| 2-DEOXY-D-GLUCOSE | 0.55 | (0.47-0.62) | 0.01 | (0-0.07) | 0.026 | (0-0.082) | 1.121E−01 | 1.000E+00 |
| PHENOL | 0.54 | (0.47-0.62) | 0 | (0-0.05) | 0.082 | (0.03-0.146) | 1.413E−01 | 1.000E+00 |
| BIOTIN | 0.54 | (0.47-0.62) | 0.05 | (0.015-0.13) | 0.037 | (0.007-0.112) | 1.294E−01 | 1.000E+00 |
| PHENETHYLAMINE | 0.54 | (0.47-0.62) | 0.03 | (0-0.08) | 0.03 | (0.007-0.06) | 1.413E−01 | 1.000E+00 |
| SPHINGANINE | 0.54 | (0.47-0.62) | 0.06 | (0.01-0.13) | 0.052 | (0.015-0.116) | 1.298E−01 | 1.000E+00 |
| GALACTITOL | 0.54 | (0.46-0.62) | 0 | (0-0.045) | 0.03 | (0.007-0.075) | 1.543E−01 | 1.000E+00 |
| MALTOSE | 0.54 | (0.46-0.62) | 0.03 | (0-0.08) | 0.019 | (0-0.056) | 1.448E−01 | 1.000E+00 |
| Trazodone | 0.54 | (0.46-0.62) | 0.08 | (0.005-0.185) | 0 | (0-0.067) | 1.378E−01 | 1.000E+00 |
| DETHIOBIOTIN | 0.54 | (0.46-0.61) | 0 | (0-0.075) | 0.052 | (0.015-0.134) | 1.721E−01 | 1.000E+00 |
| 5(s)-HETE | 0.54 | (0.46-0.61) | 0 | (0-0.025) | 0.037 | (0-0.075) | 1.777E−01 | 1.000E+00 |
| 4-PYRIDOXATE | 0.54 | (0.46-0.61) | 0 | (0-0) | 0.037 | (0.007-0.09) | 1.687E−01 | 1.000E+00 |
| BENZALDEHYDE | 0.54 | (0.46-0.61) | 0 | (0-0.116) | 0.007 | (0-0.067) | 1.581E−01 | 1.000E+00 |
| Monoethylhexyl phthalic acid | 0.54 | (0.46-0.61) | 0 | (0-0.075) | 0.037 | (0-0.112) | 1.590E−01 | 1.000E+00 |
| KYNURENINE | 0.54 | (0.46-0.61) | 0 | (0-0.03) | 0.034 | (0.007-0.09) | 1.557E−01 | 1.000E+00 |
| URATE | 0.54 | (0.46-0.61) | 0.02 | (0-0.075) | 0.052 | (0.011-0.138) | 1.726E−01 | 1.000E+00 |
| L-GLUTAMIC ACID | 0.53 | (0.46-0.61) | 0 | (0-0.035) | 0.037 | (0-0.086) | 1.906E−01 | 1.000E+00 |
| Hexaethylene glycol_contaminant | 0.53 | (0.46-0.61) | 0.02 | (0-0.055) | 0.03 | (0.007-0.071) | 2.149E−01 | 1.000E+00 |
| CORTISOL | 0.53 | (0.46-0.61) | 0 | (0-0.06) | 0.015 | (0-0.097) | 1.955E−01 | 1.000E+00 |
| L-RHAMNOSE | 0.53 | (0.46-0.61) | 0 | (0-0.056) | 0.09 | (0-0.142) | 2.132E−01 | 1.000E+00 |
| THIAMINE | 0.53 | (0.46-0.61) | 0.01 | (0-0.055) | 0.037 | (0.007-0.108) | 1.849E−01 | 1.000E+00 |
| L-2-PHOSPHOGLYCERIC | 0.53 | (0.46-0.61) | 0.04 | (0.01-0.09) | 0.067 | (0.018-0.116) | 1.938E−01 | 1.000E+00 |
| ACID_breakthrough | | | | | | | | |
| INDOLE-3-ACETALDEHYDE | 0.53 | (0.46-0.61) | 0.01 | (0-0.075) | 0 | (0-0.026) | 2.132E−01 | 1.000E+00 |
| 5′-DEOXYADENOSINE | 0.53 | (0.46-0.61) | 0 | (0-0.03) | 0.03 | (0-0.082) | 1.870E−01 | 1.000E+00 |
| 5′-METHYLTHIOADENOSINE | 0.53 | (0.46-0.61) | 0.02 | (0-0.06) | 0.004 | (0-0.064) | 1.880E−01 | 1.000E+00 |
| D-(−)-3-PHOSPHOGLYCERIC ACID | 0.53 | (0.46-0.6) | 0.02 | (0-0.07) | 0.015 | (0-0.041) | 2.160E−01 | 1.000E+00 |
| 3-DEHYDROSHIKIMATE | 0.53 | (0.46-0.6) | 0.01 | (0-0.06) | 0.045 | (0-0.116) | 2.160E−01 | 1.000E+00 |
| N-ACETYL-L-ALANINE | 0.53 | (0.45-0.6) | 0 | (0-0.015) | 0.007 | (0-0.03) | 2.451E−01 | 1.000E+00 |
| Prostaglandin E2 | 0.53 | (0.45-0.6) | 0.01 | (0-0.06) | 0.022 | (0-0.116) | 2.282E−01 | 1.000E+00 |
| D-PANTOTHENIC ACID | 0.53 | (0.45-0.6) | 0 | (0-0.07) | 0.026 | (0-0.101) | 2.390E−01 | 1.000E+00 |
| 2′-DEOXYGUANOSINE 5′- | 0.53 | (0.45-0.6) | 0.03 | (0-0.07) | 0.037 | (0-0.127) | 2.183E−01 | 1.000E+00 |
| MONOPHOSPHATE | | | | | | | | |
| ADENOSINE 5′-MONOPHOSPHATE | 0.53 | (0.45-0.6) | 0.04 | (0-0.125) | 0.034 | (0-0.098) | 2.206E−01 | 1.000E+00 |
| EPINEPHRINE | 0.53 | (0.45-0.6) | 0.035 | (0.01-0.075) | 0.015 | (0-0.056) | 2.426E−01 | 1.000E+00 |
| 6-HYDROXYNICOTINATE | 0.53 | (0.45-0.6) | 0.01 | (0-0.065) | 0.06 | (0.015-0.138) | 2.519E−01 | 1.000E+00 |
| 2-Phenylethanol glucuronide | 0.53 | (0.45-0.6) | 0 | (0-0.08) | 0.015 | (0-0.067) | 2.384E−01 | 1.000E+00 |
| Sphingomyelin(36:1) | 0.53 | (0.45-0.6) | 0.02 | (0-0.07) | 0.045 | (0.007-0.116) | 2.172E−01 | 1.000E+00 |
| 3-HYDROXY-3-METHYLGLUTARATE | 0.53 | (0.45-0.6) | 0 | (0-0.09) | 0.052 | (0.015-0.135) | 2.206E−01 | 1.000E+00 |
| 11-Hydroxy-9-tridecenoic acid | 0.53 | (0.45-0.6) | 0.01 | (0-0.08) | 0.026 | (0.007-0.064) | 2.259E−01 | 1.000E+00 |
| Acylcarnitine(C16:0) | 0.53 | (0.45-0.6) | 0 | (0-0.04) | 0.022 | (0-0.075) | 2.384E−01 | 1.000E+00 |
| 1-AMINOCYCLOPROPANE-1- | 0.52 | (0.45-0.6) | 0.03 | (0.01-0.075) | 0.041 | (0.015-0.071) | 2.761E−01 | 1.000E+00 |
| CARBOXYLATE | | | | | | | | |
| GLUCONIC ACID | 0.52 | (0.45-0.6) | 0.01 | (0-0.065) | 0 | (0-0.082) | 2.907E−01 | 1.000E+00 |
| URIDINE-5-MONOPHOSPHATE | 0.52 | (0.45-0.6) | 0.02 | (0-0.07) | 0.022 | (0-0.075) | 2.594E−01 | 1.000E+00 |
| Phosphatidylethanolamine(34:1) | 0.52 | (0.45-0.6) | 0 | (0-0.02) | 0.045 | (0.018-0.09) | 2.607E−01 | 1.000E+00 |
| N-ACETYL-L-LEUCINE | 0.52 | (0.45-0.6) | 0 | (0-0.01) | 0.007 | (0-0.157) | 2.619E−01 | 1.000E+00 |
| GUANOSINE 5′-MONOPHOSPHATE | 0.52 | (0.45-0.6) | 0.03 | (0-0.07) | 0.037 | (0-0.101) | 2.867E−01 | 1.000E+00 |
| EDTA_exogenous | 0.52 | (0.45-0.6) | 0.02 | (0-0.06) | 0.052 | (0.018-0.116) | 2.607E−01 | 1.000E+00 |
| 6-keto Prostaglandin F1a | 0.52 | (0.45-0.6) | 0.04 | (0-0.11) | 0.045 | (0-0.09) | 2.735E−01 | 1.000E+00 |
| 3-METHYGLUTARIC ACID | 0.52 | (0.45-0.6) | 0.01 | (0-0.07) | 0.022 | (0-0.071) | 2.940E−01 | 1.000E+00 |
| TAURINE | 0.52 | (0.45-0.6) | 0.03 | (0-0.11) | 0.03 | (0.004-0.09) | 2.683E−01 | 1.000E+00 |
| L-PHENYLALANINE | 0.52 | (0.45-0.6) | 0.01 | (0-0.075) | 0.037 | (0.015-0.075) | 2.761E−01 | 1.000E+00 |
| Hydroxybutyrylcarnitine | 0.52 | (0.45-0.6) | 0.01 | (0-0.08) | 0 | (0-0.037) | 2.619E−01 | 1.000E+00 |
| Hexanoic Acid | 0.52 | (0.45-0.6) | 0 | (0-0.035) | 0.03 | (0-0.075) | 2.677E−01 | 1.000E+00 |
| cis-Quinceoxepane [M + Na]+ | 0.52 | (0.44-0.6) | 0.005 | (0-0.08) | 0 | (0-0.026) | 3.083E−01 | 1.000E+00 |
| PYRIDOXINE | 0.52 | (0.44-0.59) | 0.02 | (0-0.05) | 0 | (0-0.105) | 3.427E−01 | 1.000E+00 |
| RIBOFLAVIN | 0.52 | (0.44-0.59) | 0 | (0-0.025) | 0.06 | (0.015-0.119) | 3.090E−01 | 1.000E+00 |
| Glutarylcarnitine [M − H2O + H]+ | 0.52 | (0.44-0.59) | 0 | (0-0.02) | 0 | (0-0.052) | 3.271E−01 | 1.000E+00 |
| Free Fatty Acid (14:1) (myristoleic acid) | 0.52 | (0.44-0.59) | 0.01 | (0-0.04) | 0.022 | (0-0.068) | 3.463E−01 | 1.000E+00 |
| GUANOSINE 5′-DIPHOSPHATE | 0.52 | (0.44-0.59) | 0 | (0-0.03) | 0.086 | (0.045-0.161) | 3.194E−01 | 1.000E+00 |
| L-SERINE | 0.52 | (0.44-0.59) | 0.01 | (0-0.055) | 0.037 | (0.007-0.101) | 3.449E−01 | 1.000E+00 |
| CREATININE | 0.52 | (0.44-0.59) | 0.01 | (0-0.045) | 0.045 | (0-0.157) | 3.427E−01 | 1.000E+00 |
| L-CYSTEINE | 0.52 | (0.44-0.59) | 0.01 | (0-0.065) | 0.007 | (0-0.112) | 3.420E−01 | 1.000E+00 |
| XANTHINE | 0.52 | (0.44-0.59) | 0.04 | (0-0.1) | 0.112 | (0.03-0.183) | 3.349E−01 | 1.000E+00 |
| ALLANTOIN | 0.52 | (0.44-0.59) | 0.04 | (0.01-0.09) | 0.03 | (0-0.075) | 3.406E−01 | 1.000E+00 |
| Acylcarnitine(C6:0) | 0.52 | (0.44-0.59) | 0.01 | (0-0.06) | 0.022 | (0-0.064) | 3.463E−01 | 1.000E+00 |
| INOSINE 5′-MONOPHOSPHATE | 0.51 | (0.44-0.59) | 0.01 | (0-0.03) | 0.052 | (0-0.142) | 3.785E−01 | 1.000E+00 |
| CYTIDINE 5′-DIPHOSPHOCHOLINE | 0.51 | (0.44-0.59) | 0 | (0-0.025) | 0.037 | (0-0.127) | 3.711E−01 | 1.000E+00 |
| Acetylcarnitine | 0.51 | (0.44-0.59) | 0.005 | (0-0.055) | 0 | (0-0.037) | 3.550E−01 | 1.000E+00 |
| N-ACETYL-L-PHENYLALANINE | 0.51 | (0.44-0.59) | 0 | (0-0.01) | 0.022 | (0.007-0.079) | 3.696E−01 | 1.000E+00 |
| STACHYOSE | 0.51 | (0.44-0.59) | 0.03 | (0-0.095) | 0.037 | (0.007-0.119) | 3.521E−01 | 1.000E+00 |
| THYROTROPIN RELEASING | 0.51 | (0.44-0.59) | 0 | (0-0.045) | 0 | (0-0.03) | 3.682E−01 | 1.000E+00 |
| HORMONE | | | | | | | | |
| TAUROLITHOCHOLATE | 0.51 | (0.44-0.59) | 0.01 | (0-0.04) | 0.03 | (0-0.123) | 3.557E−01 | 1.000E+00 |
| ADENOSINE 3′.5′-DIPHOSPHATE | 0.51 | (0.44-0.59) | 0 | (0-0.07) | 0.03 | (0.007-0.082) | 3.485E−01 | 1.000E+00 |
| 2-HYDROXYPHENYLACETIC ACID | 0.51 | (0.44-0.58) | 0.01 | (0-0.035) | 0.063 | (0.03-0.119) | 3.980E−01 | 1.000E+00 |
| N-FORMYLGLYCINE | 0.51 | (0.43-0.59) | 0.02 | (0-0.095) | 0.022 | (0-0.105) | 3.934E−01 | 1.000E+00 |
| THYROXINE | 0.51 | (0.43-0.59) | 0 | (0-0.07) | 0.007 | (0-0.064) | 4.078E−01 | 1.000E+00 |
| Chavicol O-beta-glucopyranoside | 0.51 | (0.43-0.59) | 0 | (0-0.045) | 0 | (0-0.026) | 3.957E−01 | 1.000E+00 |
| [M − H2O + H]+ | | | | | | | | |
| N-ACETYL-D-TRYPTOPHAN | 0.51 | (0.43-0.58) | 0.01 | (0-0.04) | 0.06 | (0.015-0.138) | 4.314E−01 | 1.000E+00 |
| XANTHOSINE 5′-MONOPHOSPHATE | 0.51 | (0.43-0.58) | 0.07 | (0.015-0.12) | 0.022 | (0-0.068) | 4.414E−01 | 1.000E+00 |
| MEVALOLACTONE | 0.51 | (0.43-0.58) | 0 | (0-0.01) | 0.045 | (0.015-0.123) | 4.368E−01 | 1.000E+00 |
| 5-VALEROLACTONE | 0.51 | (0.43-0.58) | 0 | (0-0.05) | 0 | (0-0.022) | 4.230E−01 | 1.000E+00 |
| TRYPTOPHAN_In Source | 0.51 | (0.43-0.58) | 0.01 | (0-0.05) | 0.015 | (0-0.064) | 4.368E−01 | 1.000E+00 |
| ETHYLMALONIC ACID | 0.51 | (0.43-0.58) | 0.02 | (0-0.04) | 0.022 | (0-0.093) | 4.070E−01 | 1.000E+00 |
| NICOTINAMIDE MONONUCLEOTIDE | 0.51 | (0.43-0.58) | 0.01 | (0-0.04) | 0 | (0-0.079) | 4.330E−01 | 1.000E+00 |
| N-Undecanoylglycine | 0.51 | (0.43-0.58) | 0.01 | (0-0.03) | 0 | (0-0.071) | 4.192E−01 | 1.000E+00 |
| ADIPIC ACID | 0.51 | (0.43-0.58) | 0 | (0-0.015) | 0 | (0-0.052) | 4.245E−01 | 1.000E+00 |
| 2′-DEOXYGUANOSINE 5′- | 0.51 | (0.43-0.58) | 0 | (0-0.03) | 0.022 | (0-0.071) | 4.368E−01 | 1.000E+00 |
| TRIPHOSPHATE | | | | | | | | |
| D-(+)-GALACTURONIC ACID | 0.5 | (0.43-0.58) | 0.01 | (0-0.04) | 0.022 | (0.007-0.071) | 4.638E−01 | 1.000E+00 |
| GUANOSINE 3′.5′-CYCLIC | 0.5 | (0.43-0.58) | 0 | (0-0.08) | 0.041 | (0.015-0.082) | 4.646E−01 | 1.000E+00 |
| MONOPHOSPHATE | | | | | | | | |
| L-TYROSINE | 0.5 | (0.43-0.58) | 0.01 | (0-0.04) | 0.037 | (0-0.086) | 4.654E−01 | 1.000E+00 |
| N-ACETYL-DL-SERINE | 0.5 | (0.43-0.58) | 0.01 | (0-0.05) | 0.022 | (0-0.052) | 4.623E−01 | 1.000E+00 |
| LEUCINE | 0.5 | (0.43-0.58) | 0.005 | (0-0.05) | 0.03 | (0.007-0.064) | 4.747E−01 | 1.000E+00 |
| OROTATE | 0.5 | (0.43-0.58) | 0.04 | (0.005-0.105) | 0.015 | (0-0.056) | 4.662E−01 | 1.000E+00 |
| 5.6-DIHYDROURACIL | 0.5 | (0.43-0.58) | 0 | (0-0.06) | 0.06 | (0.015-0.142) | 4.538E−01 | 1.000E+00 |
| URIDINE | 0.5 | (0.43-0.58) | 0.01 | (0-0.055) | 0.022 | (0-0.064) | 4.561E−01 | 1.000E+00 |
| nicotinate beta-D-ribonucleotide | 0.5 | (0.43-0.58) | 0 | (0-0.02) | 0.007 | (0-0.06) | 4.638E−01 | 1.000E+00 |
| Lysophosphatidylcholine(20:0) | 0.5 | (0.43-0.58) | 0.04 | (0.01-0.095) | 0.007 | (0-0.052) | 4.879E−01 | 1.000E+00 |
| 1-OLEOYL-RAC-GLYCEROL | 0.5 | (0.43-0.58) | 0.01 | (0-0.04) | 0.037 | (0-0.097) | 4.491E−01 | 1.000E+00 |
| 2-aminobenzamide | 0.5 | (0.43-0.58) | 0.03 | (0.005-0.085) | 0.037 | (0.011-0.082) | 4.600E−01 | 1.000E+00 |
| BILIVERDIN | 0.5 | (0.43-0.58) | 0.01 | (0-0.09) | 0.022 | (0.004-0.102) | 4.491E−01 | 1.000E+00 |
| ADENOSINE 5′-DIPHOSPHORIBOSE | 0.5 | (0.42-0.58) | 0.02 | (0-0.06) | 0 | (0-0.022) | 4.988E−01 | 1.000E+00 |
| AZELAIC ACID | 0.5 | (0.42-0.57) | 0 | (0-0.03) | 0.022 | (0-0.06) | 4.514E−01 | 1.000E+00 |
| ROSMARINIC ACID | 0.5 | (0.42-0.57) | 0 | (0-0.04) | 0.022 | (0-0.064) | 4.484E−01 | 1.000E+00 |
| DEOXYCYTIDINE | 0.5 | (0.42-0.57) | 0.04 | (0.01-0.09) | 0.022 | (0-0.097) | 4.646E−01 | 1.000E+00 |
| Indoleacetaldehyde | 0.5 | (0.42-0.57) | 0 | (0-0.02) | 0.045 | (0-0.104) | 4.646E−01 | 1.000E+00 |
| HYPOTAURINE_second peak | 0.5 | (0.42-0.57) | 0.02 | (0-0.065) | 0.052 | (0.015-0.112) | 4.716E−01 | 1.000E+00 |
| 3-HYDROXYBENZYL ALCOHOL | 0.5 | (0.42-0.57) | 0.01 | (0-0.05) | 0 | (0-0.041) | 4.638E−01 | 1.000E+00 |
| 3-Dehydrocarnitine | 0.5 | (0.42-0.57) | 0.01 | (0-0.06) | 0.011 | (0-0.041) | 4.817E−01 | 1.000E+00 |
| NORADRENALINE | 0.5 | (0.42-0.57) | 0 | (0-0.055) | 0 | (0-0.045) | 4.623E−01 | 1.000E+00 |
| S-CARBOXYMETHYL-L-CYSTEINE | 0.5 | (0.42-0.57) | 0.01 | (0-0.03) | 0.022 | (0-0.079) | 4.561E−01 | 1.000E+00 |
| ISOCITRIC ACID | 0.5 | (0.42-0.57) | 0.01 | (0-0.055) | 0.06 | (0.015-0.119) | 4.895E−01 | 1.000E+00 |
| ALPHA-AMINOADIPATE | 0.5 | (0.42-0.57) | 0 | (0-0) | 0.007 | (0-0.049) | 4.887E−01 | 1.000E+00 |
| Acylcarnitine(C18:2n-6) | 0.5 | (0.42-0.57) | 0 | (0-0.035) | 0.045 | (0.015-0.112) | 4.778E−01 | 1.000E+00 |
| Acylcarnitine(C12:0) | 0.5 | (0.42-0.57) | 0 | (0-0.03) | 0.022 | (0-0.071) | 4.538E−01 | 1.000E+00 |
| 8-Deoxy-11,13-dihydroxygrosheimin | 0.5 | (0.42-0.57) | 0 | (0-0.05) | 0.015 | (0-0.067) | 4.732E−01 | 1.000E+00 |
| 2′-DEOXYURIDINE 5′-MONO-PHOS- | 0.5 | (0.42-0.57) | 0 | (0-0) | 0.015 | (0-0.06) | 4.499E−01 | 1.000E+00 |
| PHATE | | | | | | | | |
| PYRIDOXAL 5′-PHOSPHATE | 0.49 | (0.42-0.57) | 0.015 | (0-0.065) | 0.019 | (0-0.086) | 4.131E−01 | 1.000E+00 |
| L-PIPECOLIC ACID | 0.49 | (0.42-0.57) | 0 | (0-0) | 0 | (0-0.015) | 4.399E−01 | 1.000E+00 |
| D-RIBULOSE 1.5-BISPHOSPHATE | 0.49 | (0.42-0.57) | 0.02 | (0-0.07) | 0.037 | (0.004-0.097) | 4.284E−01 | 1.000E+00 |
| URIDINE 5′-DIPHOSPHOGLUCURONIC | 0.49 | (0.42-0.57) | 0.02 | (0-0.085) | 0.03 | (0-0.09) | 4.353E−01 | 1.000E+00 |
| ACID | | | | | | | | |
| LUMICHROME | 0.49 | (0.42-0.57) | 0.03 | (0-0.08) | 0 | (0-0.022) | 4.468E−01 | 1.000E+00 |
| N-ACETYL-DL-METHIONINE | 0.49 | (0.42-0.57) | 0.01 | (0-0.075) | 0.007 | (0-0.052) | 4.261E−01 | 1.000E+00 |
| OMEGA-HYDROXYDODECANOIC | 0.49 | (0.42-0.57) | 0 | (0-0.025) | 0 | (0-0.019) | 4.430E−01 | 1.000E+00 |
| ACID | | | | | | | | |
| GLYCERALDEHYDE | 0.49 | (0.42-0.57) | 0 | (0-0.02) | 0 | (0-0.056) | 4.399E−01 | 1.000E+00 |
| Acetylsalicyclic acid | 0.49 | (0.42-0.57) | 0 | (0-0.06) | 0.019 | (0-0.068) | 4.453E−01 | 1.000E+00 |
| 2,6-diisopropylnapthalene [M + Na]+ | 0.49 | (0.42-0.57) | 0 | (0-0.02) | 0 | (0-0.03) | 4.406E−01 | 1.000E+00 |
| 1-(sn-Glycero-3-phosphate)-1D-myo- | 0.49 | (0.42-0.57) | 0 | (0-0) | 0.082 | (0.015-0.153) | 4.414E−01 | 1.000E+00 |
| inositol | | | | | | | | |
| PHENYLETHANOLAMINE | 0.49 | (0.42-0.56) | 0.02 | (0-0.07) | 0 | (0-0.079) | 3.972E−01 | 1.000E+00 |
| Lauroylcarnitine_break through | 0.49 | (0.42-0.56) | 0.01 | (0-0.055) | 0.045 | (0.007-0.093) | 4.002E−01 | 1.000E+00 |
| Free Fatty Acid (16:0) (palmitic acid) | 0.49 | (0.41-0.57) | 0.01 | (0-0.035) | 0 | (0-0.011) | 4.078E−01 | 1.000E+00 |
| HYPOTAURINE | 0.49 | (0.41-0.56) | 0.02 | (0-0.06) | 0.015 | (0-0.052) | 3.630E−01 | 1.000E+00 |
| THYMIDINE | 0.49 | (0.41-0.56) | 0 | (0-0.035) | 0.015 | (0-0.068) | 3.492E−01 | 1.000E+00 |
| PYRIDOXAL | 0.49 | (0.41-0.56) | 0.04 | (0-0.095) | 0.03 | (0.007-0.064) | 3.601E−01 | 1.000E+00 |
| ALPHA-KETOGLUTARIC ACID | 0.49 | (0.41-0.56) | 0 | (0-0.07) | 0.045 | (0.015-0.127) | 3.514E−01 | 1.000E+00 |
| O-SUCCINYL-L-HOMOSERINE | 0.49 | (0.41-0.56) | 0.045 | (0-0.11) | 0.015 | (0-0.09) | 3.792E−01 | 1.000E+00 |
| O-ACETYL-L-CARNITINE | 0.49 | (0.41-0.56) | 0 | (0-0.01) | 0.022 | (0-0.06) | 3.652E−01 | 1.000E+00 |
| 3.4-DIHYDROXYPHENYL GLYCOL | 0.49 | (0.41-0.56) | 0.03 | (0-0.08) | 0.034 | (0-0.09) | 3.741E−01 | 1.000E+00 |
| N′-nitrosonornicotine | 0.49 | (0.41-0.56) | 0 | (0-0.03) | 0.015 | (0-0.064) | 3.852E−01 | 1.000E+00 |
| 4-Acetamido-2-aminobutanoic acid | 0.48 | (0.41-0.56) | 0.02 | (0-0.07) | 0.007 | (0-0.071) | 3.243E−01 | 1.000E+00 |
| sterol | 0.48 | (0.41-0.56) | 0.01 | (0-0.05) | 0.007 | (0-0.056) | 3.442E−01 | 1.000E+00 |
| INDOLE-3-PYRUVIC ACID | 0.48 | (0.41-0.56) | 0 | (0-0.04) | 0.007 | (0-0.079) | 3.229E−01 | 1.000E+00 |
| Octanoic Acid | 0.48 | (0.41-0.56) | 0 | (0-0.04) | 0.037 | (0.007-0.079) | 3.208E−01 | 1.000E+00 |
| ASCORBATE | 0.48 | (0.41-0.56) | 0.02 | (0-0.05) | 0.022 | (0-0.075) | 3.313E−01 | 1.000E+00 |
| N1-ACETYLSPERMINE | 0.48 | (0.41-0.56) | 0.01 | (0-0.04) | 0.037 | (0.007-0.093) | 3.456E−01 | 1.000E+00 |
| Propionylcarnitine | 0.48 | (0.41-0.56) | 0 | (0-0.025) | 0 | (0-0.067) | 3.131E−01 | 1.000E+00 |
| L-TRYPTOPHANAMIDE | 0.48 | (0.41-0.56) | 0.04 | (0.01-0.075) | 0.052 | (0.015-0.131) | 3.250E−01 | 1.000E+00 |
| GUANINE | 0.48 | (0.41-0.56) | 0 | (0-0.025) | 0.022 | (0-0.052) | 3.076E−01 | 1.000E+00 |
| D-2-Hydroxyglutarate | 0.48 | (0.41-0.56) | 0.01 | (0-0.04) | 0.022 | (0-0.06) | 3.110E−01 | 1.000E+00 |
| CORTISOL 21-ACETATE | 0.48 | (0.4-0.56) | 0 | (0-0.03) | 0 | (0-0.015) | 3.021E−01 | 1.000E+00 |
| 3.4-DIHYDROXYBENZOATE | 0.48 | (0.4-0.56) | 0 | (0-0.145) | 0.007 | (0-0.049) | 3.131E−01 | 1.000E+00 |
| Cholesterol glucuronide | 0.48 | (0.4-0.56) | 0 | (0-0.02) | 0.022 | (0-0.064) | 2.994E−01 | 1.000E+00 |
| Lysophosphatidylcholine(20:1) | 0.48 | (0.4-0.55) | 0 | (0-0.015) | 0.015 | (0-0.049) | 2.742E−01 | 1.000E+00 |
| D-RIBOSE 5-PHOSPHATE | 0.48 | (0.4-0.55) | 0 | (0-0.025) | 0.015 | (0-0.052) | 2.913E−01 | 1.000E+00 |
| methionine sulfoxide | 0.48 | (0.4-0.55) | 0.01 | (0-0.06) | 0 | (0-0.064) | 2.873E−01 | 1.000E+00 |
| PHOSPHOCHOLINE | 0.48 | (0.4-0.55) | 0 | (0-0.035) | 0.052 | (0.015-0.097) | 2.794E−01 | 1.000E+00 |
| sn-glycero-3-Phosphoethanolamine | 0.48 | (0.4-0.55) | 0 | (0-0.04) | 0.052 | (0.004-0.104) | 2.880E−01 | 1.000E+00 |
| Prostaglandin D2 | 0.48 | (0.4-0.55) | 0 | (0-0.015) | 0 | (0-0.022) | 2.664E−01 | 1.000E+00 |
| THYMINE | 0.48 | (0.4-0.55) | 0 | (0-0.02) | 0.06 | (0.022-0.116) | 2.581E−01 | 1.000E+00 |
| THIOPURINE S-METHYLETHER | 0.48 | (0.4-0.55) | 0 | (0-0.03) | 0.037 | (0-0.09) | 2.722E−01 | 1.000E+00 |
| THIOUREA | 0.48 | (0.4-0.55) | 0.02 | (0-0.065) | 0 | (0-0.052) | 2.761E−01 | 1.000E+00 |
| Oleamide | 0.48 | (0.4-0.55) | 0.02 | (0-0.06) | 0.022 | (0-0.06) | 2.588E−01 | 1.000E+00 |
| Free Fatty Acid (20:4) (arachidonic acid) | 0.48 | (0.4-0.55) | 0.02 | (0-0.085) | 0 | (0-0.049) | 2.794E−01 | 1.000E+00 |
| PTERIN | 0.48 | (0.4-0.55) | 0 | (0-0.03) | 0 | (0-0.037) | 2.913E−01 | 1.000E+00 |
| N-ACETYLPUTRESCINE | 0.48 | (0.4-0.55) | 0.05 | (0.01-0.115) | 0.022 | (0-0.071) | 2.967E−01 | 1.000E+00 |
| simvastatin hydroxy acid | 0.48 | (0.4-0.55) | 0.01 | (0-0.065) | 0.007 | (0-0.057) | 2.664E−01 | 1.000E+00 |
| L-VALINE | 0.48 | (0.4-0.55) | 0 | (0-0.015) | 0.022 | (0-0.071) | 2.594E−01 | 1.000E+00 |
| Pentanoic Acid | 0.48 | (0.4-0.55) | 0.02 | (0-0.07) | 0.022 | (0.004-0.049) | 2.619E−01 | 1.000E+00 |
| CUDA_istd | 0.48 | (0.4-0.55) | 0 | (0-0.03) | 0 | (0-0.026) | 2.683E−01 | 1.000E+00 |
| Aspartyl-Threonine | 0.47 | (0.4-0.55) | 0.01 | (0-0.045) | 0.015 | (0-0.06) | 2.494E−01 | 1.000E+00 |
| N-AMIDINO-L-ASPARTATE | 0.47 | (0.4-0.55) | 0 | (0-0.02) | 0.007 | (0-0.097) | 2.342E−01 | 1.000E+00 |
| 4-GUANIDINOBUTANOATE | 0.47 | (0.4-0.55) | 0.01 | (0-0.035) | 0.007 | (0-0.034) | 2.494E−01 | 1.000E+00 |
| HEPTANOIC ACID | 0.47 | (0.4-0.55) | 0 | (0-0.045) | 0.007 | (0-0.06) | 2.366E−01 | 1.000E+00 |
| 4-IMIDAZOLEACETIC ACID | 0.47 | (0.4-0.55) | 0.02 | (0-0.065) | 0.007 | (0-0.03) | 2.463E−01 | 1.000E+00 |
| L-METHIONINE | 0.47 | (0.4-0.55) | 0 | (0-0.03) | 0 | (0-0.022) | 2.366E−01 | 1.000E+00 |
| 3-HYDROXYBUTANOIC ACID | 0.47 | (0.4-0.55) | 0 | (0-0.055) | 0.007 | (0-0.045) | 2.288E−01 | 1.000E+00 |
| D-GLUCOSAMINE 6-PHOSPHATE | 0.47 | (0.4-0.55) | 0.01 | (0-0.03) | 0.007 | (0-0.034) | 2.402E−01 | 1.000E+00 |
| Ecgonine | 0.47 | (0.4-0.55) | 0 | (0-0.06) | 0.007 | (0-0.06) | 2.402E−01 | 1.000E+00 |
| Propionic Acid | 0.47 | (0.4-0.55) | 0 | (0-0.005) | 0.03 | (0.007-0.082) | 2.342E−01 | 1.000E+00 |
| Decanoic Acid | 0.47 | (0.4-0.55) | 0 | (0-0.03) | 0.03 | (0-0.075) | 2.348E−01 | 1.000E+00 |
| Free Fatty Acid (18:2) (linoleic acid) | 0.47 | (0.4-0.55) | 0 | (0-0.06) | 0.022 | (0.007-0.086) | 2.544E−01 | 1.000E+00 |
| Putative Glycodeoxycholic acid (GDCA) | 0.47 | (0.4-0.55) | 0 | (0-0.02) | 0.015 | (0-0.127) | 2.276E−01 | 1.000E+00 |
| Acylcarnitine(C4:0) | 0.47 | (0.4-0.55) | 0 | (0-0.02) | 0.022 | (0-0.056) | 2.212E−01 | 1.000E+00 |
| Glycodeoxycholic acid (GDCA) | 0.47 | (0.4-0.54) | 0 | (0-0.025) | 0.045 | (0-0.09) | 2.189E−01 | 1.000E+00 |
| L-HOMOCYSTEINE THIOLACTONE | 0.47 | (0.39-0.55) | 0.02 | (0-0.06) | 0.022 | (0-0.049) | 2.183E−01 | 1.000E+00 |
| HOMOCYSTEINE | 0.47 | (0.39-0.55) | 0.03 | (0-0.06) | 0.007 | (0-0.041) | 2.149E−01 | 1.000E+00 |
| Hydroxyphenyllactic acid | 0.47 | (0.39-0.54) | 0.03 | (0-0.07) | 0.007 | (0-0.026) | 1.880E−01 | 1.000E+00 |
| NEPSILON.NEPSILON.NEPSILON- | 0.47 | (0.39-0.54) | 0.01 | (0-0.04) | 0.015 | (0-0.045) | 1.870E−01 | 1.000E+00 |
| TRIMETHYLLYSINE | | | | | | | | |
| URIDINE 5′-DIPHOSPHOGLUCOSE | 0.47 | (0.39-0.54) | 0.015 | (0-0.06) | 0.022 | (0-0.056) | 1.833E−01 | 1.000E+00 |
| CORTISONE | 0.47 | (0.39-0.54) | 0 | (0-0.02) | 0.015 | (0-0.045) | 2.103E−01 | 1.000E+00 |
| HIPPURATE | 0.47 | (0.39-0.54) | 0.01 | (0-0.045) | 0.007 | (0-0.03) | 1.812E−01 | 1.000E+00 |
| CAFFEINE | 0.47 | (0.39-0.54) | 0 | (0-0.03) | 0.007 | (0-0.049) | 1.880E−01 | 1.000E+00 |
| NORLEUCINE | 0.47 | (0.39-0.54) | 0.03 | (0-0.07) | 0.022 | (0.004-0.079) | 1.864E−01 | 1.000E+00 |
| 5-AMINOIMIDAZOLE-4- | 0.47 | (0.39-0.54) | 0.03 | (0-0.065) | 0 | (0-0.026) | 1.976E−01 | 1.000E+00 |
| CARBOXAMIDE-1-BETA-D- | | | | | | | | |
| RIBOFURANOSYL 5′- | | | | | | | | |
| MONOPHOSPHATE | | | | | | | | |
| Hydroxypropionylcarnitine | 0.47 | (0.39-0.54) | 0.01 | (0-0.04) | 0.015 | (0-0.037) | 2.009E−01 | 1.000E+00 |
| UROCANATE | 0.47 | (0.39-0.54) | 0.02 | (0-0.065) | 0.03 | (0.004-0.079) | 1.875E−01 | 1.000E+00 |
| Indole-3-lactic acid | 0.47 | (0.39-0.54) | 0 | (0-0.03) | 0.03 | (0-0.079) | 1.818E−01 | 1.000E+00 |
| Free Fatty Acid (20:5) (eicosapentaenoic | 0.47 | (0.39-0.54) | 0 | (0-0.05) | 0.026 | (0-0.079) | 2.109E−01 | 1.000E+00 |
| acid) | | | | | | | | |
| DIMETHYLBENZIMIDAZOLE | 0.47 | (0.39-0.54) | 0.01 | (0-0.04) | 0.03 | (0.007-0.067) | 2.042E−01 | 1.000E+00 |
| cADP-RIBOSE | 0.47 | (0.39-0.54) | 0 | (0-0.03) | 0.007 | (0-0.064) | 1.880E−01 | 1.000E+00 |
| Eicosapentaenoic Acid (n-3) | 0.47 | (0.39-0.54) | 0.01 | (0-0.035) | 0.03 | (0.007-0.067) | 1.971E−01 | 1.000E+00 |
| 4-HYDROXYPHENYLACETATE | 0.47 | (0.39-0.54) | 0.01 | (0-0.035) | 0.007 | (0-0.037) | 1.993E−01 | 1.000E+00 |
| 3-AMINO-5-HYDROXYBENZOIC ACID | 0.47 | (0.39-0.54) | 0 | (0-0.08) | 0.007 | (0-0.052) | 2.109E−01 | 1.000E+00 |
| D-GLUCONATE | 0.46 | (0.39-0.54) | 0.03 | (0.01-0.085) | 0.019 | (0-0.049) | 1.677E−01 | 1.000E+00 |
| D-ASPARTATE | 0.46 | (0.39-0.54) | 0 | (0-0.03) | 0.007 | (0-0.03) | 1.619E−01 | 1.000E+00 |
| Thromboxane B2 | 0.46 | (0.39-0.54) | 0 | (0-0.04) | 0 | (0-0.037) | 1.562E−01 | 1.000E+00 |
| MELIBIOSE | 0.46 | (0.39-0.54) | 0.02 | (0-0.05) | 0 | (0-0.12) | 1.761E−01 | 1.000E+00 |
| 4-HYDROXYBENZALDEHYDE | 0.46 | (0.39-0.54) | 0.01 | (0-0.03) | 0.022 | (0.004-0.064) | 1.761E−01 | 1.000E+00 |
| ALPHA-D-GALACTOSE 1-PHOSPHATE | 0.46 | (0.39-0.54) | 0.04 | (0.01-0.11) | 0.022 | (0-0.067) | 1.761E−01 | 1.000E+00 |
| 3′-CMP | 0.46 | (0.39-0.54) | 0 | (0-0.04) | 0.037 | (0-0.086) | 1.751E−01 | 1.000E+00 |
| RETINOATE | 0.46 | (0.39-0.54) | 0.01 | (0-0.065) | 0.022 | (0.007-0.075) | 1.731E−01 | 1.000E+00 |
| 1-(2,3-Dihydro-1H-pyrrolizin-5-yl)-1,4- | 0.46 | (0.39-0.54) | 0.03 | (0-0.09) | 0.03 | (0.004-0.056) | 1.534E−01 | 1.000E+00 |
| pentanedione | | | | | | | | |
| CYTIDINE 5′-TRIPHOSPHATE | 0.46 | (0.39-0.54) | 0.01 | (0-0.06) | 0.022 | (0-0.06) | 1.576E−01 | 1.000E+00 |
| 3-METHYL-2-OXINDOLE | 0.46 | (0.39-0.54) | 0.03 | (0.01-0.085) | 0.007 | (0-0.056) | 1.802E−01 | 1.000E+00 |
| DEOXYCORTICOSTERONE ACETATE | 0.46 | (0.39-0.54) | 0.02 | (0-0.06) | 0.03 | (0.007-0.075) | 1.520E−01 | 1.000E+00 |
| L-2-PHOSPHOGLYCERIC ACID | 0.46 | (0.39-0.54) | 0.01 | (0-0.05) | 0.007 | (0-0.026) | 1.777E−01 | 1.000E+00 |
| 6-PHOSPHOGLUCONIC ACID | 0.46 | (0.39-0.54) | 0.005 | (0-0.03) | 0.007 | (0-0.06) | 1.633E−01 | 1.000E+00 |
| CITRAMALATE | 0.46 | (0.39-0.54) | 0 | (0-0.06) | 0.022 | (0-0.06) | 1.576E−01 | 1.000E+00 |
| HYDROQUINONE | 0.46 | (0.39-0.53) | 0 | (0-0.01) | 0.022 | (0-0.067) | 1.421E−01 | 1.000E+00 |
| Alloxan [M + NH4]+; TRIGONELLINE | 0.46 | (0.39-0.53) | 0 | (0-0.015) | 0.045 | (0.007-0.082) | 1.475E−01 | 1.000E+00 |
| GLYCOCHOLATE | 0.46 | (0.39-0.53) | 0.01 | (0-0.04) | 0.015 | (0-0.056) | 1.516E−01 | 1.000E+00 |
| THYMIDINE 5′-MONOPHOSPHATE | 0.46 | (0.38-0.54) | 0.01 | (0-0.04) | 0.007 | (0-0.045) | 1.493E−01 | 1.000E+00 |
| Glycyl-threonine | 0.46 | (0.38-0.53) | 0.01 | (0-0.06) | 0.019 | (0-0.075) | 1.290E−01 | 1.000E+00 |
| CAFFEATE | 0.46 | (0.38-0.53) | 0 | (0-0) | 0.007 | (0-0.071) | 1.365E−01 | 1.000E+00 |
| 5,6-dihydrouridine | 0.46 | (0.38-0.53) | 0 | (0-0.03) | 0.022 | (0-0.052) | 1.461E−01 | 1.000E+00 |
| INDOLE-3-ACETAMIDE | 0.46 | (0.38-0.53) | 0.03 | (0.005-0.07) | 0 | (0-0.022) | 1.277E−01 | 1.000E+00 |
| Metformin | 0.46 | (0.38-0.53) | 0 | (0-0.05) | 0.037 | (0.007-0.086) | 1.282E−01 | 1.000E+00 |
| DIETHYL 2-METHYL-3- | 0.46 | (0.38-0.53) | 0 | (0-0.015) | 0.034 | (0-0.105) | 1.361E−01 | 1.000E+00 |
| OXOSUCCINATE | | | | | | | | |
| INDOLE | 0.46 | (0.38-0.53) | 0.01 | (0-0.03) | 0.007 | (0-0.045) | 1.237E−01 | 1.000E+00 |
| MALEIC ACID | 0.46 | (0.38-0.53) | 0.03 | (0-0.09) | 0.045 | (0.007-0.108) | 1.421E−01 | 1.000E+00 |
| L-GLUTAMIC ACID_In Source | 0.46 | (0.38-0.53) | 0 | (0-0.01) | 0.03 | (0.007-0.079) | 1.298E−01 | 1.000E+00 |
| N-ACETYL-DL-SERINE_breakthrough | 0.46 | (0.38-0.53) | 0 | (0-0.03) | 0.015 | (0-0.041) | 1.290E−01 | 1.000E+00 |
| Sphingomyelin(32:1) | 0.46 | (0.38-0.53) | 0 | (0-0.03) | 0.075 | (0.018-0.146) | 1.213E−01 | 1.000E+00 |
| D-GLYCERIC ACID | 0.45 | (0.38-0.53) | 0 | (0-0.045) | 0.015 | (0-0.037) | 1.092E−01 | 1.000E+00 |
| S-(5′-ADENOSYL)-L-HOMOCYSTEINE | 0.45 | (0.38-0.53) | 0.01 | (0-0.04) | 0.015 | (0-0.071) | 1.171E−01 | 1.000E+00 |
| 2-QUINOLINECARBOXYLIC ACID | 0.45 | (0.38-0.53) | 0 | (0-0.035) | 0.037 | (0.007-0.082) | 1.096E−01 | 1.000E+00 |
| HOMOSERINE | 0.45 | (0.38-0.53) | 0.02 | (0-0.13) | 0.015 | (0-0.045) | 1.099E−01 | 1.000E+00 |
| N-ACETYLGLYCINE | 0.45 | (0.38-0.53) | 0 | (0-0.04) | 0.007 | (0-0.052) | 1.056E−01 | 1.000E+00 |
| INDOXYL SULFATE | 0.45 | (0.38-0.53) | 0 | (0-0.03) | 0.007 | (0-0.045) | 1.190E−01 | 1.000E+00 |
| Nicardipine | 0.45 | (0.38-0.53) | 0 | (0-0.025) | 0.022 | (0-0.104) | 9.863E−02 | 1.000E+00 |
| Phenacetin | 0.45 | (0.38-0.53) | 0 | (0-0.04) | 0.015 | (0-0.06) | 1.133E−01 | 1.000E+00 |
| MELATONIN | 0.45 | (0.38-0.53) | 0 | (0-0.025) | 0.03 | (0.004-0.067) | 1.035E−01 | 1.000E+00 |
| 3-METHOXY-L-TYROSINE | 0.45 | (0.38-0.53) | 0 | (0-0.07) | 0.015 | (0-0.037) | 1.035E−01 | 1.000E+00 |
| PYRIXODINE_Breakthrough | 0.45 | (0.38-0.53) | 0 | (0-0.02) | 0.03 | (0-0.067) | 1.155E−01 | 1.000E+00 |
| L-CYSTEIC ACID | 0.45 | (0.38-0.53) | 0.03 | (0-0.07) | 0.015 | (0-0.045) | 1.063E−01 | 1.000E+00 |
| URIDINE 5′-DIPHOSPHATE | 0.45 | (0.38-0.53) | 0 | (0-0.04) | 0.03 | (0-0.067) | 1.171E−01 | 1.000E+00 |
| Free Fatty Acid (22:6) | 0.45 | (0.38-0.53) | 0.005 | (0-0.04) | 0.022 | (0-0.075) | 1.035E−01 | 1.000E+00 |
| (docosahexaenoic acid) | | | | | | | | |
| D-GLUCOSE-6-PHOSPHATE | 0.45 | (0.38-0.53) | 0 | (0-0.03) | 0.015 | (0-0.049) | 1.056E−01 | 1.000E+00 |
| PHOSPHOCHOLINE_insource | 0.45 | (0.38-0.53) | 0 | (0-0.04) | 0.03 | (0.004-0.075) | 1.140E−01 | 1.000E+00 |
| L-ARGININE | 0.45 | (0.38-0.52) | 0 | (0-0) | 0.007 | (0-0.057) | 9.397E−02 | 1.000E+00 |
| ELAIDIC ACID | 0.45 | (0.38-0.52) | 0 | (0-0.01) | 0.022 | (0-0.064) | 9.628E−02 | 1.000E+00 |
| L-PROLINE | 0.45 | (0.37-0.53) | 0.02 | (0-0.06) | 0.015 | (0-0.056) | 9.695E−02 | 1.000E+00 |
| METHYLMALONATE | 0.45 | (0.37-0.53) | 0.015 | (0-0.05) | 0.015 | (0-0.045) | 9.695E−02 | 1.000E+00 |
| hydroxyisovaleroyl carnitine | 0.45 | (0.37-0.53) | 0.02 | (0-0.08) | 0.015 | (0-0.075) | 9.729E−02 | 1.000E+00 |
| Lysophosphatidylethanolamine(16:0) | 0.45 | (0.37-0.52) | 0.04 | (0-0.07) | 0.015 | (0-0.052) | 8.243E−02 | 1.000E+00 |
| HOMOGENTISATE | 0.45 | (0.37-0.52) | 0 | (0-0.025) | 0.034 | (0-0.079) | 8.979E−02 | 1.000E+00 |
| 5,6-dihydrouridine [M + K]+ | 0.45 | (0.37-0.52) | 0.04 | (0.005-0.085) | 0.022 | (0-0.06) | 8.759E−02 | 1.000E+00 |
| INOSINE 5′-PHOSPHATE_breakthrough | 0.45 | (0.37-0.52) | 0 | (0-0) | 0.015 | (0-0.045) | 7.553E−02 | 1.000E+00 |
| LAUROYLCARNITINE | 0.45 | (0.37-0.52) | 0 | (0-0.02) | 0.03 | (0-0.075) | 8.697E−02 | 1.000E+00 |
| Indole-derivative | 0.45 | (0.37-0.52) | 0 | (0-0.03) | 0.022 | (0-0.075) | 8.947E−02 | 1.000E+00 |
| INDOLE-3-ACETATE | 0.45 | (0.37-0.52) | 0.02 | (0-0.06) | 0.03 | (0.007-0.064) | 8.947E−02 | 1.000E+00 |
| L-METHIONINE SULFOXIMINE | 0.45 | (0.37-0.52) | 0.025 | (0-0.07) | 0 | (0-0.052) | 7.778E−02 | 1.000E+00 |
| Lysophosphatidylethanolamine(22:6) | 0.44 | (0.37-0.52) | 0 | (0-0.03) | 0 | (0-0.019) | 7.066E−02 | 1.000E+00 |
| LEUKOTRIENE B4 | 0.44 | (0.37-0.52) | 0 | (0-0.05) | 0.015 | (0-0.075) | 7.172E−02 | 1.000E+00 |
| 12(s)-HETE | 0.44 | (0.37-0.52) | 0.02 | (0-0.07) | 0.007 | (0-0.049) | 7.415E−02 | 1.000E+00 |
| INOSINE 5′-DIPHOSPHATE | 0.44 | (0.37-0.52) | 0.01 | (0-0.07) | 0.011 | (0-0.06) | 6.429E−02 | 1.000E+00 |
| CITRATE | 0.44 | (0.37-0.52) | 0.01 | (0-0.055) | 0.019 | (0-0.056) | 7.066E−02 | 1.000E+00 |
| SUBERIC ACID | 0.44 | (0.37-0.52) | 0.02 | (0-0.06) | 0 | (0-0.034) | 6.503E−02 | 1.000E+00 |
| 3.4-DIHYDROXYPHENYLACETATE | 0.44 | (0.37-0.52) | 0 | (0-0) | 0.015 | (0-0.071) | 6.628E−02 | 1.000E+00 |
| FERULATE | 0.44 | (0.37-0.52) | 0 | (0-0.015) | 0.022 | (0.004-0.052) | 7.199E−02 | 1.000E+00 |
| 2-HYDROXYPYRIDINE | 0.44 | (0.37-0.52) | 0 | (0-0.03) | 0 | (0-0.056) | 6.211E−02 | 1.000E+00 |
| QUINATE | 0.44 | (0.37-0.52) | 0 | (0-0) | 0.037 | (0.007-0.086) | 6.454E−02 | 1.000E+00 |
| D-GLUCURONIC ACID | 0.44 | (0.37-0.52) | 0 | (0-0) | 0.015 | (0-0.101) | 6.729E−02 | 1.000E+00 |
| Indoleacrylic acid | 0.44 | (0.37-0.52) | 0.01 | (0-0.03) | 0.03 | (0-0.093) | 7.334E−02 | 1.000E+00 |
| CHENODEOXYCHOLATE | 0.44 | (0.37-0.52) | 0 | (0-0.04) | 0.007 | (0-0.037) | 7.092E−02 | 1.000E+00 |
| 3′.5′-CYCLIC AMP | 0.44 | (0.37-0.52) | 0.02 | (0-0.055) | 0 | (0-0.034) | 6.429E−02 | 1.000E+00 |
| NICOTINAMIDE | 0.44 | (0.37-0.52) | 0 | (0-0.02) | 0 | (0-0.041) | 7.172E−02 | 1.000E+00 |
| Ceramide(40:1) | 0.44 | (0.37-0.52) | 0 | (0-0.025) | 0.03 | (0-0.064) | 7.199E−02 | 1.000E+00 |
| Acylcarnitine(C10:0) | 0.44 | (0.37-0.52) | 0 | (0-0.045) | 0.015 | (0-0.064) | 7.039E−02 | 1.000E+00 |
| O-ACETYL-L-SERINE | 0.44 | (0.37-0.51) | 0.02 | (0-0.04) | 0.015 | (0-0.056) | 5.884E−02 | 1.000E+00 |
| 2′.4′-DIHYDROXYACETOPHENONE | 0.44 | (0.37-0.51) | 0.01 | (0-0.05) | 0.007 | (0-0.041) | 5.770E−02 | 1.000E+00 |
| N-ACETYL-DL-GLUTAMIC ACID | 0.44 | (0.37-0.51) | 0 | (0-0.06) | 0.022 | (0-0.086) | 5.439E−02 | 1.000E+00 |
| D-GLUCONO-1.5-LACTONE | 0.44 | (0.37-0.51) | 0.01 | (0-0.03) | 0.007 | (0-0.045) | 5.953E−02 | 1.000E+00 |
| cis-9, cis-12-Octadecadienoic acid | 0.44 | (0.36-0.51) | 0.04 | (0.005-0.085) | 0.015 | (0-0.037) | 4.982E−02 | 1.000E+00 |
| CORTICOSTERONE | 0.44 | (0.36-0.51) | 0.01 | (0-0.03) | 0 | (0-0.022) | 4.823E−02 | 1.000E+00 |
| 5-OXO-L-PROLINE | 0.44 | (0.36-0.51) | 0.01 | (0-0.04) | 0.045 | (0.007-0.097) | 5.592E−02 | 1.000E+00 |
| DEOXYCHOLATE | 0.44 | (0.36-0.51) | 0.01 | (0-0.03) | 0.015 | (0-0.041) | 4.481E−02 | 1.000E+00 |
| 2-AMINOPHENOL | 0.44 | (0.36-0.51) | 0.02 | (0-0.055) | 0 | (0-0.049) | 4.863E−02 | 1.000E+00 |
| TRIGONELLINE | 0.44 | (0.36-0.51) | 0 | (0-0.035) | 0.007 | (0-0.03) | 4.574E−02 | 1.000E+00 |
| TRIGONELLINE_split peak | 0.44 | (0.36-0.51) | 0 | (0-0.03) | 0.007 | (0-0.045) | 4.863E−02 | 1.000E+00 |
| URIDINE 5′-DIPHOSPHOGALACTOSE | 0.44 | (0.36-0.51) | 0.01 | (0-0.04) | 0.022 | (0-0.064) | 4.863E−02 | 1.000E+00 |
| DEOXYCARNITINE | 0.44 | (0.36-0.51) | 0.01 | (0-0.055) | 0.022 | (0.007-0.06) | 4.784E−02 | 1.000E+00 |
| CHOLATE | 0.44 | (0.36-0.51) | 0 | (0-0.03) | 0.007 | (0-0.071) | 4.843E−02 | 1.000E+00 |
| ALPHA-D-GLUCOSE 1-PHOSPHATE | 0.44 | (0.36-0.51) | 0.01 | (0-0.035) | 0.015 | (0-0.052) | 5.312E−02 | 1.000E+00 |
| Myristelaidic Acid | 0.44 | (0.36-0.51) | 0 | (0-0.05) | 0.022 | (0-0.097) | 4.726E−02 | 1.000E+00 |
| CARNOSINE | 0.43 | (0.36-0.51) | 0 | (0-0.03) | 0.022 | (0-0.049) | 3.955E−02 | 1.000E+00 |
| Benzoylecgonine | 0.43 | (0.36-0.51) | 0 | (0-0.03) | 0 | (0-0.052) | 3.556E−02 | 1.000E+00 |
| Free Fatty Acid (16:1) (palmitoleic acid) | 0.43 | (0.36-0.51) | 0 | (0-0.03) | 0.007 | (0-0.06) | 4.177E−02 | 1.000E+00 |
| Methylpyrazine | 0.43 | (0.36-0.51) | 0 | (0-0.03) | 0 | (0-0.026) | 3.541E−02 | 1.000E+00 |
| (R)-MALATE | 0.43 | (0.36-0.51) | 0.02 | (0-0.06) | 0.015 | (0-0.052) | 3.541E−02 | 1.000E+00 |
| 1-HYDROXY-2-NAPHTHOATE | 0.43 | (0.36-0.5) | 0 | (0-0.02) | 0.007 | (0-0.056) | 3.421E−02 | 1.000E+00 |
| Free Fatty Acid (18:3) (linolenic acid) | 0.43 | (0.36-0.5) | 0 | (0-0.05) | 0.022 | (0-0.067) | 3.218E−02 | 1.000E+00 |
| TRANS-ACONITATE | 0.43 | (0.36-0.5) | 0.01 | (0-0.06) | 0.03 | (0-0.079) | 3.347E−02 | 1.000E+00 |
| (R.R)-TARTARIC ACID | 0.43 | (0.35-0.5) | 0.01 | (0-0.035) | 0.007 | (0-0.06) | 3.094E−02 | 1.000E+00 |
| PIPECOLATE | 0.43 | (0.35-0.5) | 0 | (0-0) | 0.015 | (0-0.037) | 2.818E−02 | 1.000E+00 |
| Choline | 0.43 | (0.35-0.5) | 0 | (0-0.03) | 0.015 | (0-0.041) | 2.587E−02 | 1.000E+00 |
| PHYLLOQUINONE | 0.43 | (0.35-0.5) | 0.02 | (0-0.055) | 0.015 | (0-0.041) | 2.934E−02 | 1.000E+00 |
| niacinamide | 0.43 | (0.35-0.5) | 0.02 | (0-0.06) | 0.015 | (0-0.049) | 2.646E−02 | 1.000E+00 |
| Lysophosphatidylcholine(26:0) | 0.43 | (0.35-0.5) | 0 | (0-0.03) | 0.037 | (0.007-0.086) | 2.973E−02 | 1.000E+00 |
| CREATINE | 0.42 | (0.35-0.5) | 0.02 | (0-0.06) | 0.011 | (0-0.064) | 1.940E−02 | 1.000E+00 |
| ESTRADIOL-17ALPHA | 0.42 | (0.35-0.5) | 0 | (0-0.02) | 0 | (0-0.007) | 2.361E−02 | 1.000E+00 |
| L-LYSINE | 0.42 | (0.35-0.5) | 0 | (0-0.005) | 0.007 | (0-0.056) | 2.112E−02 | 1.000E+00 |
| INOSINE_in source | 0.42 | (0.35-0.5) | 0 | (0-0.04) | 0.015 | (0-0.052) | 2.318E−02 | 1.000E+00 |
| 3-METHYLHISTAMINE_split peak | 0.42 | (0.35-0.5) | 0.02 | (0-0.05) | 0.022 | (0-0.052) | 1.903E−02 | 1.000E+00 |
| Lysophosphatidylcholine(20:5) | 0.42 | (0.35-0.5) | 0 | (0-0.05) | 0.015 | (0-0.071) | 1.885E−02 | 1.000E+00 |
| Prostaglandin A1 | 0.42 | (0.35-0.49) | 0.04 | (0.005-0.085) | 0.03 | (0-0.067) | 1.913E−02 | 1.000E+00 |
| THIAMINE MONOPHOSPHATE | 0.42 | (0.35-0.49) | 0.02 | (0-0.07) | 0.022 | (0-0.052) | 1.958E−02 | 1.000E+00 |
| CATECHOL | 0.42 | (0.35-0.49) | 0 | (0-0.01) | 0.015 | (0-0.068) | 1.885E−02 | 1.000E+00 |
| 3-cis-Hydroxy-b,e-Caroten-3′-one | 0.42 | (0.34-0.49) | 0.01 | (0-0.04) | 0.007 | (0-0.037) | 1.648E−02 | 1.000E+00 |
| Phosphatidylethanolamine(36:4) | 0.42 | (0.34-0.49) | 0 | (0-0.04) | 0.045 | (0.007-0.09) | 1.346E−02 | 1.000E+00 |
| Alpha-N-Phenylacetyl-L-glutamine | 0.42 | (0.34-0.49) | 0 | (0-0.035) | 0 | (0-0.022) | 1.346E−02 | 1.000E+00 |
| Putative_Creatine riboside | 0.42 | (0.34-0.49) | 0 | (0-0) | 0.007 | (0-0.037) | 1.313E−02 | 1.000E+00 |
| Testosterone | 0.42 | (0.34-0.49) | 0 | (0-0.04) | 0.022 | (0.004-0.064) | 1.721E−02 | 1.000E+00 |
| 1-Methylhistidine | 0.42 | (0.34-0.49) | 0.02 | (0-0.07) | 0.015 | (0-0.045) | 1.486E−02 | 1.000E+00 |
| XANTHURENIC ACID | 0.42 | (0.34-0.49) | 0 | (0-0.045) | 0 | (0-0.004) | 1.640E−02 | 1.000E+00 |
| cis-Quinceoxepane [M − H2O + H]+ | 0.42 | (0.34-0.49) | 0.01 | (0-0.06) | 0.007 | (0-0.052) | 1.524E−02 | 1.000E+00 |
| L-NORVALINE | 0.42 | (0.34-0.49) | 0 | (0-0.03) | 0.007 | (0-0.052) | 1.531E−02 | 1.000E+00 |
| HOMOCYSTINE | 0.42 | (0.34-0.49) | 0.02 | (0-0.065) | 0.007 | (0-0.075) | 1.608E−02 | 1.000E+00 |
| 4-HYDROXY-L-PHENYLGLYCINE | 0.42 | (0.34-0.49) | 0 | (0-0.015) | 0 | (0-0.019) | 1.394E−02 | 1.000E+00 |
| URIDINE 5′-TRIPHOSPHATE | 0.42 | (0.34-0.49) | 0 | (0-0.015) | 0.015 | (0-0.086) | 1.373E−02 | 1.000E+00 |
| Acylcarnitine(C8:0) | 0.42 | (0.34-0.49) | 0 | (0-0.03) | 0.015 | (0-0.056) | 1.373E−02 | 1.000E+00 |
| 5-hydroxyindole-3-acetic acid | 0.41 | (0.34-0.49) | 0.01 | (0-0.06) | 0.015 | (0-0.037) | 1.110E−02 | 1.000E+00 |
| NALPHA-ACETYL-L-LYSINE | 0.41 | (0.34-0.49) | 0 | (0-0.06) | 0.007 | (0-0.052) | 1.223E−02 | 1.000E+00 |
| PICOLINIC ACID | 0.41 | (0.34-0.49) | 0.04 | (0.005-0.095) | 0 | (0-0.052) | 1.088E−02 | 1.000E+00 |
| SUCCINATE | 0.41 | (0.34-0.49) | 0 | (0-0.04) | 0.004 | (0-0.03) | 1.012E−02 | 1.000E+00 |
| Taurocholic Acid (TCA) | 0.41 | (0.34-0.49) | 0.01 | (0-0.02) | 0.011 | (0-0.041) | 1.286E−02 | 1.000E+00 |
| Lysophosphatidylcholine(22:6) | 0.41 | (0.34-0.48) | 0 | (0-0.03) | 0.022 | (0.004-0.06) | 8.416E−03 | 1.000E+00 |
| Glycerophosphocholine | 0.41 | (0.34-0.48) | 0 | (0-0.05) | 0.015 | (0-0.052) | 9.207E−03 | 1.000E+00 |
| Sphingomyelin(32:2) | 0.41 | (0.34-0.48) | 0 | (0-0.045) | 0.037 | (0.015-0.093) | 1.033E−02 | 1.000E+00 |
| cis-Quinceoxepane | 0.41 | (0.33-0.48) | 0.01 | (0-0.065) | 0.007 | (0-0.034) | 7.810E−03 | 1.000E+00 |
| LL-2.6-DIAMINOHEPTANEDIOATE | 0.4 | (0.32-0.47) | 0 | (0-0.03) | 0.007 | (0-0.053) | 3.562E−03 | 1.000E+00 |
| ADENINE | 0.4 | (0.32-0.47) | 0.01 | (0-0.04) | 0.015 | (0-0.045) | 4.387E−03 | 1.000E+00 |
| L-carnitine | 0.39 | (0.32-0.47) | 0 | (0-0.035) | 0.015 | (0-0.037) | 2.330E−03 | 1.000E+00 |
| 3-SULFINO-L-ALANINE | 0.39 | (0.32-0.47) | 0.01 | (0-0.035) | 0 | (0-0.015) | 2.206E−03 | 1.000E+00 |
| Dodecanedioylcarnitine | 0.39 | (0.32-0.47) | 0 | (0-0) | 0.015 | (0-0.041) | 2.663E−03 | 1.000E+00 |
| CYSTEINEGLUTATHIONE | 0.39 | (0.32-0.47) | 0 | (0-0) | 0.022 | (0-0.064) | 2.777E−03 | 1.000E+00 |
| DISSULFIDE_IN SOURCE | | | | | | | | |
| FRAGMENT OF GSSG | | | | | | | | |
| Lysophosphatidylcholine(17:1) | 0.39 | (0.32-0.46) | 0.01 | (0-0.06) | 0 | (0-0.022) | 2.139E−03 | 1.000E+00 |
| Chavicol O-beta-glucopyranoside [M + K]+ | 0.39 | (0.32-0.46) | 0 | (0-0) | 0.015 | (0-0.052) | 2.219E−03 | 1.000E+00 |
| 4-HYDROXY-L-PROLINE | 0.39 | (0.32-0.46) | 0.02 | (0-0.04) | 0.03 | (0-0.067) | 2.477E−03 | 1.000E+00 |
| Coumaric Acid | 0.39 | (0.31-0.46) | 0 | (0-0.04) | 0.022 | (0-0.052) | 1.423E−03 | 6.759E−01 |
| Plas_Lysophosphatidylcholine(P-18:0/0:0) | 0.38 | (0.31-0.46) | 0 | (0-0.06) | 0.022 | (0-0.052) | 1.064E−03 | 5.054E−01 |
| or Plas_Lysophosphatidylcholine(O-18:1) | | | | | | | | |
| Ursodeoxycholic acid (UDCA) | 0.38 | (0.31-0.46) | 0 | (0-0) | 0.015 | (0-0.041) | 1.285E−03 | 6.104E−01 |
| GLUTATHIONE DISSULFIDE | 0.38 | (0.31-0.46) | 0 | (0-0.005) | 0 | (0-0.041) | 1.136E−03 | 5.396E−01 |
| 1-linoleoylglycerol | 0.38 | (0.31-0.45) | 0 | (0-0) | 0.007 | (0-0.034) | 9.969E−04 | 4.735E−01 |
| 1-METHYLNICOTINAMIDE | 0.38 | (0.31-0.45) | 0.01 | (0-0.05) | 0.007 | (0-0.026) | 7.339E−04 | 3.486E−01 |
| Hyaluronic acid | 0.38 | (0.31-0.45) | 0.01 | (0-0.03) | 0 | (0-0) | 8.117E−04 | 3.856E−01 |
| Phosphatidylcholine(36:5) | 0.38 | (0.3-0.45) | 0 | (0-0.04) | 0.022 | (0.007-0.052) | 5.785E−04 | 2.748E−01 |
| piperine | 0.37 | (0.3-0.45) | 0 | (0-0.035) | 0.015 | (0-0.041) | 5.326E−04 | 2.530E−01 |
| N(PAI)-METHYL-L-HISTIDINE | 0.37 | (0.3-0.44) | 0.02 | (0-0.04) | 0.007 | (0-0.022) | 3.279E−04 | 1.558E−01 |
| Lysophosphatidylcholine(24:0) | 0.37 | (0.3-0.44) | 0.01 | (0-0.045) | 0.007 | (0-0.045) | 2.510E−04 | 1.192E−01 |
| D-ORNITHINE | 0.37 | (0.3-0.44) | 0 | (0-0) | 0.015 | (0-0.041) | 3.598E−04 | 1.709E−01 |
| PlasLysophosphatidylcholine(P-18:0/0:0) | 0.37 | (0.3-0.44) | 0 | (0-0.04) | 0.015 | (0-0.03) | 3.209E−04 | 1.524E−01 |
| or PlasLysophosphatidylcholine(O-18:1) | | | | | | | | |
| 2.3-DIHYDROXYBENZOATE | 0.37 | (0.3-0.44) | 0 | (0-0) | 0.022 | (0.007-0.06) | 3.009E−04 | 1.429E−01 |
| PARAXANTHINE | 0.37 | (0.3-0.44) | 0 | (0-0) | 0.015 | (0-0.041) | 2.403E−04 | 1.141E−01 |
| GLUTATHIONE | 0.37 | (0.3-0.44) | 0 | (0-0.005) | 0 | (0-0.03) | 4.029E−04 | 1.914E−01 |
| LINOLEATE | 0.37 | (0.29-0.44) | 0.01 | (0-0.06) | 0.015 | (0-0.045) | 2.185E−04 | 1.038E−01 |
| Lysophosphatidylcholine(17:0) | 0.36 | (0.29-0.44) | 0 | (0-0) | 0.007 | (0-0.03) | 1.956E−04 | 9.291E−02 |
| Dodecanoic acid | 0.36 | (0.29-0.44) | 0 | (0-0) | 0 | (0-0.037) | 1.970E−04 | 9.358E−02 |
| N-METHYL-D-ASPARTIC ACID | 0.36 | (0.29-0.43) | 0.01 | (0-0.045) | 0.015 | (0-0.101) | 1.472E−04 | 6.992E−02 |
| HISTAMINE | 0.36 | (0.29-0.43) | 0.015 | (0-0.04) | 0.015 | (0-0.045) | 1.396E−04 | 6.631E−02 |
| PYRIDOXAMINE | 0.36 | (0.29-0.43) | 0 | (0-0.04) | 0.03 | (0.011-0.064) | 9.519E−05 | 4.522E−02 |
| Phosphatidylcholine(31:1) | 0.36 | (0.29-0.43) | 0.01 | (0-0.07) | 0.022 | (0-0.045) | 1.236E−04 | 5.871E−02 |
| L-ASPARAGINE | 0.36 | (0.29-0.43) | 0 | (0-0) | 0.015 | (0-0.037) | 1.094E−04 | 5.197E−02 |
| CREATINE PHOSPHATE | 0.36 | (0.28-0.43) | 0.01 | (0-0.075) | 0.007 | (0-0.056) | 9.086E−05 | 4.316E−02 |
| L-ANSERINE | 0.35 | (0.28-0.43) | 0 | (0-0.02) | 0.015 | (0-0.03) | 7.016E−05 | 3.333E−02 |
| L-N.Gamma.-monomethylarginine | 0.35 | (0.28-0.43) | 0 | (0-0.02) | 0 | (0-0.022) | 7.072E−05 | 3.359E−02 |
| Lysophosphatidylcholine(22:5) | 0.35 | (0.28-0.42) | 0.01 | (0-0.04) | 0.022 | (0-0.06) | 3.371E−05 | 1.601E−02 |
| BENZYL ALCOHOL | 0.35 | (0.28-0.42) | 0 | (0-0.03) | 0.022 | (0-0.06) | 5.143E−05 | 2.443E−02 |
| Lysophosphatidylethanolamine(20:4) | 0.34 | (0.27-0.42) | 0 | (0-0.02) | 0.015 | (0-0.037) | 2.379E−05 | 1.130E−02 |
| Lysophosphatidylcholine(20:4) | 0.34 | (0.27-0.41) | 0 | (0-0.02) | 0.022 | (0.004-0.06) | 1.214E−05 | 5.767E−03 |
| PlasPhosphatidylethanolamine(o-38:5) or | 0.34 | (0.27-0.41) | 0 | (0-0) | 0.015 | (0-0.049) | 1.558E−05 | 7.401E−03 |
| PlasPhosphatidylethanolamine(p-38:4) | | | | | | | | |
| D-SACCHARIC ACID | 0.34 | (0.27-0.41) | 0 | (0-0.04) | 0.007 | (0-0.049) | 2.115E−05 | 1.005E−02 |
| 2-Arachidonylglycerol | 0.34 | (0.27-0.41) | 0 | (0-0.02) | 0.041 | (0-0.075) | 1.038E−05 | 4.931E−03 |
| TMAO | 0.34 | (0.27-0.41) | 0 | (0-0) | 0.007 | (0-0.041) | 1.335E−05 | 6.341E−03 |
| Lysophosphatidylcholine(16:0) | 0.33 | (0.26-0.4) | 0.01 | (0-0.09) | 0.015 | (0-0.03) | 3.515E−06 | 1.670E−03 |
| PlasLysophosphatidylcholine(P-18:0) | 0.33 | (0.26-0.4) | 0.02 | (0-0.075) | 0.007 | (0-0.037) | 3.515E−06 | 1.670E−03 |
| QUINOLINE | 0.33 | (0.26-0.4) | 0 | (0-0.01) | 0.007 | (0-0.037) | 3.177E−06 | 1.509E−03 |
| L-HISTIDINE | 0.33 | (0.26-0.4) | 0 | (0-0.04) | 0.015 | (0-0.037) | 3.547E−06 | 1.685E−03 |
| Ornithine_insource of citrulline | 0.32 | (0.25-0.39) | 0.01 | (0-0.03) | 0.015 | (0-0.037) | 7.809E−07 | 3.709E−04 |
| [M − H2O + H]+ | | | | | | | | |
| 1-(Hydroxymethyl)-5,5-dimethyl-2,4- | 0.32 | (0.25-0.39) | 0.01 | (0-0.03) | 0.007 | (0-0.034) | 9.123E−07 | 4.333E−04 |
| imidazolidinedione_exogenous | | | | | | | | |
| Chavicol O-beta-glucopyranoside | 0.32 | (0.25-0.39) | 0 | (0-0.035) | 0.015 | (0-0.037) | 1.005E−06 | 4.774E−04 |
| Phosphatidylethanolamine(38:3) | 0.31 | (0.25-0.38) | 0 | (0-0) | 0.015 | (0-0.037) | 6.295E−07 | 2.990E−04 |
| Lysophosphatidylcholine(18:0) | 0.31 | (0.24-0.38) | 0 | (0-0.02) | 0.007 | (0-0.03) | 4.452E−07 | 2.115E−04 |
| Lysophosphatidylethanolamine(22:0) | 0.31 | (0.24-0.38) | 0 | (0-0) | 0.007 | (0-0.026) | 2.804E−07 | 1.332E−04 |
| Lysophosphatidic Acid(18:1) | 0.31 | (0.24-0.38) | 0.01 | (0-0.03) | 0.007 | (0-0.022) | 2.108E−07 | 1.001E−04 |
| PlasLysophosphatidylcholine(O-16:0/0:0) | 0.31 | (0.24-0.38) | 0 | (0-0) | 0.007 | (0-0.037) | 3.871E−07 | 1.839E−04 |
| Phosphatidylcholine(36:2) | 0.31 | (0.24-0.38) | 0 | (0-0) | 0.03 | (0-0.075) | 3.832E−07 | 1.820E−04 |
| Acylcarnitine(C18:1) | 0.31 | (0.24-0.37) | 0 | (0-0.01) | 0.022 | (0-0.049) | 1.942E−07 | 9.220E−05 |
| Sphingosine | 0.3 | (0.24-0.37) | 0 | (0-0.01) | 0 | (0-0.022) | 1.470E−07 | 6.980E−05 |
| Lysophosphatidylethanolamine(18:0) | 0.3 | (0.23-0.37) | 0.01 | (0-0.06) | 0.015 | (0-0.037) | 8.902E−08 | 4.230E−05 |
| Lysophosphatidylcholine(16:1) | 0.29 | (0.22-0.36) | 0.01 | (0-0.07) | 0.007 | (0-0.022) | 1.229E−08 | 5.840E−06 |
| Lysophosphatidic Acid(18:0) | 0.29 | (0.22-0.36) | 0 | (0-0.03) | 0.015 | (0-0.041) | 2.167E−08 | 1.030E−05 |
| THEOBROMINE | 0.29 | (0.22-0.35) | 0 | (0-0) | 0 | (0-0.008) | 1.537E−08 | 7.300E−06 |
| Lysophosphatidylcholine(15:0) | 0.28 | (0.22-0.35) | 0 | (0-0) | 0.015 | (0-0.037) | 6.904E−09 | 3.280E−06 |
| Lysophosphatidylethanolamine(19:0) | 0.28 | (0.22-0.35) | 0 | (0-0) | 0.007 | (0-0.049) | 8.096E−09 | 3.850E−06 |
| Ornithine_insource of citrulline | 0.28 | (0.21-0.34) | 0 | (0-0) | 0.022 | (0-0.06) | 2.904E−09 | 1.380E−06 |
| Lysophosphatidylcholine(18:3) | 0.27 | (0.21-0.34) | 0 | (0-0) | 0.011 | (0-0.037) | 1.226E−09 | 5.820E−07 |
| PlasLysophosphatidylcholine(P-16:0) | 0.27 | (0.21-0.34) | 0.01 | (0-0.03) | 0.015 | (0-0.045) | 1.903E−09 | 9.040E−07 |
| L-ORNITHINE | 0.27 | (0.21-0.34) | 0 | (0-0) | 0.015 | (0-0.049) | 1.466E−09 | 6.960E−07 |
| Lysophosphatidylcholine(20:3) | 0.27 | (0.2-0.34) | 0.01 | (0-0.05) | 0.015 | (0-0.045) | 9.527E−10 | 4.530E−07 |
| Lysophosphatidylcholine(18:1) | 0.27 | (0.2-0.33) | 0.01 | (0-0.03) | 0.015 | (0-0.037) | 4.597E−10 | 2.180E−07 |
| Lysophosphatidylethanolamine(18:2) | 0.27 | (0.2-0.33) | 0 | (0-0) | 0.015 | (0-0.034) | 4.654E−10 | 2.210E−07 |
| Lysophosphatidylcholine(14:0) | 0.26 | (0.19-0.32) | 0.01 | (0-0.05) | 0.007 | (0-0.022) | 1.197E−10 | 5.690E−08 |
| Lysophosphatidylethanolamine(18:1) | 0.26 | (0.19-0.32) | 0 | (0-0.03) | 0.015 | (0-0.037) | 9.899E−11 | 4.700E−08 |
| Lysophosphatidylcholine(20:2) | 0.25 | (0.18-0.31) | 0.01 | (0-0.04) | 0.015 | (0-0.037) | 2.586E−11 | 1.230E−08 |
| Lysophosphatidylcholine(18:2) | 0.24 | (0.18-0.31) | 0 | (0-0) | 0.015 | (0-0.037) | 1.284E−11 | 6.100E−09 |
| | Early stage cases (n = 39) versus BPM (n = 134) | | | | | | | |
| Diacetylspermine | 0.73 | (0.64-0.83) | 0.158 | (0-0.317) | 0.063 | (0.03-0.343) | 5.4723E−06 | 2.599E−03 |
| Creatine riboside | 0.68 | (0.58-0.79) | 0 | (0-0.145) | 0 | (0-0.303) | 2.5905E−04 | 1.230E−01 |
| ALPHA-HYDROXYISOBUTYRIC ACID | 0.71 | (0.62-0.8) | 0.026 | (0-0.105) | 0.179 | (0.123-0.265) | 3.8908E−05 | 1.848E−02 |
| Acetylspermidine | 0.59 | (0.48-0.7) | 0 | (0-0.107) | 0.022 | (0.007-0.168) | 4.0266E−02 | 1.000E+00 |
| Diacetylspermidine | 0.66 | (0.56-0.76) | 0.026 | (0-0.132) | 0.06 | (0.018-0.186) | 1.4747E−03 | 7.005E−01 |
| N-ACETYL-L-ASPARTIC ACID | 0.52 | (0.42-0.62) | 0.026 | (0-0.105) | 0.075 | (0.033-0.157) | 3.6779E−01 | 1.000E+00 |
| Monoelaidin | 0.63 | (0.53-0.73) | 0 | (0-0.079) | 0.06 | (0.026-0.15) | 6.9481E−03 | 1.000E+00 |
| N-ALPHA-ACETYL-L-ASPARAGINE | 0.62 | (0.52-0.71) | 0 | (0-0.053) | 0.097 | (0.041-0.239) | 1.4513E−02 | 1.000E+00 |
| 1-METHYLADENOSINE | 0.64 | (0.54-0.74) | 0 | (0-0.079) | 0.041 | (0.007-0.174) | 3.9590E−03 | 1.000E+00 |
| 2-HYDROXYBUTYRIC ACID | 0.6 | (0.49-0.7) | 0.026 | (0-0.079) | 0.03 | (0.007-0.109) | 3.5987E−02 | 1.000E+00 |
| N-ACETYLNEURAMINATE | 0.57 | (0.46-0.68) | 0.026 | (0-0.105) | 0.022 | (0-0.15) | 9.4124E−02 | 1.000E+00 |
| HYPOXANTHINE | 0.57 | (0.47-0.67) | 0 | (0-0.026) | 0.101 | (0.048-0.172) | 1.0310E−01 | 1.000E+00 |
| Heptanoylcarnitine | 0.58 | (0.47-0.69) | 0.053 | (0-0.132) | 0.052 | (0.022-0.131) | 5.9892E−02 | 1.000E+00 |
| INOSINE | 0.59 | (0.49-0.69) | 0 | (0-0.079) | 0.022 | (0-0.198) | 4.5300E−02 | 1.000E+00 |
| L-GLUTAMINE | 0.6 | (0.5-0.71) | 0 | (0-0) | 0.03 | (0-0.093) | 2.9523E−02 | 1.000E+00 |
| DL-5-HYDROXYLYSINE | 0.57 | (0.46-0.68) | 0 | (0-0.079) | 0 | (0-0.09) | 9.5369E−02 | 1.000E+00 |
| 5-OXO-D-PROLINE | 0.52 | (0.4-0.65) | 0 | (0-0.172) | 0.015 | (0-0.071) | 3.4170E−01 | 1.000E+00 |
| Gabapentin | 0.57 | (0.47-0.68) | 0 | (0-0.026) | 0 | (0-0.083) | 8.8669E−02 | 1.000E+00 |
| N-METHYL-L-GLUTAMATE | 0.64 | (0.55-0.74) | 0.026 | (0-0.119) | 0.067 | (0.037-0.292) | 3.7476E−03 | 1.000E+00 |
| DECANOATE | 0.63 | (0.54-0.73) | 0.079 | (0-0.237) | 0.045 | (0.015-0.225) | 5.8232E−03 | 1.000E+00 |
| Sphingomyelin(34:1) | 0.63 | (0.53-0.73) | 0.026 | (0-0.105) | 0.03 | (0.007-0.28) | 7.3887E−03 | 1.000E+00 |
| Prostaglandin F2a | 0.61 | (0.51-0.72) | 0 | (0-0.105) | 0.03 | (0.007-0.131) | 1.5492E−02 | 1.000E+00 |
| D-GLUCURONOLACTONE | 0.62 | (0.52-0.72) | 0 | (0-0.026) | 0.127 | (0.052-0.288) | 1.3208E−02 | 1.000E+00 |
| TYRAMINE | 0.56 | (0.46-0.65) | 0 | (0-0.066) | 0.015 | (0-0.172) | 1.4855E−01 | 1.000E+00 |
| Tyrosyl-Lysine | 0.57 | (0.46-0.67) | 0 | (0-0) | 0.022 | (0-0.153) | 1.0178E−01 | 1.000E+00 |
| N-(3-acetamidopropyl)pyrrolidin-2-one | 0.54 | (0.44-0.65) | 0 | (0-0.093) | 0.052 | (0.022-0.179) | 2.1213E−01 | 1.000E+00 |
| D-(+)-GALACTOSAMINE | 0.61 | (0.51-0.72) | 0.026 | (0-0.132) | 0 | (0-0.135) | 1.6225E−02 | 1.000E+00 |
| Octopamine | 0.65 | (0.54-0.77) | 0.026 | (0-0.158) | 0.03 | (0.007-0.079) | 1.9154E−03 | 9.098E−01 |
| 4-ACETAMIDOBUTANOATE | 0.54 | (0.44-0.64) | 0 | (0-0) | 0.03 | (0.007-0.116) | 2.0682E−01 | 1.000E+00 |
| N-ACETYL-D-MANNOSAMINE | 0.55 | (0.45-0.65) | 0 | (0-0.026) | 0.052 | (0.007-0.179) | 1.5995E−01 | 1.000E+00 |
| DEOXYURIDINE | 0.55 | (0.45-0.66) | 0 | (0-0) | 0.03 | (0.007-0.101) | 1.5287E−01 | 1.000E+00 |
| ADENOSINE | 0.5 | (0.39-0.61) | 0 | (0-0.105) | 0 | (0-0.083) | 4.9190E−01 | 1.000E+00 |
| GLYCEROL 2-PHOSPHATE | 0.57 | (0.47-0.68) | 0 | (0-0.119) | 0.037 | (0.015-0.191) | 8.8078E−02 | 1.000E+00 |
| 3-METHOXYTYRAMINE | 0.53 | (0.42-0.64) | 0 | (0-0.132) | 0 | (0-0.071) | 2.8677E−01 | 1.000E+00 |
| ALPHA-D-GLUCOSE | 0.56 | (0.46-0.66) | 0.053 | (0-0.158) | 0 | (0-0.261) | 1.2666E−01 | 1.000E+00 |
| CITRULLINE | 0.53 | (0.43-0.63) | 0 | (0-0.079) | 0.022 | (0-0.093) | 2.6944E−01 | 1.000E+00 |
| S-HEXYL-GLUTATHIONE | 0.58 | (0.48-0.68) | 0.053 | (0-0.158) | 0.075 | (0.03-0.149) | 6.5344E−02 | 1.000E+00 |
| D-TRYPTOPHAN | 0.54 | (0.44-0.65) | 0.026 | (0-0.132) | 0.011 | (0-0.097) | 2.0471E−01 | 1.000E+00 |
| BETAINE | 0.58 | (0.47-0.68) | 0.026 | (0-0.105) | 0.015 | (0-0.053) | 7.6862E−02 | 1.000E+00 |
| NG, NG-dimethyl-L-arginine | 0.51 | (0.4-0.62) | 0 | (0-0.079) | 0.022 | (0-0.083) | 3.9451E−01 | 1.000E+00 |
| FORMYL-L-METHIONYL PEPTIDE | 0.57 | (0.47-0.68) | 0.026 | (0-0.079) | 0.06 | (0.022-0.168) | 8.5742E−02 | 1.000E+00 |
| DOPAMINE | 0.6 | (0.5-0.69) | 0 | (0-0.105) | 0.082 | (0.033-0.276) | 3.3715E−02 | 1.000E+00 |
| RETINYL PALMITATE | 0.64 | (0.54-0.73) | 0.026 | (0-0.079) | 0.153 | (0.086-0.325) | 5.4091E−03 | 1.000E+00 |
| 2.3-DIPHOSPHO-D-GLYCERIC ACID | 0.56 | (0.45-0.66) | 0.026 | (0-0.093) | 0.015 | (0-0.146) | 1.3935E−01 | 1.000E+00 |
| D-GALACTOSE | 0.48 | (0.37-0.59) | 0.026 | (0-0.105) | 0.022 | (0.007-0.12) | 3.5535E−01 | 1.000E+00 |
| 2.4-DIHYDROXYPYRIMIDINE-5- | 0.56 | (0.46-0.66) | 0.026 | (0-0.132) | 0.045 | (0.007-0.105) | 1.2137E−01 | 1.000E+00 |
| CARBOXYLIC ACID | | | | | | | | |
| Acylcarnitine(C14:0) | 0.56 | (0.46-0.65) | 0.026 | (0-0.093) | 0.112 | (0.048-0.209) | 1.4516E−01 | 1.000E+00 |
| METHYL VANILLATE | 0.6 | (0.5-0.7) | 0 | (0-0.079) | 0.112 | (0.06-0.202) | 3.1561E−02 | 1.000E+00 |
| L-CYSTINE | 0.57 | (0.46-0.68) | 0.026 | (0-0.119) | 0 | (0-0.06) | 1.0713E−01 | 1.000E+00 |
| L-CYSTEINE_In Source | 0.53 | (0.42-0.64) | 0.026 | (0-0.132) | 0.007 | (0-0.026) | 2.9055E−01 | 1.000E+00 |
| N6-(DELTA2-ISOPENTENYL)- | 0.54 | (0.43-0.64) | 0 | (0-0.105) | 0.037 | (0.015-0.127) | 2.4100E−01 | 1.000E+00 |
| ADENINE | | | | | | | | |
| 6-CARBOXYHEXANOATE | 0.57 | (0.47-0.68) | 0.026 | (0-0.079) | 0.104 | (0.048-0.172) | 8.1210E−02 | 1.000E+00 |
| D-PANTOTHENIC ACID_peak 2 | 0.52 | (0.41-0.63) | 0 | (0-0.105) | 0 | (0-0.038) | 3.5810E−01 | 1.000E+00 |
| N-acetyllactosamine | 0.54 | (0.43-0.65) | 0 | (0-0) | 0.052 | (0.022-0.179) | 2.2741E−01 | 1.000E+00 |
| 3-(2- | 0.56 | (0.46-0.65) | 0 | (0-0) | 0.09 | (0.06-0.146) | 1.4770E−01 | 1.000E+00 |
| HYDROXYPHENYL)PROPANOATE | | | | | | | | |
| 3-METHYLHISTAMINE | 0.57 | (0.46-0.68) | 0.105 | (0-0.263) | 0.052 | (0.015-0.112) | 8.8078E−02 | 1.000E+00 |
| Benzofuran | 0.56 | (0.45-0.67) | 0 | (0-0.014) | 0.015 | (0-0.124) | 1.3370E−01 | 1.000E+00 |
| 2-Methylbutyroylcarnitine | 0.54 | (0.43-0.65) | 0.053 | (0-0.158) | 0 | (0-0.091) | 2.2741E−01 | 1.000E+00 |
| SEROTONIN | 0.5 | (0.39-0.6) | 0 | (0-0) | 0.045 | (0.007-0.131) | 4.7278E−01 | 1.000E+00 |
| 3-HYDROXYANTHRANILATE | 0.56 | (0.46-0.66) | 0 | (0-0.079) | 0.104 | (0.052-0.198) | 1.2362E−01 | 1.000E+00 |
| S-(5′-ADENOSYL)-L-METHIONINE | 0.53 | (0.43-0.63) | 0.026 | (0-0.105) | 0.007 | (0-0.106) | 2.9563E−01 | 1.000E+00 |
| 2-AMINO-2-METHYLPROPANOATE | 0.51 | (0.4-0.61) | 0 | (0-0.079) | 0.015 | (0-0.102) | 4.5372E−01 | 1.000E+00 |
| 2-DEOXY-D-GLUCOSE | 0.48 | (0.37-0.58) | 0 | (0-0) | 0.034 | (0.007-0.09) | 3.2426E−01 | 1.000E+00 |
| PHENOL | 0.54 | (0.44-0.64) | 0 | (0-0.026) | 0.09 | (0.045-0.217) | 2.1643E−01 | 1.000E+00 |
| BIOTIN | 0.52 | (0.41-0.64) | 0.132 | (0.053-0.291) | 0.022 | (0-0.101) | 3.2426E−01 | 1.000E+00 |
| PHENETHYLAMINE | 0.53 | (0.42-0.64) | 0 | (0-0.093) | 0.03 | (0.007-0.071) | 2.7310E−01 | 1.000E+00 |
| SPHINGANINE | 0.51 | (0.41-0.61) | 0 | (0-0) | 0.082 | (0.037-0.142) | 4.3768E−01 | 1.000E+00 |
| GALACTITOL | 0.58 | (0.46-0.69) | 0 | (0-0.079) | 0.022 | (0-0.071) | 6.9188E−02 | 1.000E+00 |
| MALTOSE | 0.51 | (0.41-0.62) | 0 | (0-0.079) | 0.022 | (0-0.128) | 3.9593E−01 | 1.000E+00 |
| Trazodone | 0.62 | (0.51-0.74) | 0.105 | (0-0.251) | 0 | (0-0.101) | 9.7862E−03 | 1.000E+00 |
| DETHIOBIOTIN | 0.49 | (0.39-0.58) | 0 | (0-0) | 0.037 | (0-0.146) | 4.0878E−01 | 1.000E+00 |
| 5(s)-HETE | 0.56 | (0.46-0.66) | 0 | (0-0) | 0.03 | (0-0.179) | 1.1338E−01 | 1.000E+00 |
| 4-PYRIDOXATE | 0.48 | (0.38-0.59) | 0 | (0-0) | 0.052 | (0.015-0.161) | 3.7897E−01 | 1.000E+00 |
| BENZALDEHYDE | 0.54 | (0.44-0.65) | 0 | (0-0.093) | 0 | (0-0.191) | 2.0159E−01 | 1.000E+00 |
| Monoethylhexyl phthalic acid | 0.49 | (0.38-0.59) | 0 | (0-0.014) | 0 | (0-0.116) | 3.9167E−01 | 1.000E+00 |
| KYNURENINE | 0.5 | (0.4-0.6) | 0 | (0-0) | 0.022 | (0-0.079) | 4.8749E−01 | 1.000E+00 |
| URATE | 0.48 | (0.38-0.58) | 0.026 | (0-0.079) | 0.082 | (0.041-0.176) | 3.6779E−01 | 1.000E+00 |
| L-GLUTAMIC ACID | 0.51 | (0.41-0.61) | 0 | (0-0) | 0.037 | (0-0.146) | 4.4204E−01 | 1.000E+00 |
| Hexaethylene glycol_contaminant | 0.48 | (0.37-0.59) | 0 | (0-0) | 0.03 | (0.007-0.071) | 3.5397E−01 | 1.000E+00 |
| CORTISOL | 0.51 | (0.4-0.62) | 0 | (0-0.105) | 0.015 | (0-0.079) | 4.2318E−01 | 1.000E+00 |
| L-RHAMNOSE | 0.49 | (0.38-0.6) | 0 | (0-0.053) | 0 | (0-0.134) | 4.1022E−01 | 1.000E+00 |
| THIAMINE | 0.48 | (0.37-0.58) | 0.026 | (0-0.079) | 0.03 | (0.007-0.105) | 3.3360E−01 | 1.000E+00 |
| L-2-PHOSPHOGLYCERIC | 0.57 | (0.46-0.67) | 0.105 | (0-0.211) | 0.052 | (0.015-0.143) | 9.9178E−02 | 1.000E+00 |
| ACID_breakthrough | | | | | | | | |
| INDOLE-3-ACETALDEHYDE | 0.49 | (0.38-0.6) | 0 | (0-0.053) | 0 | (0-0.064) | 4.1022E−01 | 1.000E+00 |
| 5′-DEOXYADENOSINE | 0.55 | (0.45-0.66) | 0.026 | (0-0.079) | 0.015 | (0-0.135) | 1.5727E−01 | 1.000E+00 |
| 5′-METHYLTHIOADENOSINE | 0.47 | (0.36-0.57) | 0 | (0-0) | 0.007 | (0-0.082) | 2.6458E−01 | 1.000E+00 |
| D-(−)-3-PHOSPHOGLYCERIC ACID | 0.5 | (0.4-0.6) | 0 | (0-0.119) | 0.03 | (0-0.127) | 4.9632E−01 | 1.000E+00 |
| 3-DEHYDROSHIKIMATE | 0.5 | (0.4-0.6) | 0 | (0-0.079) | 0.052 | (0.022-0.104) | 4.9632E−01 | 1.000E+00 |
| N-ACETYL-L-ALANINE | 0.45 | (0.33-0.56) | 0 | (0-0.04) | 0.007 | (0-0.06) | 1.6631E−01 | 1.000E+00 |
| Prostaglandin E2 | 0.56 | (0.45-0.66) | 0 | (0-0.053) | 0.007 | (0-0.214) | 1.5027E−01 | 1.000E+00 |
| D-PANTOTHENIC ACID | 0.6 | (0.5-0.7) | 0 | (0-0.079) | 0.119 | (0.063-0.194) | 3.0528E−02 | 1.000E+00 |
| 2′-DEOXYGUANOSINE 5′- | 0.5 | (0.39-0.6) | 0.026 | (0-0.105) | 0.09 | (0.052-0.142) | 4.7866E−01 | 1.000E+00 |
| MONOPHOSPHATE | | | | | | | | |
| ADENOSINE 5′-MONOPHOSPHATE | 0.47 | (0.36-0.57) | 0.026 | (0-0.145) | 0.056 | (0.018-0.153) | 2.6700E−01 | 1.000E+00 |
| EPINEPHRINE | 0.51 | (0.4-0.61) | 0.026 | (0-0.105) | 0.045 | (0.007-0.119) | 4.5226E−01 | 1.000E+00 |
| 6-HYDROXYNICOTINATE | 0.53 | (0.43-0.63) | 0 | (0-0.079) | 0.097 | (0.045-0.168) | 3.0075E−01 | 1.000E+00 |
| 2-Phenylethanol glucuronide | 0.52 | (0.42-0.63) | 0 | (0-0.079) | 0 | (0-0.108) | 3.4441E−01 | 1.000E+00 |
| Sphingomyelin(36:1) | 0.49 | (0.39-0.6) | 0 | (0-0.093) | 0.03 | (0-0.157) | 4.4934E−01 | 1.000E+00 |
| 3-HYDROXY-3-METHYLGLUTARATE | 0.56 | (0.46-0.66) | 0 | (0-0.105) | 0.067 | (0.03-0.172) | 1.3054E−01 | 1.000E+00 |
| 11-Hydroxy-9-tridecenoic acid | 0.55 | (0.44-0.65) | 0.026 | (0-0.079) | 0.022 | (0.004-0.139) | 1.9036E−01 | 1.000E+00 |
| Acylcarnitine(C16:0) | 0.52 | (0.42-0.63) | 0 | (0-0.053) | 0.052 | (0.015-0.138) | 3.4441E−01 | 1.000E+00 |
| 1-AMINOCYCLOPROPANE-1- | 0.48 | (0.37-0.59) | 0 | (0-0) | 0.045 | (0.015-0.075) | 3.5123E−01 | 1.000E+00 |
| CARBOXYLATE | | | | | | | | |
| GLUCONIC ACID | 0.56 | (0.45-0.67) | 0.026 | (0-0.093) | 0.086 | (0.037-0.157) | 1.2438E−01 | 1.000E+00 |
| URIDINE-5-MONOPHOSPHATE | 0.54 | (0.43-0.64) | 0.013 | (0-0.105) | 0.052 | (0.018-0.116) | 2.5382E−01 | 1.000E+00 |
| Phosphatidylethanolamine(34:1) | 0.51 | (0.41-0.61) | 0 | (0-0.04) | 0.045 | (0.015-0.124) | 4.2752E−01 | 1.000E+00 |
| N-ACETYL-L-LEUCINE | 0.53 | (0.43-0.63) | 0 | (0-0) | 0.015 | (0-0.214) | 3.1372E−01 | 1.000E+00 |
| GUANOSINE 5′-MONOPHOSPHATE | 0.48 | (0.38-0.58) | 0.026 | (0-0.079) | 0.067 | (0.03-0.194) | 3.5535E−01 | 1.000E+00 |
| EDTA_exogenous | 0.54 | (0.44-0.64) | 0.026 | (0-0.105) | 0.052 | (0.018-0.213) | 2.2965E−01 | 1.000E+00 |
| 6-keto Prostaglandin F1a | 0.54 | (0.43-0.65) | 0 | (0-0.105) | 0.075 | (0.033-0.134) | 2.1535E−01 | 1.000E+00 |
| 3-METHYGLUTARIC ACID | 0.51 | (0.42-0.61) | 0.026 | (0-0.105) | 0.052 | (0.018-0.165) | 3.9451E−01 | 1.000E+00 |
| TAURINE | 0.5 | (0.39-0.6) | 0 | (0-0.079) | 0.022 | (0-0.093) | 4.8307E−01 | 1.000E+00 |
| L-PHENYLALANINE | 0.48 | (0.37-0.59) | 0 | (0-0.079) | 0.037 | (0.007-0.071) | 3.5123E−01 | 1.000E+00 |
| Hydroxybutyrylcarnitine | 0.42 | (0.31-0.53) | 0 | (0-0.093) | 0 | (0-0.008) | 6.6766E−02 | 1.000E+00 |
| Hexanoic Acid | 0.57 | (0.47-0.68) | 0 | (0-0.026) | 0 | (0-0.12) | 8.4592E−02 | 1.000E+00 |
| cis-Quinceoxepane [M + Na]+ | 0.5 | (0.39-0.61) | 0 | (0-0.026) | 0 | (0-0.022) | 4.9926E−01 | 1.000E+00 |
| PYRIDOXINE | 0.52 | (0.41-0.62) | 0.026 | (0-0.093) | 0 | (0-0.112) | 3.8178E−01 | 1.000E+00 |
| RIBOFLAVIN | 0.5 | (0.39-0.61) | 0 | (0-0) | 0.075 | (0.037-0.116) | 5.0000E−01 | 1.000E+00 |
| Glutarylcarnitine [M − H2O + H]+ | 0.47 | (0.36-0.57) | 0 | (0-0) | 0 | (0-0.104) | 2.5977E−01 | 1.000E+00 |
| Free Fatty Acid (14:1) (myristoleic acid) | 0.57 | (0.46-0.67) | 0 | (0-0) | 0.06 | (0.022-0.153) | 1.1057E−01 | 1.000E+00 |
| GUANOSINE 5′-DIPHOSPHATE | 0.49 | (0.38-0.59) | 0 | (0-0.026) | 0.082 | (0.037-0.142) | 4.0878E−01 | 1.000E+00 |
| L-SERINE | 0.53 | (0.43-0.64) | 0.026 | (0-0.079) | 0.03 | (0.007-0.142) | 2.7433E−01 | 1.000E+00 |
| CREATININE | 0.53 | (0.42-0.64) | 0.026 | (0-0.093) | 0.015 | (0-0.139) | 2.8426E−01 | 1.000E+00 |
| L-CYSTEINE | 0.54 | (0.44-0.64) | 0 | (0-0.054) | 0.022 | (0-0.239) | 2.2519E−01 | 1.000E+00 |
| XANTHINE | 0.51 | (0.41-0.61) | 0.026 | (0-0.105) | 0.067 | (0.022-0.187) | 4.0449E−01 | 1.000E+00 |
| ALLANTOIN | 0.46 | (0.36-0.56) | 0 | (0-0.053) | 0.037 | (0.007-0.097) | 2.2630E−01 | 1.000E+00 |
| Acylcarnitine(C6:0) | 0.45 | (0.35-0.55) | 0 | (0-0.079) | 0.022 | (0-0.116) | 1.9237E−01 | 1.000E+00 |
| INOSINE 5′-MONOPHOSPHATE | 0.5 | (0.4-0.59) | 0 | (0-0) | 0.134 | (0.071-0.183) | 4.7131E−01 | 1.000E+00 |
| CYTIDINE 5′-DIPHOSPHOCHOLINE | 0.49 | (0.39-0.6) | 0 | (0-0.053) | 0.015 | (0-0.134) | 4.4642E−01 | 1.000E+00 |
| Acetylcarnitine | 0.54 | (0.44-0.64) | 0 | (0-0.119) | 0.037 | (0.007-0.21) | 2.2298E−01 | 1.000E+00 |
| N-ACETYL-L-PHENYLALANINE | 0.57 | (0.46-0.67) | 0 | (0-0.014) | 0.03 | (0.004-0.086) | 1.0713E−01 | 1.000E+00 |
| STACHYOSE | 0.54 | (0.43-0.64) | 0.026 | (0-0.158) | 0.127 | (0.075-0.201) | 2.4678E−01 | 1.000E+00 |
| THYROTROPIN RELEASING | 0.5 | (0.4-0.61) | 0 | (0-0.053) | 0.022 | (0.007-0.064) | 4.6544E−01 | 1.000E+00 |
| HORMONE | | | | | | | | |
| TAUROLITHOCHOLATE | 0.48 | (0.38-0.58) | 0 | (0-0.053) | 0 | (0-0.168) | 3.5948E−01 | 1.000E+00 |
| ADENOSINE 3′.5′-DIPHOSPHATE | 0.48 | (0.38-0.59) | 0 | (0-0.079) | 0.052 | (0.022-0.172) | 3.6501E−01 | 1.000E+00 |
| 2-HYDROXYPHENYLACETIC ACID | 0.52 | (0.42-0.62) | 0 | (0-0) | 0.06 | (0.022-0.146) | 3.7058E−01 | 1.000E+00 |
| N-FORMYLGLYCINE | 0.51 | (0.4-0.63) | 0.026 | (0-0.132) | 0.052 | (0.022-0.139) | 3.9025E−01 | 1.000E+00 |
| THYROXINE | 0.53 | (0.41-0.64) | 0 | (0-0.053) | 0.015 | (0-0.071) | 3.0332E−01 | 1.000E+00 |
| Chavicol O-beta-glucopyranoside | 0.56 | (0.45-0.66) | 0 | (0-0) | 0.026 | (0-0.149) | 1.3853E−01 | 1.000E+00 |
| [M − H2O + H]+ | | | | | | | | |
| N-ACETYL-D-TRYPTOPHAN | 0.54 | (0.44-0.64) | 0.026 | (0-0.093) | 0.09 | (0.037-0.134) | 2.2298E−01 | 1.000E+00 |
| XANTHOSINE 5′-MONOPHOSPHATE | 0.46 | (0.35-0.57) | 0.026 | (0-0.132) | 0.022 | (0-0.071) | 2.3415E−01 | 1.000E+00 |
| MEVALOLACTONE | 0.52 | (0.42-0.62) | 0 | (0-0.053) | 0.112 | (0.075-0.201) | 3.6779E−01 | 1.000E+00 |
| 5-VALEROLACTONE | 0.48 | (0.37-0.6) | 0 | (0-0.079) | 0 | (0-0.03) | 3.8742E−01 | 1.000E+00 |
| TRYPTOPHAN_In Source | 0.57 | (0.47-0.67) | 0 | (0-0) | 0.015 | (0-0.127) | 8.6904E−02 | 1.000E+00 |
| ETHYLMALONIC ACID | 0.48 | (0.37-0.59) | 0.026 | (0-0.093) | 0 | (0-0.082) | 3.7757E−01 | 1.000E+00 |
| NICOTINAMIDE MONONUCLEOTIDE | 0.5 | (0.39-0.6) | 0 | (0-0) | 0 | (0-0.123) | 4.8013E−01 | 1.000E+00 |
| N-Undecanoylglycine | 0.52 | (0.42-0.63) | 0 | (0-0) | 0 | (0-0.127) | 3.2161E−01 | 1.000E+00 |
| ADIPIC ACID | 0.55 | (0.45-0.66) | 0 | (0-0) | 0.052 | (0.007-0.154) | 1.5995E−01 | 1.000E+00 |
| 2′-DEOXYGUANOSINE 5′- | 0.52 | (0.42-0.62) | 0 | (0-0.079) | 0.03 | (0-0.142) | 3.6779E−01 | 1.000E+00 |
| TRIPHOSPHATE | | | | | | | | |
| D-(+)-GALACTURONIC ACID | 0.5 | (0.4-0.61) | 0.026 | (0-0.105) | 0.075 | (0.03-0.146) | 4.7572E−01 | 1.000E+00 |
| GUANOSINE 3′.5′-CYCLIC | 0.57 | (0.46-0.67) | 0 | (0-0.145) | 0.056 | (0.015-0.131) | 1.0112E−01 | 1.000E+00 |
| MONOPHOSPHATE | | | | | | | | |
| L-TYROSINE | 0.45 | (0.35-0.56) | 0 | (0-0.079) | 0.015 | (0-0.101) | 1.9036E−01 | 1.000E+00 |
| N-ACETYL-DL-SERINE | 0.38 | (0.28-0.48) | 0 | (0-0) | 0.015 | (0-0.064) | 1.3334E−02 | 1.000E+00 |
| LEUCINE | 0.56 | (0.46-0.66) | 0 | (0-0.053) | 0.037 | (0.007-0.185) | 1.1552E−01 | 1.000E+00 |
| OROTATE | 0.46 | (0.35-0.57) | 0 | (0-0.079) | 0.045 | (0.015-0.082) | 2.0893E−01 | 1.000E+00 |
| 5.6-DIHYDROURACIL | 0.45 | (0.34-0.55) | 0 | (0-0.053) | 0.037 | (0.007-0.097) | 1.5550E−01 | 1.000E+00 |
| URIDINE | 0.48 | (0.37-0.58) | 0.026 | (0-0.079) | 0.007 | (0-0.19) | 3.2161E−01 | 1.000E+00 |
| nicotinate beta-D-ribonucleotide | 0.5 | (0.4-0.61) | 0 | (0-0.066) | 0 | (0-0.15) | 4.7572E−01 | 1.000E+00 |
| Lysophosphatidylcholine(20:0) | 0.48 | (0.38-0.58) | 0.026 | (0-0.079) | 0.007 | (0-0.097) | 3.7897E−01 | 1.000E+00 |
| 1-OLEOYL-RAC-GLYCEROL | 0.49 | (0.39-0.6) | 0.026 | (0-0.119) | 0.052 | (0.022-0.157) | 4.5811E−01 | 1.000E+00 |
| 2-aminobenzamide | 0.51 | (0.41-0.62) | 0.026 | (0-0.119) | 0.045 | (0.015-0.086) | 3.9025E−01 | 1.000E+00 |
| BILIVERDIN | 0.43 | (0.34-0.53) | 0 | (0-0.026) | 0.03 | (0.007-0.146) | 1.0849E−01 | 1.000E+00 |
| ADENOSINE 5′-DIPHOSPHORIBOSE | 0.5 | (0.39-0.6) | 0 | (0-0) | 0.007 | (0-0.18) | 4.8013E−01 | 1.000E+00 |
| AZELAIC ACID | 0.53 | (0.42-0.63) | 0 | (0-0.026) | 0.045 | (0.018-0.127) | 2.9308E−01 | 1.000E+00 |
| ROSMARINIC ACID | 0.53 | (0.41-0.64) | 0 | (0-0.066) | 0.022 | (0-0.071) | 3.0461E−01 | 1.000E+00 |
| DEOXYCYTIDINE | 0.54 | (0.44-0.65) | 0.026 | (0-0.079) | 0.034 | (0.004-0.191) | 2.0787E−01 | 1.000E+00 |
| Indoleacetaldehyde | 0.54 | (0.44-0.65) | 0 | (0-0.026) | 0.052 | (0.015-0.119) | 2.0787E−01 | 1.000E+00 |
| HYPOTAURINE_second peak | 0.57 | (0.46-0.68) | 0.026 | (0-0.119) | 0.075 | (0.03-0.123) | 9.2279E−02 | 1.000E+00 |
| 3-HYDROXYBENZYL ALCOHOL | 0.45 | (0.34-0.55) | 0 | (0-0) | 0.007 | (0-0.06) | 1.6175E−01 | 1.000E+00 |
| 3-Dehydrocarnitine | 0.43 | (0.32-0.53) | 0 | (0-0.079) | 0.015 | (0-0.045) | 8.8669E−02 | 1.000E+00 |
| NORADRENALINE | 0.53 | (0.43-0.63) | 0 | (0-0.026) | 0.03 | (0.007-0.112) | 2.7804E−01 | 1.000E+00 |
| S-CARBOXYMETHYL-L-CYSTEINE | 0.51 | (0.41-0.61) | 0 | (0-0) | 0.101 | (0.048-0.153) | 4.2607E−01 | 1.000E+00 |
| ISOCITRIC ACID | 0.47 | (0.36-0.57) | 0.026 | (0-0.093) | 0.045 | (0.011-0.157) | 2.6217E−01 | 1.000E+00 |
| ALPHA-AMINOADIPATE | 0.49 | (0.39-0.6) | 0 | (0-0.04) | 0.007 | (0-0.06) | 4.4934E−01 | 1.000E+00 |
| Acylcarnitine(C18:2n-6) | 0.46 | (0.36-0.57) | 0 | (0-0.066) | 0.045 | (0.015-0.146) | 2.4100E−01 | 1.000E+00 |
| Acylcarnitine(C12:0) | 0.47 | (0.36-0.58) | 0 | (0-0.079) | 0.007 | (0-0.06) | 2.8176E−01 | 1.000E+00 |
| 8-Deoxy-11,13-dihydroxygrosheimin | 0.54 | (0.43-0.64) | 0 | (0-0.04) | 0.045 | (0.015-0.093) | 2.3642E−01 | 1.000E+00 |
| 2′-DEOXYURIDINE 5′-MONO-PHOS- | 0.46 | (0.36-0.56) | 0 | (0-0) | 0.015 | (0-0.086) | 2.3985E−01 | 1.000E+00 |
| PHATE | | | | | | | | |
| PYRIDOXAL 5′-PHOSPHATE | 0.41 | (0.31-0.52) | 0.026 | (0-0.093) | 0.03 | (0.007-0.093) | 5.1995E−02 | 1.000E+00 |
| L-PIPECOLIC ACID | 0.51 | (0.4-0.61) | 0 | (0-0) | 0 | (0-0.007) | 4.3477E−01 | 1.000E+00 |
| D-RIBULOSE 1.5-BISPHOSPHATE | 0.43 | (0.33-0.54) | 0.026 | (0-0.079) | 0.015 | (0-0.082) | 1.0510E−01 | 1.000E+00 |
| URIDINE 5′-DIPHOSPHOGLUCURONIC | 0.52 | (0.41-0.63) | 0.026 | (0-0.132) | 0.037 | (0.007-0.086) | 3.5948E−01 | 1.000E+00 |
| ACID | | | | | | | | |
| LUMICHROME | 0.49 | (0.37-0.6) | 0.026 | (0-0.119) | 0 | (0-0.022) | 3.9735E−01 | 1.000E+00 |
| N-ACETYL-DL-METHIONINE | 0.54 | (0.43-0.64) | 0.026 | (0-0.145) | 0.075 | (0.033-0.179) | 2.4330E−01 | 1.000E+00 |
| OMEGA-HYDROXYDODECANOIC | 0.47 | (0.37-0.57) | 0 | (0-0) | 0 | (0-0.075) | 2.8803E−01 | 1.000E+00 |
| ACID | | | | | | | | |
| GLYCERALDEHYDE | 0.57 | (0.46-0.67) | 0 | (0-0.026) | 0.045 | (0.011-0.127) | 1.0781E−01 | 1.000E+00 |
| Acetylsalicyclic acid | 0.41 | (0.31-0.51) | 0 | (0-0.066) | 0.03 | (0.007-0.067) | 4.9681E−02 | 1.000E+00 |
| 2,6-diisopropylnapthalene [M + Na]+ | 0.53 | (0.42-0.63) | 0 | (0-0.053) | 0 | (0-0.079) | 3.1241E−01 | 1.000E+00 |
| 1-(sn-Glycero-3-phosphate)-1D-myo- | 0.48 | (0.38-0.58) | 0 | (0-0) | 0.09 | (0.045-0.142) | 3.5123E−01 | 1.000E+00 |
| inositol | | | | | | | | |
| PHENYLETHANOLAMINE | 0.45 | (0.35-0.55) | 0 | (0-0) | 0 | (0-0.153) | 1.8342E−01 | 1.000E+00 |
| Lauroylcarnitine_break through | 0.47 | (0.36-0.57) | 0 | (0-0.053) | 0.03 | (0-0.113) | 2.6579E−01 | 1.000E+00 |
| Free Fatty Acid (16:0) (palmitic acid) | 0.43 | (0.32-0.54) | 0 | (0-0) | 0 | (0-0.011) | 1.0577E−01 | 1.000E+00 |
| HYPOTAURINE | 0.53 | (0.42-0.64) | 0.026 | (0-0.093) | 0.015 | (0-0.104) | 2.8803E−01 | 1.000E+00 |
| THYMIDINE | 0.49 | (0.39-0.59) | 0 | (0-0.079) | 0.037 | (0.007-0.131) | 4.3913E−01 | 1.000E+00 |
| PYRIDOXAL | 0.56 | (0.46-0.67) | 0.053 | (0-0.132) | 0.03 | (0.007-0.139) | 1.1481E−01 | 1.000E+00 |
| ALPHA-KETOGLUTARIC ACID | 0.5 | (0.4-0.61) | 0 | (0-0.066) | 0.104 | (0.052-0.164) | 4.7572E−01 | 1.000E+00 |
| O-SUCCINYL-L-HOMOSERINE | 0.51 | (0.4-0.62) | 0.026 | (0-0.172) | 0.015 | (0-0.093) | 4.2462E−01 | 1.000E+00 |
| O-ACETYL-L-CARNITINE | 0.48 | (0.37-0.59) | 0 | (0-0) | 0.022 | (0-0.094) | 3.5810E−01 | 1.000E+00 |
| 3.4-DIHYDROXYPHENYL GLYCOL | 0.47 | (0.37-0.58) | 0.026 | (0-0.105) | 0.007 | (0-0.101) | 3.1110E−01 | 1.000E+00 |
| N′-nitrosonornicotine | 0.37 | (0.26-0.47) | 0 | (0-0) | 0 | (0-0.052) | 6.0092E−03 | 1.000E+00 |
| 4-Acetamido-2-aminobutanoic acid | 0.43 | (0.33-0.54) | 0.026 | (0-0.093) | 0 | (0-0.06) | 1.0510E−01 | 1.000E+00 |
| sterol | 0.49 | (0.39-0.6) | 0.026 | (0-0.119) | 0.015 | (0-0.06) | 4.4934E−01 | 1.000E+00 |
| INDOLE-3-PYRUVIC ACID | 0.49 | (0.38-0.59) | 0 | (0-0.079) | 0 | (0-0.064) | 4.2029E−01 | 1.000E+00 |
| Octanoic Acid | 0.48 | (0.37-0.58) | 0 | (0-0) | 0.034 | (0.007-0.079) | 3.3226E−01 | 1.000E+00 |
| ASCORBATE | 0.51 | (0.41-0.62) | 0.026 | (0-0.066) | 0.045 | (0.015-0.09) | 3.9451E−01 | 1.000E+00 |
| N1-ACETYLSPERMINE | 0.49 | (0.39-0.59) | 0.026 | (0-0.079) | 0.037 | (0.007-0.075) | 4.3622E−01 | 1.000E+00 |
| Propionylcarnitine | 0.45 | (0.35-0.55) | 0 | (0-0.026) | 0.06 | (0.03-0.134) | 1.7856E−01 | 1.000E+00 |
| L-TRYPTOPHANAMIDE | 0.55 | (0.44-0.65) | 0.053 | (0-0.158) | 0.082 | (0.026-0.187) | 1.8539E−01 | 1.000E+00 |
| GUANINE | 0.53 | (0.43-0.63) | 0 | (0-0.066) | 0.022 | (0-0.105) | 2.9308E−01 | 1.000E+00 |
| D-2-Hydroxyglutarate | 0.48 | (0.38-0.59) | 0 | (0-0.066) | 0.03 | (0.007-0.075) | 3.6224E−01 | 1.000E+00 |
| CORTISOL 21-ACETATE | 0.47 | (0.36-0.59) | 0 | (0-0.079) | 0 | (0-0.049) | 2.9308E−01 | 1.000E+00 |
| 3.4-DIHYDROXYBENZOATE | 0.45 | (0.34-0.56) | 0 | (0-0.145) | 0 | (0-0.03) | 1.8440E−01 | 1.000E+00 |
| Cholesterol glucuronide | 0.46 | (0.35-0.56) | 0 | (0-0) | 0.045 | (0.007-0.079) | 2.1320E−01 | 1.000E+00 |
| Lysophosphatidylcholine(20:1) | 0.48 | (0.38-0.58) | 0 | (0-0) | 0.03 | (0.007-0.142) | 3.5810E−01 | 1.000E+00 |
| D-RIBOSE 5-PHOSPHATE | 0.49 | (0.39-0.59) | 0 | (0-0) | 0.022 | (0.004-0.079) | 4.3768E−01 | 1.000E+00 |
| methionine sulfoxide | 0.5 | (0.39-0.6) | 0.026 | (0-0.105) | 0 | (0-0.064) | 4.7425E−01 | 1.000E+00 |
| PHOSPHOCHOLINE | 0.41 | (0.31-0.52) | 0 | (0-0.066) | 0.045 | (0.015-0.09) | 5.3585E−02 | 1.000E+00 |
| sn-glycero-3-Phosphoethanolamine | 0.48 | (0.38-0.59) | 0 | (0-0.066) | 0.03 | (0.004-0.09) | 3.8038E−01 | 1.000E+00 |
| Prostaglandin D2 | 0.48 | (0.37-0.59) | 0 | (0-0) | 0 | (0-0.015) | 3.5260E−01 | 1.000E+00 |
| THYMINE | 0.55 | (0.46-0.65) | 0 | (0-0.053) | 0.104 | (0.052-0.265) | 1.5905E−01 | 1.000E+00 |
| THIOPURINE S-METHYLETHER | 0.46 | (0.36-0.56) | 0 | (0-0.066) | 0.067 | (0.022-0.112) | 2.3756E−01 | 1.000E+00 |
| THIOUREA | 0.5 | (0.4-0.61) | 0.026 | (0-0.119) | 0.034 | (0.015-0.079) | 4.6544E−01 | 1.000E+00 |
| Oleamide | 0.47 | (0.37-0.57) | 0 | (0-0) | 0.03 | (0-0.172) | 2.7928E−01 | 1.000E+00 |
| Free Fatty Acid (20:4) (arachidonic acid) | 0.47 | (0.36-0.58) | 0 | (0-0.079) | 0 | (0-0.045) | 3.0075E−01 | 1.000E+00 |
| PTERIN | 0.49 | (0.39-0.59) | 0 | (0-0) | 0 | (0-0.097) | 4.3768E−01 | 1.000E+00 |
| N-ACETYLPUTRESCINE | 0.52 | (0.42-0.63) | 0.053 | (0-0.172) | 0.037 | (0.015-0.082) | 3.4850E−01 | 1.000E+00 |
| simvastatin hydroxy acid | 0.47 | (0.36-0.57) | 0 | (0-0) | 0.015 | (0-0.093) | 2.5858E−01 | 1.000E+00 |
| L-VALINE | 0.56 | (0.46-0.67) | 0 | (0-0.014) | 0.03 | (0-0.093) | 1.2287E−01 | 1.000E+00 |
| Pentanoic Acid | 0.46 | (0.35-0.56) | 0.026 | (0-0.119) | 0.06 | (0.026-0.119) | 2.2079E−01 | 1.000E+00 |
| CUDA_istd | 0.51 | (0.41-0.61) | 0 | (0-0) | 0 | (0-0.079) | 4.0020E−01 | 1.000E+00 |
| Aspartyl-Threonine | 0.43 | (0.33-0.54) | 0 | (0-0.079) | 0.015 | (0-0.071) | 1.0781E−01 | 1.000E+00 |
| N-AMIDINO-L-ASPARTATE | 0.41 | (0.31-0.5) | 0 | (0-0.026) | 0.015 | (0-0.108) | 3.8384E−02 | 1.000E+00 |
| 4-GUANIDINOBUTANOATE | 0.42 | (0.31-0.54) | 0 | (0-0.053) | 0.007 | (0-0.071) | 7.4235E−02 | 1.000E+00 |
| HEPTANOIC ACID | 0.49 | (0.38-0.59) | 0 | (0-0.105) | 0.007 | (0-0.079) | 3.9309E−01 | 1.000E+00 |
| 4-IMIDAZOLEACETIC ACID | 0.49 | (0.39-0.6) | 0 | (0-0.079) | 0.015 | (0-0.097) | 4.5811E−01 | 1.000E+00 |
| L-METHIONINE | 0.49 | (0.38-0.59) | 0 | (0-0.079) | 0 | (0-0.093) | 3.9309E−01 | 1.000E+00 |
| 3-HYDROXYBUTANOIC ACID | 0.56 | (0.46-0.66) | 0 | (0-0.026) | 0.03 | (0-0.19) | 1.4432E−01 | 1.000E+00 |
| D-GLUCOSAMINE 6-PHOSPHATE | 0.43 | (0.32-0.54) | 0.026 | (0-0.079) | 0.007 | (0-0.052) | 9.4124E−02 | 1.000E+00 |
| Ecgonine | 0.43 | (0.32-0.54) | 0 | (0-0.145) | 0.007 | (0-0.09) | 9.4124E−02 | 1.000E+00 |
| Propionic Acid | 0.46 | (0.36-0.55) | 0 | (0-0) | 0.022 | (0-0.079) | 2.0263E−01 | 1.000E+00 |
| Decanoic Acid | 0.43 | (0.33-0.53) | 0 | (0-0) | 0.007 | (0-0.082) | 8.7489E−02 | 1.000E+00 |
| Free Fatty Acid (18:2) (linoleic acid) | 0.45 | (0.34-0.55) | 0 | (0-0.053) | 0.015 | (0-0.105) | 1.6265E−01 | 1.000E+00 |
| Putative Glycodeoxycholic acid (GDCA) | 0.49 | (0.39-0.58) | 0 | (0-0.026) | 0.119 | (0.06-0.187) | 4.1165E−01 | 1.000E+00 |
| Acylcarnitine(C4:0) | 0.4 | (0.3-0.5) | 0 | (0-0) | 0.03 | (0.007-0.064) | 3.3715E−02 | 1.000E+00 |
| Glycodeoxycholic acid (GDCA) | 0.48 | (0.38-0.58) | 0 | (0-0) | 0.052 | (0.018-0.108) | 3.6779E−01 | 1.000E+00 |
| L-HOMOCYSTEINE THIOLACTONE | 0.48 | (0.37-0.59) | 0.026 | (0-0.079) | 0.022 | (0-0.075) | 3.4986E−01 | 1.000E+00 |
| HOMOCYSTEINE | 0.43 | (0.33-0.54) | 0.053 | (0-0.132) | 0.007 | (0-0.09) | 1.0244E−01 | 1.000E+00 |
| Hydroxyphenyllactic acid | 0.49 | (0.38-0.6) | 0.053 | (0-0.158) | 0 | (0-0.053) | 4.3477E−01 | 1.000E+00 |
| NEPSILON.NEPSILON.NEPSILON- | 0.43 | (0.33-0.54) | 0 | (0-0) | 0.015 | (0-0.082) | 1.0713E−01 | 1.000E+00 |
| TRIMETHYLLYSINE | | | | | | | | |
| URIDINE 5′-DIPHOSPHOGLUCOSE | 0.41 | (0.3-0.51) | 0 | (0-0) | 0.015 | (0-0.064) | 3.8078E−02 | 1.000E+00 |
| CORTISONE | 0.46 | (0.35-0.56) | 0 | (0-0.04) | 0 | (0-0.03) | 2.0576E−01 | 1.000E+00 |
| HIPPURATE | 0.52 | (0.42-0.61) | 0 | (0-0.04) | 0.03 | (0.007-0.284) | 3.8178E−01 | 1.000E+00 |
| CAFFEINE | 0.49 | (0.38-0.6) | 0 | (0-0) | 0.007 | (0-0.164) | 4.3477E−01 | 1.000E+00 |
| NORLEUCINE | 0.45 | (0.34-0.56) | 0.026 | (0-0.132) | 0.045 | (0.015-0.112) | 1.7190E−01 | 1.000E+00 |
| 5-AMINOIMIDAZOLE-4- | 0.38 | (0.27-0.48) | 0 | (0-0) | 0 | (0-0.011) | 1.0381E−02 | 1.000E+00 |
| CARBOXAMIDE-1-BETA-D- | | | | | | | | |
| RIBOFURANOSYL 5′- | | | | | | | | |
| MONOPHOSPHATE | | | | | | | | |
| Hydroxypropionylcarnitine | 0.44 | (0.32-0.55) | 0 | (0-0.014) | 0.007 | (0-0.026) | 1.1197E−01 | 1.000E+00 |
| UROCANATE | 0.44 | (0.34-0.55) | 0.026 | (0-0.105) | 0.045 | (0.015-0.093) | 1.4099E−01 | 1.000E+00 |
| Indole-3-lactic acid | 0.4 | (0.3-0.5) | 0 | (0-0.04) | 0.022 | (0-0.09) | 3.3715E−02 | 1.000E+00 |
| Free Fatty Acid (20:5) (eicosapentaenoic | 0.44 | (0.34-0.54) | 0 | (0-0.04) | 0.015 | (0-0.097) | 1.3691E−01 | 1.000E+00 |
| acid) | | | | | | | | |
| DIMETHYLBENZIMIDAZOLE | 0.47 | (0.37-0.58) | 0 | (0-0) | 0.037 | (0.015-0.079) | 3.0850E−01 | 1.000E+00 |
| CADP-RIBOSE | 0.49 | (0.38-0.6) | 0 | (0-0.079) | 0.015 | (0-0.09) | 4.3477E−01 | 1.000E+00 |
| Eicosapentaenoic Acid (n-3) | 0.46 | (0.35-0.57) | 0.026 | (0-0.066) | 0.045 | (0.011-0.093) | 2.3756E−01 | 1.000E+00 |
| 4-HYDROXYPHENYLACETATE | 0.51 | (0.41-0.62) | 0.026 | (0-0.105) | 0.022 | (0-0.093) | 3.9167E−01 | 1.000E+00 |
| 3-AMINO-5-HYDROXYBENZOIC ACID | 0.44 | (0.34-0.54) | 0 | (0-0.066) | 0.015 | (0-0.09) | 1.3691E−01 | 1.000E+00 |
| D-GLUCONATE | 0.41 | (0.3-0.53) | 0 | (0-0.054) | 0.015 | (0-0.045) | 5.3184E−02 | 1.000E+00 |
| D-ASPARTATE | 0.39 | (0.28-0.5) | 0 | (0-0) | 0.007 | (0-0.034) | 2.2430E−02 | 1.000E+00 |
| Thromboxane B2 | 0.53 | (0.41-0.64) | 0 | (0-0.066) | 0 | (0-0.112) | 3.1110E−01 | 1.000E+00 |
| MELIBIOSE | 0.48 | (0.38-0.58) | 0 | (0-0.079) | 0 | (0-0.15) | 3.4305E−01 | 1.000E+00 |
| 4-HYDROXYBENZALDEHYDE | 0.48 | (0.38-0.58) | 0 | (0-0.026) | 0.09 | (0.056-0.168) | 3.4305E−01 | 1.000E+00 |
| ALPHA-D-GALACTOSE 1-PHOSPHATE | 0.45 | (0.33-0.56) | 0.053 | (0-0.132) | 0.015 | (0-0.06) | 1.6265E−01 | 1.000E+00 |
| 3′-CMP | 0.46 | (0.37-0.56) | 0.026 | (0-0.079) | 0.067 | (0.022-0.209) | 2.5501E−01 | 1.000E+00 |
| RETINOATE | 0.49 | (0.39-0.6) | 0 | (0-0) | 0.03 | (0.007-0.149) | 4.4496E−01 | 1.000E+00 |
| 1-(2,3-Dihydro-1H-pyrrolizin-5-yl)-1,4- | 0.43 | (0.33-0.54) | 0 | (0-0.079) | 0.045 | (0.011-0.108) | 1.0849E−01 | 1.000E+00 |
| pentanedione | | | | | | | | |
| CYTIDINE 5′-TRIPHOSPHATE | 0.44 | (0.34-0.54) | 0.026 | (0-0.105) | 0 | (0-0.071) | 1.2287E−01 | 1.000E+00 |
| 3-METHYL-2-OXINDOLE | 0.4 | (0.3-0.5) | 0.026 | (0-0.093) | 0 | (0-0.082) | 3.6280E−02 | 1.000E+00 |
| DEOXYCORTICOSTERONE ACETATE | 0.45 | (0.35-0.54) | 0 | (0-0) | 0.082 | (0.03-0.205) | 1.5550E−01 | 1.000E+00 |
| L-2-PHOSPHOGLYCERIC ACID | 0.53 | (0.42-0.64) | 0 | (0-0.079) | 0 | (0-0.026) | 2.8551E−01 | 1.000E+00 |
| 6-PHOSPHOGLUCONIC ACID | 0.54 | (0.43-0.65) | 0 | (0-0) | 0.007 | (0-0.095) | 2.3756E−01 | 1.000E+00 |
| CITRAMALATE | 0.44 | (0.34-0.54) | 0 | (0-0.053) | 0.022 | (0.007-0.082) | 1.2287E−01 | 1.000E+00 |
| HYDROQUINONE | 0.48 | (0.38-0.58) | 0 | (0-0) | 0.015 | (0-0.134) | 3.5535E−01 | 1.000E+00 |
| Alloxan [M + NH4]+; TRIGONELLINE | 0.43 | (0.33-0.53) | 0 | (0-0.014) | 0.052 | (0.022-0.104) | 1.0376E−01 | 1.000E+00 |
| GLYCOCHOLATE | 0.42 | (0.32-0.52) | 0 | (0-0) | 0.015 | (0-0.079) | 6.3486E−02 | 1.000E+00 |
| THYMIDINE 5′-MONOPHOSPHATE | 0.54 | (0.43-0.65) | 0.026 | (0-0.079) | 0.075 | (0.037-0.12) | 2.2188E−01 | 1.000E+00 |
| Glycyl-threonine | 0.41 | (0.29-0.52) | 0 | (0-0.079) | 0.045 | (0.015-0.082) | 3.7773E−02 | 1.000E+00 |
| CAFFEATE | 0.45 | (0.35-0.55) | 0 | (0-0) | 0.045 | (0.022-0.12) | 1.7569E−01 | 1.000E+00 |
| 5,6-dihydrouridine | 0.49 | (0.38-0.6) | 0 | (0-0.04) | 0.037 | (0.007-0.09) | 4.0306E−01 | 1.000E+00 |
| INDOLE-3-ACETAMIDE | 0.4 | (0.3-0.51) | 0 | (0-0) | 0 | (0-0.011) | 3.6872E−02 | 1.000E+00 |
| Metformin | 0.44 | (0.33-0.54) | 0 | (0-0.053) | 0.022 | (0-0.119) | 1.1197E−01 | 1.000E+00 |
| DIETHYL 2-METHYL-3- | 0.47 | (0.37-0.57) | 0 | (0-0) | 0.022 | (0-0.131) | 2.9308E−01 | 1.000E+00 |
| OXOSUCCINATE | | | | | | | | |
| INDOLE | 0.44 | (0.33-0.56) | 0.026 | (0-0.079) | 0.007 | (0-0.052) | 1.4941E−01 | 1.000E+00 |
| MALEIC ACID | 0.47 | (0.37-0.58) | 0.039 | (0-0.172) | 0.045 | (0.015-0.104) | 3.1110E−01 | 1.000E+00 |
| L-GLUTAMIC ACID_In Source | 0.41 | (0.31-0.51) | 0 | (0-0) | 0.022 | (0.007-0.06) | 5.0443E−02 | 1.000E+00 |
| N-ACETYL-DL-SERINE_breakthrough | 0.41 | (0.29-0.52) | 0 | (0-0.014) | 0.007 | (0-0.037) | 3.7773E−02 | 1.000E+00 |
| Sphingomyelin(32:1) | 0.49 | (0.39-0.59) | 0 | (0-0.053) | 0.049 | (0.007-0.216) | 4.1597E−01 | 1.000E+00 |
| D-GLYCERIC ACID | 0.48 | (0.37-0.58) | 0 | (0-0) | 0.011 | (0-0.131) | 3.2161E−01 | 1.000E+00 |
| S-(5′-ADENOSYL)-L-HOMOCYSTEINE | 0.43 | (0.33-0.53) | 0 | (0-0.079) | 0.015 | (0-0.148) | 1.0376E−01 | 1.000E+00 |
| 2-QUINOLINECARBOXYLIC ACID | 0.49 | (0.39-0.59) | 0 | (0-0.026) | 0.052 | (0.018-0.243) | 4.4934E−01 | 1.000E+00 |
| HOMOSERINE | 0.46 | (0.35-0.56) | 0 | (0-0.132) | 0.015 | (0-0.09) | 2.0263E−01 | 1.000E+00 |
| N-ACETYLGLYCINE | 0.49 | (0.38-0.59) | 0 | (0-0.08) | 0 | (0-0.086) | 3.9309E−01 | 1.000E+00 |
| INDOXYL SULFATE | 0.47 | (0.36-0.57) | 0 | (0-0.079) | 0.045 | (0.015-0.101) | 2.6217E−01 | 1.000E+00 |
| Nicardipine | 0.41 | (0.3-0.52) | 0 | (0-0.079) | 0.022 | (0-0.082) | 5.0828E−02 | 1.000E+00 |
| Phenacetin | 0.5 | (0.4-0.61) | 0 | (0-0) | 0.015 | (0-0.112) | 4.9043E−01 | 1.000E+00 |
| MELATONIN | 0.4 | (0.3-0.51) | 0 | (0-0) | 0.03 | (0.007-0.105) | 3.5121E−02 | 1.000E+00 |
| 3-METHOXY-L-TYROSINE | 0.4 | (0.3-0.51) | 0 | (0-0.119) | 0.015 | (0-0.064) | 3.5121E−02 | 1.000E+00 |
| PYRIXODINE_Breakthrough | 0.52 | (0.41-0.63) | 0 | (0-0.026) | 0 | (0-0.067) | 3.3226E−01 | 1.000E+00 |
| L-CYSTEIC ACID | 0.47 | (0.36-0.58) | 0 | (0-0.105) | 0.015 | (0-0.075) | 2.9690E−01 | 1.000E+00 |
| URIDINE 5′-DIPHOSPHATE | 0.43 | (0.33-0.53) | 0 | (0-0) | 0.022 | (0-0.075) | 1.0376E−01 | 1.000E+00 |
| Free Fatty Acid (22:6) (docosahexaenoic | 0.4 | (0.3-0.51) | 0 | (0-0.105) | 0.03 | (0-0.104) | 3.5121E−02 | 1.000E+00 |
| acid) | | | | | | | | |
| D-GLUCOSE-6-PHOSPHATE | 0.49 | (0.38-0.59) | 0 | (0-0.053) | 0 | (0-0.053) | 3.9309E−01 | 1.000E+00 |
| PHOSPHOCHOLINE_insource | 0.48 | (0.38-0.58) | 0 | (0-0.053) | 0.045 | (0.015-0.135) | 3.6224E−01 | 1.000E+00 |
| L-ARGININE | 0.47 | (0.36-0.58) | 0 | (0-0) | 0 | (0-0.06) | 2.7556E−01 | 1.000E+00 |
| ELAIDIC ACID | 0.43 | (0.32-0.54) | 0 | (0-0) | 0.022 | (0-0.06) | 9.0461E−02 | 1.000E+00 |
| L-PROLINE | 0.39 | (0.28-0.5) | 0 | (0-0) | 0.022 | (0-0.086) | 1.7143E−02 | 1.000E+00 |
| METHYLMALONATE | 0.48 | (0.38-0.58) | 0 | (0-0.079) | 0.015 | (0-0.119) | 3.4170E−01 | 1.000E+00 |
| hydroxyisovaleroyl carnitine | 0.4 | (0.29-0.5) | 0.026 | (0-0.132) | 0.007 | (0-0.086) | 2.5995E−02 | 1.000E+00 |
| Lysophosphatidylethanolamine(16:0) | 0.48 | (0.37-0.59) | 0.053 | (0-0.158) | 0.007 | (0-0.06) | 3.6501E−01 | 1.000E+00 |
| HOMOGENTISATE | 0.48 | (0.38-0.59) | 0 | (0-0.079) | 0.037 | (0.015-0.153) | 3.8178E−01 | 1.000E+00 |
| 5,6-dihydrouridine [M + K]+ | 0.43 | (0.32-0.54) | 0.079 | (0-0.158) | 0 | (0-0.052) | 9.7260E−02 | 1.000E+00 |
| INOSINE 5′-PHOSPHATE_breakthrough | 0.46 | (0.37-0.55) | 0 | (0-0) | 0.022 | (0-0.176) | 2.1643E−01 | 1.000E+00 |
| LAUROYLCARNITINE | 0.41 | (0.32-0.51) | 0 | (0-0) | 0 | (0-0.071) | 5.2389E−02 | 1.000E+00 |
| Indole-derivative | 0.49 | (0.38-0.6) | 0 | (0-0) | 0.007 | (0-0.075) | 4.2173E−01 | 1.000E+00 |
| INDOLE-3-ACETATE | 0.39 | (0.28-0.5) | 0 | (0-0.093) | 0.03 | (0.007-0.075) | 1.8271E−02 | 1.000E+00 |
| L-METHIONINE SULFOXIMINE | 0.5 | (0.39-0.6) | 0.026 | (0-0.145) | 0 | (0-0.101) | 4.8160E−01 | 1.000E+00 |
| Lysophosphatidylethanolamine(22:6) | 0.46 | (0.35-0.57) | 0 | (0-0.053) | 0.037 | (0.007-0.112) | 2.2741E−01 | 1.000E+00 |
| LEUKOTRIENE B4 | 0.48 | (0.37-0.58) | 0 | (0-0.053) | 0.045 | (0.015-0.142) | 3.5123E−01 | 1.000E+00 |
| 12(s)-HETE | 0.48 | (0.38-0.58) | 0.026 | (0-0.105) | 0.052 | (0.022-0.09) | 3.5810E−01 | 1.000E+00 |
| INOSINE 5′-DIPHOSPHATE | 0.43 | (0.33-0.54) | 0 | (0-0.066) | 0.015 | (0-0.105) | 9.7260E−02 | 1.000E+00 |
| CITRATE | 0.47 | (0.37-0.57) | 0 | (0-0) | 0.022 | (0-0.06) | 2.8176E−01 | 1.000E+00 |
| SUBERIC ACID | 0.41 | (0.3-0.51) | 0 | (0-0) | 0 | (0-0.041) | 3.8384E−02 | 1.000E+00 |
| 3.4-DIHYDROXYPHENYLACETATE | 0.44 | (0.34-0.54) | 0 | (0-0) | 0.015 | (0-0.097) | 1.2898E−01 | 1.000E+00 |
| FERULATE | 0.45 | (0.34-0.56) | 0 | (0-0.053) | 0.022 | (0-0.052) | 1.6631E−01 | 1.000E+00 |
| 2-HYDROXYPYRIDINE | 0.48 | (0.37-0.59) | 0 | (0-0.053) | 0.022 | (0-0.116) | 3.6501E−01 | 1.000E+00 |
| QUINATE | 0.48 | (0.38-0.57) | 0 | (0-0) | 0.06 | (0.03-0.131) | 3.3899E−01 | 1.000E+00 |
| D-GLUCURONIC ACID | 0.47 | (0.36-0.57) | 0 | (0-0.014) | 0.075 | (0.037-0.123) | 2.6579E−01 | 1.000E+00 |
| Indoleacrylic acid | 0.47 | (0.37-0.58) | 0 | (0-0) | 0.045 | (0.011-0.101) | 3.1765E−01 | 1.000E+00 |
| CHENODEOXYCHOLATE | 0.46 | (0.35-0.56) | 0 | (0-0) | 0.007 | (0-0.053) | 2.0055E−01 | 1.000E+00 |
| 3′.5′-CYCLIC AMP | 0.41 | (0.31-0.51) | 0 | (0-0) | 0 | (0-0.034) | 4.5653E−02 | 1.000E+00 |
| NICOTINAMIDE | 0.48 | (0.37-0.58) | 0 | (0-0.026) | 0 | (0-0.067) | 3.5123E−01 | 1.000E+00 |
| Ceramide(40:1) | 0.45 | (0.34-0.56) | 0.026 | (0-0.066) | 0.022 | (0-0.06) | 1.6631E−01 | 1.000E+00 |
| Acylcarnitine(C10:0) | 0.42 | (0.31-0.53) | 0 | (0-0.079) | 0.007 | (0-0.052) | 7.5803E−02 | 1.000E+00 |
| O-ACETYL-L-SERINE | 0.4 | (0.3-0.51) | 0 | (0-0) | 0.007 | (0-0.071) | 3.5987E−02 | 1.000E+00 |
| 2′.4′-DIHYDROXYACETOPHENONE | 0.35 | (0.25-0.45) | 0 | (0-0) | 0 | (0-0.071) | 2.3078E−03 | 1.000E+00 |
| N-ACETYL-DL-GLUTAMIC ACID | 0.39 | (0.29-0.48) | 0 | (0-0.079) | 0.007 | (0-0.082) | 1.7143E−02 | 1.000E+00 |
| D-GLUCONO-1.5-LACTONE | 0.43 | (0.32-0.53) | 0 | (0-0) | 0.007 | (0-0.06) | 8.2326E−02 | 1.000E+00 |
| cis-9, cis-12-Octadecadienoic acid | 0.45 | (0.35-0.55) | 0.053 | (0-0.119) | 0.052 | (0.011-0.101) | 1.9036E−01 | 1.000E+00 |
| CORTICOSTERONE | 0.48 | (0.37-0.58) | 0 | (0-0.079) | 0.007 | (0-0.022) | 3.2293E−01 | 1.000E+00 |
| 5-OXO-L-PROLINE | 0.39 | (0.28-0.49) | 0 | (0-0.014) | 0.045 | (0.015-0.093) | 1.8271E−02 | 1.000E+00 |
| DEOXYCHOLATE | 0.49 | (0.39-0.6) | 0.026 | (0-0.079) | 0.015 | (0-0.052) | 4.5811E−01 | 1.000E+00 |
| 2-AMINOPHENOL | 0.44 | (0.33-0.55) | 0.026 | (0-0.079) | 0.015 | (0-0.06) | 1.3853E−01 | 1.000E+00 |
| TRIGONELLINE | 0.45 | (0.34-0.56) | 0 | (0-0.053) | 0.019 | (0-0.097) | 1.8440E−01 | 1.000E+00 |
| TRIGONELLINE_split peak | 0.44 | (0.33-0.55) | 0 | (0-0.053) | 0.015 | (0-0.068) | 1.3853E−01 | 1.000E+00 |
| URIDINE 5′-DIPHOSPHOGALACTOSE | 0.44 | (0.34-0.55) | 0.026 | (0-0.093) | 0.03 | (0.007-0.116) | 1.3691E−01 | 1.000E+00 |
| DEOXYCARNITINE | 0.46 | (0.36-0.56) | 0 | (0-0.079) | 0.03 | (0.007-0.067) | 2.2079E−01 | 1.000E+00 |
| CHOLATE | 0.43 | (0.32-0.54) | 0 | (0-0) | 0.011 | (0-0.067) | 1.0112E−01 | 1.000E+00 |
| ALPHA-D-GLUCOSE 1-PHOSPHATE | 0.38 | (0.27-0.48) | 0.026 | (0-0.093) | 0.007 | (0-0.06) | 1.0080E−02 | 1.000E+00 |
| Myristelaidic Acid | 0.39 | (0.29-0.48) | 0 | (0-0) | 0.052 | (0.015-0.119) | 1.6076E−02 | 1.000E+00 |
| CARNOSINE | 0.37 | (0.27-0.47) | 0 | (0-0) | 0.022 | (0-0.052) | 7.8537E−03 | 1.000E+00 |
| Benzoylecgonine | 0.36 | (0.25-0.46) | 0 | (0-0) | 0 | (0-0.041) | 3.2810E−03 | 1.000E+00 |
| Free Fatty Acid (16:1) (palmitoleic acid) | 0.42 | (0.31-0.53) | 0.026 | (0-0.066) | 0.015 | (0-0.108) | 7.7932E−02 | 1.000E+00 |
| Methylpyrazine | 0.41 | (0.3-0.51) | 0.026 | (0-0.079) | 0 | (0-0.015) | 4.1891E−02 | 1.000E+00 |
| (R)-MALATE | 0.44 | (0.33-0.54) | 0 | (0-0) | 0.015 | (0-0.116) | 1.1697E−01 | 1.000E+00 |
| 1-HYDROXY-2-NAPHTHOATE | 0.46 | (0.36-0.57) | 0 | (0-0) | 0.015 | (0-0.086) | 2.3642E−01 | 1.000E+00 |
| Free Fatty Acid (18:3) (linolenic acid) | 0.42 | (0.31-0.52) | 0 | (0-0.105) | 0.015 | (0-0.079) | 5.5626E−02 | 1.000E+00 |
| TRANS-ACONITATE | 0.4 | (0.3-0.51) | 0.026 | (0-0.105) | 0.037 | (0.011-0.09) | 3.7170E−02 | 1.000E+00 |
| (R.R)-TARTARIC ACID | 0.51 | (0.41-0.62) | 0 | (0-0) | 0 | (0-0.101) | 3.9167E−01 | 1.000E+00 |
| PIPECOLATE | 0.45 | (0.35-0.55) | 0 | (0-0) | 0.022 | (0-0.067) | 1.6631E−01 | 1.000E+00 |
| Choline | 0.39 | (0.29-0.49) | 0 | (0-0) | 0.015 | (0-0.041) | 2.0716E−02 | 1.000E+00 |
| PHYLLOQUINONE | 0.37 | (0.27-0.47) | 0.053 | (0-0.132) | 0.015 | (0-0.052) | 7.7744E−03 | 1.000E+00 |
| niacinamide | 0.41 | (0.3-0.51) | 0 | (0-0) | 0.007 | (0-0.052) | 3.8384E−02 | 1.000E+00 |
| Lysophosphatidylcholine(26:0) | 0.44 | (0.34-0.54) | 0 | (0-0.066) | 0.052 | (0.011-0.12) | 1.2666E−01 | 1.000E+00 |
| CREATINE | 0.38 | (0.27-0.49) | 0.026 | (0-0.079) | 0 | (0-0.045) | 1.1441E−02 | 1.000E+00 |
| ESTRADIOL-17ALPHA | 0.4 | (0.3-0.51) | 0 | (0-0) | 0 | (0-0.094) | 3.3715E−02 | 1.000E+00 |
| L-LYSINE | 0.44 | (0.34-0.55) | 0 | (0-0) | 0.022 | (0-0.06) | 1.4182E−01 | 1.000E+00 |
| INOSINE_in source | 0.48 | (0.37-0.58) | 0 | (0-0.026) | 0.015 | (0-0.067) | 3.2029E−01 | 1.000E+00 |
| 3-METHYLHISTAMINE_split peak | 0.36 | (0.26-0.46) | 0 | (0-0) | 0.015 | (0-0.06) | 4.4141E−03 | 1.000E+00 |
| Lysophosphatidylcholine(20:5) | 0.42 | (0.32-0.52) | 0 | (0-0.079) | 0.045 | (0.015-0.104) | 5.6042E−02 | 1.000E+00 |
| Prostaglandin A1 | 0.32 | (0.23-0.41) | 0.026 | (0-0.079) | 0.015 | (0-0.075) | 3.3558E−04 | 1.594E−01 |
| THIAMINE MONOPHOSPHATE | 0.38 | (0.28-0.47) | 0 | (0-0.066) | 0.022 | (0-0.086) | 1.1331E−02 | 1.000E+00 |
| CATECHOL | 0.38 | (0.28-0.48) | 0 | (0-0) | 0.022 | (0-0.067) | 1.3979E−02 | 1.000E+00 |
| 3-cis-Hydroxy-b,e-Caroten-3′-one | 0.46 | (0.35-0.57) | 0 | (0-0) | 0.022 | (0-0.049) | 2.3415E−01 | 1.000E+00 |
| Phosphatidylethanolamine(36:4) | 0.43 | (0.33-0.53) | 0 | (0-0.093) | 0.022 | (0-0.079) | 9.4124E−02 | 1.000E+00 |
| Alpha-N-Phenylacetyl-L-glutamine | 0.43 | (0.33-0.53) | 0 | (0-0) | 0 | (0-0.022) | 9.4124E−02 | 1.000E+00 |
| Putative_Creatine riboside | 0.43 | (0.32-0.54) | 0 | (0-0.014) | 0 | (0-0.06) | 8.9860E−02 | 1.000E+00 |
| Testosterone | 0.42 | (0.32-0.52) | 0 | (0-0) | 0.03 | (0.007-0.067) | 6.7727E−02 | 1.000E+00 |
| 1-Methylhistidine | 0.35 | (0.25-0.45) | 0 | (0-0) | 0.015 | (0-0.053) | 2.2034E−03 | 1.000E+00 |
| XANTHURENIC ACID | 0.52 | (0.41-0.63) | 0 | (0-0.066) | 0.06 | (0.022-0.116) | 3.6086E−01 | 1.000E+00 |
| cis-Quinceoxepane [M − H2O + H] + | 0.42 | (0.32-0.53) | 0 | (0-0.105) | 0 | (0-0.082) | 7.8471E−02 | 1.000E+00 |
| L-NORVALINE | 0.45 | (0.34-0.56) | 0 | (0-0.066) | 0.015 | (0-0.101) | 1.7473E−01 | 1.000E+00 |
| HOMOCYSTINE | 0.35 | (0.25-0.44) | 0.026 | (0-0.079) | 0.03 | (0.007-0.09) | 1.8490E−03 | 8.783E−01 |
| 4-HYDROXY-L-PHENYLGLYCINE | 0.4 | (0.3-0.5) | 0 | (0-0) | 0 | (0-0.041) | 3.1824E−02 | 1.000E+00 |
| URIDINE 5′-TRIPHOSPHATE | 0.39 | (0.29-0.49) | 0 | (0-0.04) | 0 | (0-0.135) | 1.5636E−02 | 1.000E+00 |
| Acylcarnitine(C8:0) | 0.39 | (0.29-0.49) | 0 | (0-0) | 0.03 | (0-0.082) | 1.5636E−02 | 1.000E+00 |
| 5-hydroxyindole-3-acetic acid | 0.39 | (0.29-0.49) | 0.026 | (0-0.105) | 0.03 | (0-0.104) | 1.8773E−02 | 1.000E+00 |
| NALPHA-ACETYL-L-LYSINE | 0.37 | (0.27-0.47) | 0 | (0-0.066) | 0.03 | (0.007-0.068) | 6.4636E−03 | 1.000E+00 |
| PICOLINIC ACID | 0.37 | (0.26-0.47) | 0.079 | (0-0.158) | 0 | (0-0.06) | 5.6424E−03 | 1.000E+00 |
| SUCCINATE | 0.32 | (0.22-0.41) | 0 | (0-0) | 0 | (0-0.041) | 3.0522E−04 | 1.450E−01 |
| Taurocholic Acid (TCA) | 0.39 | (0.3-0.48) | 0 | (0-0) | 0.007 | (0-0.06) | 1.8604E−02 | 1.000E+00 |
| Lysophosphatidylcholine(22:6) | 0.39 | (0.28-0.5) | 0 | (0-0.053) | 0.022 | (0-0.064) | 1.8106E−02 | 1.000E+00 |
| Glycerophosphocholine | 0.41 | (0.3-0.51) | 0 | (0-0.04) | 0.022 | (0-0.071) | 3.7471E−02 | 1.000E+00 |
| Sphingomyelin(32:2) | 0.43 | (0.33-0.54) | 0 | (0-0.053) | 0.045 | (0.007-0.116) | 1.0112E−01 | 1.000E+00 |
| cis-Quinceoxepane | 0.41 | (0.3-0.51) | 0 | (0-0.066) | 0.007 | (0-0.064) | 3.7773E−02 | 1.000E+00 |
| LL-2.6-DIAMINOHEPTANEDIOATE | 0.4 | (0.3-0.5) | 0 | (0-0) | 0.007 | (0-0.064) | 2.8067E−02 | 1.000E+00 |
| ADENINE | 0.33 | (0.23-0.44) | 0 | (0-0.079) | 0.015 | (0-0.049) | 8.3688E−04 | 3.975E−01 |
| L-carnitine | 0.35 | (0.25-0.45) | 0 | (0-0.04) | 0.015 | (0-0.045) | 2.0310E−03 | 9.647E−01 |
| 3-SULFINO-L-ALANINE | 0.34 | (0.24-0.45) | 0 | (0-0.053) | 0 | (0-0.022) | 1.4053E−03 | 6.675E−01 |
| Dodecanedioylcarnitine | 0.39 | (0.29-0.49) | 0 | (0-0) | 0 | (0-0.052) | 1.8604E−02 | 1.000E+00 |
| CYSTEINEGLUTATHIONE | 0.38 | (0.28-0.48) | 0 | (0-0.026) | 0.034 | (0.007-0.075) | 1.3334E−02 | 1.000E+00 |
| DISSULFIDE_IN SOURCE FRAGMENT | | | | | | | | |
| OF GSSG | | | | | | | | |
| Lysophosphatidylcholine(17:1) | 0.41 | (0.3-0.52) | 0 | (0-0.079) | 0.007 | (0-0.041) | 4.3569E−02 | 1.000E+00 |
| Chavicol O-beta-glucopyranoside [M + K]+ | 0.36 | (0.27-0.46) | 0 | (0-0) | 0.015 | (0-0.09) | 4.3665E−03 | 1.000E+00 |
| 4-HYDROXY-L-PROLINE | 0.32 | (0.22-0.42) | 0.026 | (0-0.079) | 0.037 | (0.007-0.067) | 3.7875E−04 | 1.799E−01 |
| Coumaric Acid | 0.36 | (0.26-0.47) | 0 | (0-0.053) | 0.022 | (0-0.068) | 5.3522E−03 | 1.000E+00 |
| Plas_Lysophosphatidylcholine(P-18:0/0:0) | 0.38 | (0.28-0.48) | 0 | (0-0) | 0.037 | (0.011-0.079) | 1.2475E−02 | 1.000E+00 |
| or Plas_Lysophosphatidylcholine(O-18:1) | | | | | | | | |
| Ursodeoxycholic acid (UDCA) | 0.47 | (0.37-0.56) | 0 | (0-0) | 0.022 | (0-0.149) | 2.5619E−01 | 1.000E+00 |
| GLUTATHIONE DISSULFIDE | 0.37 | (0.27-0.47) | 0 | (0-0) | 0 | (0-0.037) | 7.2392E−03 | 1.000E+00 |
| 1-linoleoylglycerol | 0.4 | (0.3-0.5) | 0 | (0-0) | 0.007 | (0-0.079) | 3.1824E−02 | 1.000E+00 |
| 1-METHYLNICOTINAMIDE | 0.38 | (0.27-0.49) | 0.026 | (0-0.079) | 0.007 | (0-0.03) | 1.2122E−02 | 1.000E+00 |
| Hyaluronic acid | 0.36 | (0.25-0.47) | 0.026 | (0-0.105) | 0 | (0-0.022) | 4.6088E−03 | 1.000E+00 |
| Phosphatidylcholine(36:5) | 0.4 | (0.3-0.5) | 0 | (0-0.093) | 0.03 | (0.007-0.09) | 2.8067E−02 | 1.000E+00 |
| piperine | 0.39 | (0.28-0.49) | 0 | (0-0) | 0.022 | (0-0.052) | 1.5492E−02 | 1.000E+00 |
| N(PAI)-METHYL-L-HISTIDINE | 0.31 | (0.22-0.41) | 0.026 | (0-0.079) | 0.007 | (0-0.037) | 2.4857E−04 | 1.181E−01 |
| Lysophosphatidylcholine(24:0) | 0.41 | (0.31-0.51) | 0 | (0-0.026) | 0.03 | (0.007-0.052) | 5.0828E−02 | 1.000E+00 |
| D-ORNITHINE | 0.38 | (0.28-0.48) | 0 | (0-0) | 0.015 | (0-0.052) | 1.1552E−02 | 1.000E+00 |
| PlasLysophosphatidylcholine(P-18:0/0:0) | 0.35 | (0.25-0.45) | 0 | (0-0) | 0.015 | (0-0.037) | 2.2034E−03 | 1.000E+00 |
| or PlasLysophosphatidylcholine(O-18:1) | | | | | | | | |
| 2.3-DIHYDROXYBENZOATE | 0.31 | (0.22-0.4) | 0 | (0-0) | 0.022 | (0-0.052) | 1.6813E−04 | 7.986E−02 |
| PARAXANTHINE | 0.4 | (0.3-0.5) | 0 | (0-0) | 0.022 | (0-0.052) | 2.8787E−02 | 1.000E+00 |
| GLUTATHIONE | 0.32 | (0.23-0.42) | 0 | (0-0.014) | 0 | (0-0.045) | 4.2139E−04 | 2.002E−01 |
| LINOLEATE | 0.41 | (0.3-0.53) | 0 | (0-0.093) | 0.015 | (0-0.037) | 5.4394E−02 | 1.000E+00 |
| Lysophosphatidylcholine(17:0) | 0.37 | (0.27-0.46) | 0 | (0-0) | 0.015 | (0-0.037) | 6.2652E−03 | 1.000E+00 |
| Dodecanoic acid | 0.4 | (0.29-0.51) | 0 | (0-0) | 0 | (0-0.041) | 3.3992E−02 | 1.000E+00 |
| N-METHYL-D-ASPARTIC ACID | 0.34 | (0.24-0.43) | 0 | (0-0.079) | 0.007 | (0-0.112) | 1.1566E−03 | 5.494E−01 |
| HISTAMINE | 0.44 | (0.32-0.55) | 0.026 | (0-0.132) | 0.015 | (0-0.056) | 1.1697E−01 | 1.000E+00 |
| PYRIDOXAMINE | 0.35 | (0.26-0.45) | 0 | (0-0) | 0.037 | (0.007-0.075) | 2.6786E−03 | 1.000E+00 |
| Phosphatidylcholine(31:1) | 0.41 | (0.31-0.52) | 0.026 | (0-0.132) | 0.015 | (0-0.071) | 5.1995E−02 | 1.000E+00 |
| L-ASPARAGINE | 0.36 | (0.26-0.45) | 0 | (0-0) | 0.015 | (0-0.052) | 3.2810E−03 | 1.000E+00 |
| CREATINE PHOSPHATE | 0.32 | (0.23-0.42) | 0 | (0-0.053) | 0.045 | (0.015-0.123) | 4.9376E−04 | 2.345E−01 |
| L-ANSERINE | 0.27 | (0.18-0.37) | 0 | (0-0.053) | 0.015 | (0-0.037) | 1.1230E−05 | 5.334E−03 |
| L-N.Gamma.-monomethylarginine | 0.37 | (0.26-0.48) | 0 | (0-0.079) | 0 | (0-0.015) | 6.6674E−03 | 1.000E+00 |
| Lysophosphatidylcholine(22:5) | 0.38 | (0.27-0.5) | 0.026 | (0-0.132) | 0.03 | (0-0.052) | 1.4244E−02 | 1.000E+00 |
| BENZYL ALCOHOL | 0.34 | (0.25-0.43) | 0 | (0-0.026) | 0.037 | (0.015-0.138) | 1.3718E−03 | 6.516E−01 |
| Lysophosphatidylethanolamine(20:4) | 0.4 | (0.28-0.52) | 0 | (0-0.053) | 0.015 | (0-0.052) | 3.0274E−02 | 1.000E+00 |
| Lysophosphatidylcholine(20:4) | 0.35 | (0.24-0.45) | 0 | (0-0.053) | 0.022 | (0.004-0.064) | 2.1033E−03 | 9.991E−01 |
| PlasPhosphatidylethanolamine(o-38:5) or | 0.35 | (0.25-0.45) | 0 | (0-0) | 0.022 | (0.004-0.056) | 2.8351E−03 | 1.000E+00 |
| PlasPhosphatidylethanolamine(p-38:4) | | | | | | | | |
| D-SACCHARIC ACID | 0.33 | (0.24-0.42) | 0 | (0-0) | 0 | (0-0.064) | 8.3688E−04 | 3.975E−01 |
| 2-Arachidonylglycerol | 0.34 | (0.24-0.44) | 0 | (0-0.053) | 0.045 | (0.015-0.082) | 1.1285E−03 | 5.360E−01 |
| TMAO | 0.32 | (0.24-0.41) | 0 | (0-0) | 0.03 | (0.004-0.056) | 4.6231E−04 | 2.196E−01 |
| Lysophosphatidylcholine(16:0) | 0.32 | (0.22-0.41) | 0 | (0-0.105) | 0.015 | (0-0.064) | 3.3108E−04 | 1.573E−01 |
| PlasLysophosphatidylcholine(P-18:0) | 0.32 | (0.22-0.41) | 0 | (0-0) | 0.015 | (0-0.045) | 3.3108E−04 | 1.573E−01 |
| QUINOLINE | 0.31 | (0.22-0.4) | 0 | (0-0) | 0.015 | (0-0.056) | 1.6813E−04 | 7.986E−02 |
| L-HISTIDINE | 0.36 | (0.26-0.45) | 0 | (0-0.079) | 0.015 | (0-0.045) | 3.2810E−03 | 1.000E+00 |
| Ornithine_insource of citrulline | 0.32 | (0.23-0.42) | 0 | (0-0) | 0.015 | (0-0.052) | 4.4434E−04 | 2.111E−01 |
| [M − H2O + H]+ | | | | | | | | |
| 1-(Hydroxymethyl)-5,5-dimethyl-2,4- | 0.33 | (0.24-0.43) | 0 | (0-0) | 0.007 | (0-0.064) | 7.6595E−04 | 3.638E−01 |
| imidazolidinedione_exogenous | | | | | | | | |
| Chavicol O-beta-glucopyranoside | 0.36 | (0.27-0.46) | 0 | (0-0) | 0.015 | (0-0.041) | 5.5247E−03 | 1.000E+00 |
| Phosphatidylethanolamine(38:3) | 0.34 | (0.24-0.43) | 0 | (0-0) | 0.022 | (0-0.056) | 1.0610E−03 | 5.040E−01 |
| Lysophosphatidylcholine(18:0) | 0.31 | (0.22-0.4) | 0 | (0-0) | 0.015 | (0-0.045) | 1.8557E−04 | 8.815E−02 |
| Lysophosphatidylethanolamine(22:0) | 0.33 | (0.24-0.42) | 0 | (0-0) | 0.015 | (0-0.068) | 6.7418E−04 | 3.202E−01 |
| Lysophosphatidic Acid(18:1) | 0.35 | (0.24-0.45) | 0.026 | (0-0.093) | 0.015 | (0-0.045) | 2.3078E−03 | 1.000E+00 |
| PlasLysophosphatidylcholine(O-16:0/0:0) | 0.33 | (0.23-0.42) | 0 | (0-0) | 0.015 | (0-0.037) | 6.1611E−04 | 2.927E−01 |
| Phosphatidylcholine(36:2) | 0.35 | (0.26-0.45) | 0 | (0-0) | 0.022 | (0-0.093) | 2.6483E−03 | 1.000E+00 |
| Acylcarnitine(C18:1) | 0.31 | (0.21-0.41) | 0 | (0-0.053) | 0.015 | (0-0.045) | 1.9355E−04 | 9.194E−02 |
| Sphingosine | 0.3 | (0.21-0.38) | 0 | (0-0) | 0 | (0-0.049) | 8.2781E−05 | 3.932E−02 |
| Lysophosphatidylethanolamine(18:0) | 0.34 | (0.23-0.45) | 0 | (0-0.079) | 0.015 | (0-0.037) | 1.7224E−03 | 8.181E−01 |
| Lysophosphatidylcholine(16:1) | 0.32 | (0.22-0.43) | 0.026 | (0-0.119) | 0.007 | (0-0.082) | 4.0490E−04 | 1.923E−01 |
| Lysophosphatidic Acid(18:0) | 0.29 | (0.2-0.39) | 0 | (0-0.079) | 0.015 | (0-0.06) | 5.2830E−05 | 2.509E−02 |
| THEOBROMINE | 0.35 | (0.26-0.45) | 0 | (0-0) | 0.056 | (0.022-0.116) | 2.5301E−03 | 1.000E+00 |
| Lysophosphatidylcholine(15:0) | 0.29 | (0.2-0.39) | 0 | (0-0) | 0.015 | (0-0.037) | 4.7495E−05 | 2.256E−02 |
| Lysophosphatidylethanolamine(19:0) | 0.31 | (0.21-0.41) | 0 | (0-0) | 0.015 | (0-0.082) | 1.5886E−04 | 7.546E−02 |
| Ornithine_insource of citrulline | 0.29 | (0.2-0.39) | 0 | (0-0) | 0.015 | (0-0.052) | 4.5367E−05 | 2.155E−02 |
| Lysophosphatidylcholine(18:3) | 0.31 | (0.21-0.41) | 0 | (0-0) | 0.015 | (0-0.037) | 2.0469E−04 | 9.723E−02 |
| PlasLysophosphatidylcholine(P-16:0) | 0.26 | (0.17-0.35) | 0.026 | (0-0.093) | 0.015 | (0-0.037) | 3.3241E−06 | 1.579E−03 |
| L-ORNITHINE | 0.29 | (0.2-0.38) | 0 | (0-0) | 0.015 | (0-0.049) | 3.6577E−05 | 1.737E−02 |
| Lysophosphatidylcholine(20:3) | 0.32 | (0.21-0.43) | 0.053 | (0-0.105) | 0.015 | (0-0.03) | 4.3849E−04 | 2.083E−01 |
| Lysophosphatidylcholine(18:1) | 0.31 | (0.2-0.41) | 0.026 | (0-0.079) | 0.015 | (0-0.037) | 1.4379E−04 | 6.830E−02 |
| Lysophosphatidylethanolamine(18:2) | 0.35 | (0.25-0.45) | 0 | (0-0) | 0.007 | (0-0.034) | 2.3346E−03 | 1.000E+00 |
| Lysophosphatidylcholine(14:0) | 0.28 | (0.19-0.38) | 0.053 | (0-0.132) | 0.007 | (0-0.037) | 2.6352E−05 | 1.252E−02 |
| Lysophosphatidylethanolamine(18:1) | 0.29 | (0.2-0.38) | 0 | (0-0.053) | 0.015 | (0-0.037) | 3.9513E−05 | 1.877E−02 |
| Lysophosphatidylcholine(20:2) | 0.28 | (0.18-0.38) | 0.026 | (0-0.079) | 0.015 | (0-0.037) | 1.8577E−05 | 8.824E−03 |
| Lysophosphatidylcholine(18:2) | 0.3 | (0.2-0.4) | 0 | (0-0) | 0.015 | (0-0.045) | 8.0368E−05 | 3.817E−02 |

TABLE 5
Individual predictive performance of selected metabolites in the Training Set.

| | | Sensitivity | Specificity |
|---|---|---|---|
| | | (95% CI) @ | (95% CI) @ |
| Features | AUC (95% CI) | 99% specificity | 99% sensitivity |
| Diacetylspermine (DAS) | 0.82 (0.76-0.88) | 0.28 (0.05-0.50) | 0.07 (0.03-0.34) |
| N-acetylneuraminate (NANA) | 0.65 (0.58-0.73) | 0.03 (0.00-0.08) | 0.05 (0.01-0.15) |
| N-acetyl-mannosamine (NAcMan) | 0.58 (0.50-0.65) | 0.05 (0.01-0.12) | 0.04 (0.00-0.17) |
| N-acetyl-lactosamine (NAcLac) | 0.55 (0.48-0.63) | 0.03 (0.00-0.07) | 0.04 (0.01-0.08) |
| Diacetylspermidine (DiAcSpmd) | 0.67 (0.60-0.74) | 0.03 (0.00-0.18) | 0.05 (0.01-0.10) |
| N−(3-acetamidopropyl)pyrrolidin-2-one (N3AP) | 0.56 (0.49-0.64) | 0.05 (0.00-0.13) | 0.03 (0.00-0.08) |
| Hydroxyisobutyrate (HBA) | 0.71 (0.64-0.77) | 0.08 (0.03-0.20) | 0.16 (0.00-0.25) |

### Example 5: Model Building and Testing

An optimal combination rule that incorporated the seven metabolites for distinguishing early-stage OvCa from benign disease was developed. For model building, seven different machine learning algorithms were tested. Of these, a deep learning model (DLM) with 3 hidden layers and 3 nodes in each layer achieved the highest predictive performance and was used to establish the 7-marker metabolite panel (7MetP), which yielded an AUC of 0.75 (95% CI: 0.66-0.85) for differentiating early-stage OvCa cases from benign disease (Tables 6-8). When stratifying OvCa cases into serous and non-serous, the 7MetP had respective AUCs of 0.85 (95% CI: 0.79-0.91) and 0.80 (95% CI: 0.71-0.89) (Table 9).

Validation of the 7MetP using fixed parameters was performed in an independent Test Set from MD Anderson Cancer Center (MDACC) that consisted of 118 OvCa cases (20 early stage and 98 late stage) and 56 individuals with BPM. The 7MetP yielded an AUC of 0.88 (95% CI: 0.82-0.93) for distinguishing all OvCa cases from individuals with BPM (Table 7), and an AUC of 0.86 (95% CI: 0.76-0.95) for early-stage OvCa (FIG. 1; Table 7).

TABLE 6
Performance of different learning algorithms for differentiating early-stage
OvCa cases from BPM in the training set using 5-fold cross validation.

| | | | Log | | Mean per | |
|---|---|---|---|---|---|---|
| Model | Hyper parameters | AUC¹ | Loss | AUCpr² | class error | RMSE³ |
| Deep learning model | Activation: MaxoutWithDropout, | 0.753 | 0.354 | 0.556 | 0.222 | 0.303 |
| | hidden layers: [3, 3, 3] | | | | | |
| Deep learning model | Activation: Tanh, hidden layers: [1, 1] | 0.740 | 0.362 | 0.529 | 0.233 | 0.322 |
| StackedEnsemble | Ensemble models: GLM, Deep | 0.713 | 0.387 | 0.484 | 0.205 | 0.332 |
| | Learning, Random Forest, Gradient | | | | | |
| | Boost Method | | | | | |
| Deep learning model | Activation: Tanh, hidden layers: [2, 2] | 0.711 | 0.377 | 0.519 | 0.237 | 0.325 |
| Lasso Regression | Lambda = 0.2, 5 features selected | 0.709 | 0.506 | 0.438 | 0.202 | 0.376 |
| StackedEnsemble | Ensemble models (best of each | 0.692 | 0.399 | 0.459 | 0.228 | 0.336 |
| | family): GLM, Deep Learning, | | | | | |
| | Random Forest, Gradient Boost | | | | | |
| | Method | | | | | |
| GLM | Family: binomial | 0.687 | 0.532 | 0.447 | 0.241 | 0.364 |
| Extremely Randomized | — | 0.681 | 0.577 | 0.359 | 0.224 | 0.351 |
| Trees (XRT) | | | | | | |
| Distributed Random | — | 0.679 | 0.746 | 0.355 | 0.216 | 0.354 |
| Forest (DRF) | | | | | | |
| Gradient Boosting | Number of tree: 50, | 0.668 | 0.516 | 0.357 | 0.234 | 0.372 |
| Method | Maximum depth: 6 | | | | | |

¹AUC: Area under the ROC Curve
²AUCpr: Area under the precision recall curve
³RMSE: Root-mean-square deviation

TABLE 7
Performance of the 7-marker metabolite panel (7MetP) for distinguishing
OvCa cases from individuals with BPM in the Training Set, the independent
Test Set, and the combined Training + Testing Specimen Set.

| | Training Set | | Testing Set | | Combined Dataset | |
|---|---|---|---|---|---|---|
| | Early-Stage | All Stage | Early-Stage | All Stage | Early-Stage | All Stage |
| | (n = 39) | (n = 101) | (n = 20) | (n = 118) | (n = 59) | (n = 219) |
| AUC | 0.75 (0.66- | 0.83 (0.78- | 0.86 (0.76- | 0.88 (0.82- | 0.81 (0.76- | 0.85 (0.81- |
| | 0.85) | 0.89) | 0.95) | 0.93) | 0.86) | 0.88) |
| Sensitivity at 99% | 0.21 (0.08- | 0.41 (0.17- | 0.15 (0.00- | 0.40 (0.31- | 0.20 (0.11- | 0.39 (0.20- |
| specificity | 0.40) | 0.50) | 0.35) | 0.62) | 0.28) | 0.47) |
| Sensitivity at 95% | 0.24 (0.11- | 0.47 (0.35- | 0.30 (0.05- | 0.51 (0.33- | 0.25 (0.15- | 0.46 (0.38- |
| specificity | 0.47) | 0.66) | 0.65) | 0.75) | 0.44) | 0.62) |
| Specificity at 99% | 0.54 (0.40- | 0.16 (0.06- | 0.36 (0.24- | 0.11 (0.02- | 0.13 (0.08- | 0.12 (0.05- |
| sensitivity | 0.78) | 0.30) | 0.80) | 0.42) | 0.35) | 0.28) |
| Specificity at 95% | 0.10 (0.05- | 0.29 (0.14- | 0.55 (0.27- | 0.40 (0.07- | 0.32 (0.19- | 0.31 (0.20- |
| sensitivity | 0.28) | 0.47) | 0.87) | 0.62) | 0.50) | 0.44) |

TABLE 8
Stability check of the deep learning model (DLM) in the Training Set.

| | Scenario #1 | Scenario #2 | Scenario #3 | Scenario #4 | Scenario #5 |
|---|---|---|---|---|---|
| | Early-stage | All stage | 500 random | 173 random | Late-stage |
| | OvCa cases | OvCa cases | samples with | samples with | OvCa cases |
| | and BPM | and BPM | replacement | replacement | and BPM |
| N0 (# of BPM) | 134 | 134 | 406 | 143 | 134 |
| N1 (# of cases) | 39 | 100 | 94 | 30 | 61 |
| AUC | 0.75 | 0.83 | 0.76 | 0.76 | 0.88 |
| | (0.66-0.85) | (0.78-0.89) | (0.70-0.82) | (0.68-0.84) | (0.83-0.94) |
| Sensitivity at | 0.26 | 0.46 | 0.35 | 0.20 | 0.61 |
| 95% specificity | (0.11-0.47) | (0.36-0.64) | (0.17-0.56) | (0.10-0.45) | (0.47-0.78) |
| Specificity at | 0.16 | 0.29 | 0.18 | 0.34 | 0.33 |
| 95% sensitivity | (0.05-0.47) | (0.16-0.49) | (0.12-0.28) | (0.14-0.50) | (0.22-0.68) |

TABLE 9
Predictive performance of the 7MetP for distinguishing OvCa cases stratified into
serous and non-serous from BPM in the Training Set.

| | Serous carcinoma | | Non-serous carcinoma | |
|---|---|---|---|---|
| | Early-Stage | All Stage | Early-Stage | All Stage |
| | (n = 13) | (n = 69) | (n = 25) | (n = 31) |
| AUC | 0.69 (0.52-0.86) | 0.85 (0.79-0.91) | 0.79 (0.68-0.89) | 0.80 (0.71-0.89) |
| Sensitivity at 99% | 0.23 (0.00-0.46) | 0.48 (0.17-0.61) | 0.20 (0.04-0.36) | 0.23 (0.10-0.39) |
| specificity | | | | |
| Sensitivity at 95% | 0.23 (0.00-0.54) | 0.55 (0.39-0.71) | 0.24 (0.08-0.44) | 0.29 (0.13-0.55) |
| specificity | | | | |
| Specificity at 99% | 0.10 (0.05-0.48) | 0.11 (0.05-0.28) | 0.19 (0.11-0.44) | 0.19 (0.11-0.40) |
| sensitivity | | | | |
| Specificity at 95% | 0.11 (0.06-0.51) | 0.26 (0.14-0.51) | 0.33 (0.13-0.59) | 0.30 (0.12-0.52) |
| sensitivity | | | | |

Contributions of the Metabolite Panel with the ROMA Algorithm

The 7MetP was next assessed to gauge if it improved upon the predictive performance of the ROMA algorithm. Using model scores derived from the 7MetP and the ROMA algorithm, a logistic regression model for distinguishing early-stage OvCa from BPM was developed in the Training Set and its performance evaluated in the Test Set. The combined 7MetP+ROMA yielded an AUC of 0.93 (95% Cl: 0.86-1.00) for early-stage OvCa in the Test Set, whereas ROMA alone had an AUC of 0.91 (95% Cl: 0.84-0.98) (likelihood ratio test p: 0.03). Compared to ROMA, the combined 7MetP+ROMA yielded improvements in the PPV by 21.0% (1-sided p<0.001) and specificity by 14.0% (1-sided p<0.001) for early stage OvCa (Table 10). When considering all OvCa cases, the combined 7MetP+ROMA model yielded an AUC of 0.97 (95% CI: 0.94-0.99) in the Test Set (Table 11).

TABLE 10
Performance estimates of ROMA and the combined 7MetP+ROMA model for
early-stage OvCa the Training Set and the independent Testing Set.

| | | ROMA | ROMA + 7MetP | Difference | P |
|---|---|---|---|---|---|
| | Training Set | | | | |
| | AUC (95% CI) | 0.81 (0.77-0.85) | 0.84 (0.80-0.88) | 0.03 (0.01 to 0.06) | <.001 |
| At 11.4% risk | Sensitivity | 0.68 (0.60-0.77) | 0.68 (0.59-0.77) | 0.00 (−0.06 to 0.07) | .49 |
| threshold for | | | | | |
| premenopausal | Specificity | 0.79 (0.75-0.83) | 0.88 (0.85-0.91) | 0.09 (0.06 to 0.12) | <.001 |
| and 29.9% for | | | | | |
| postmenopausal | PPV | 0.48 (0.40-0.55) | 0.62 (0.54-0.69) | 0.14 (0.08 to 0.20) | <.001 |
| (same risk as | | | | | |
| ROMA) | NPV | 0.90 (0.87-0.93) | 0.91 (0.88-0.94) | 0.01 (−0.01 to 0.03) | .14 |
| | Test Set | | | | |
| | AUC (95% CI) | 0.91 (0.84-0.98) | 0.93 (0.86-1.00) | 0.02 (−0.01 to 0.04) | .03 |
| At 11.4% risk | Sensitivity | 0.90 (0.83-0.97) | 0.90 (0.81-0.97) | 0.00 (−0.09 to 0.09) | .472 |
| threshold for | | | | | |
| premenopausal | Specificity | 0.77 (0.70-0.83) | 0.91 (0.86-0.95) | 0.14 (0.09 to 0.20) | <.001 |
| and 29.9% for | | | | | |
| postmenopausal | PPV | 0.58 (0.48-0.68) | 0.79 (0.67-0.87) | 0.21 (0.13 to 0.28) | <.001 |
| (same risk as | | | | | |
| ROMA) | NPV | 0.96 (0.92-0.99) | 0.96 (0.93-0.99) | 0.01 (−0.03 to 0.04) | .365 |

PPV: Positive predictive value
NPV: Negative predictive value
P: P-values for likelihood ratio tests

TABLE 11
Performance estimates of ROMA and the combined 7MetP+ROMA model for all
OvCa in the Training Set and the independent Testing Set.

| | | ROMA | ROMA + 7MetP | Difference | P |
|---|---|---|---|---|---|
| | Training Set | | | | |
| | AUC (95% CI) | 0.91 (0.89-0.93) | 0.93 (0.91-0.94) | 0.01 (0.00 to 0.03) | <.001 |
| At 11.4% risk | Sensitivity | 0.87 (0.83-0.91) | 0.86 (0.82-0.90) | −0.01 (−0.04 to 0.02) | .22 |
| threshold for | | | | | |
| premenopausal | Specificity | 0.79 (0.75-0.83) | 0.88 (0.85-0.91) | 0.09 (0.06 to 0.12) | <.001 |
| and 29.9% for | | | | | |
| postmenopausal | PPV | 0.76 (0.71-0.80) | 0.84 (0.81-0.89) | 0.09 (0.05 to 0.12) | <.001 |
| (same risk as | | | | | |
| ROMA) | NPV | 0.89 (0.86-0.92) | 0.89 (0.86-0.92) | 0.00 (−0.02 to 0.02) | .36 |
| | Test Set | | | | |
| | AUC (95% CI) | 0.96 (0.94-0.99) | 0.97 (0.94-0.99) | 0.01 (0.00 to 0.01) | .06 |
| At 11.4% risk | Sensitivity | 0.96 (0.93-0.98) | 0.93 (0.90-0.96) | −0.03 (−0.05 to −0.01) | .008 |
| threshold for | | | | | |
| premenopausal | Specificity | 0.76 (0.70-0.83) | 0.91 (0.86-0.95) | 0.15 (0.10 to 0.20) | <.001 |
| and 29.9% for | | | | | |
| postmenopausal | PPV | 0.89 (0.86-0.93) | 0.96 (0.93-0.97) | 0.06 (0.04 to 0.09) | <.001 |
| (same risk as | | | | | |
| ROMA) | NPV | 0.89 (0.84-0.94) | 0.86 (0.81-0.91) | −0.03 (−0.08 to 0.01) | .07 |

PPV: Positive predictive value
NPV: Negative predictive value
P: P-values for likelihood ratio tests

Performance of the Metabolite Panel Alone and in Combination with ROMA in the Combined Training Set and Test Sets.

The predictive performance of the 7MetP alone and in combination with ROMA was further evaluated in the entire specimen set (n=219 OvCa cases (59 early stage and 160 late stage and 190 BPM)). The 7MetP had an AUC of 0.85 (95% CI: 0.81-0.88) for distinguishing all OvCa cases from individuals with BPM and an AUC of 0.81 (95% CI: 0.76-0.86) for early-stage OvCa (Table 7). The combined 7MetP+ROMA model had a resultant AUC of 0.87 (95% CI: 0.85-0.93) for early-stage OvCa, which was markedly improved compared to ROMA alone (AUC: 0.84 (95% CI: 0.81-0.90); likelihood ratio test p-value: <0.001) (Tables 12 and 13). Importantly, compared to ROMA alone, the 7MetP+ROMA model yielded a statistically significantly (1-sided P<0.001) higher PPV (0.68 vs 0.52) and specificity (0.89 versus 0.78) for early-stage OvCa (Table 5).

TABLE 12
Performance estimates of ROMA and the combined 7MetP+ROMA model for
early-stage OvCa in the combined Specimen Set.

| | Entire Set | | | | |
|---|---|---|---|---|---|
| | | ROMA | ROMA + 7MetP | Difference | P |
| | AUC (95% CI) | 0.84 (0.79-0.90) | 0.87 (0.82-0.93) | 0.03 (0.01 to 0.04) | <.001 |
| At 11.4% risk | Sensitivity | 0.76 (0.69-0.82) | 0.76 (0.70-0.82) | 0.00 (−0.04 to 0.05) | .464 |
| threshold for | | | | | |
| premenopausal | Specificity | 0.78 (0.75-0.81) | 0.89 (0.86-0.92) | 0.11 (0.08 to 0.14) | <.001 |
| and 29.9% for | | | | | |
| postmenopausal | PPV | 0.52 (0.45-0.58) | 0.68 (0.61-0.75) | 0.16 (0.11 to 0.21) | <.001 |
| (same risk as | | | | | |
| ROMA) | NPV | 0.91 (0.89-0.94) | 0.92 (0.90-0.94) | 0.01 (−0.01 to 0.02) | .091 |

PPV: Positive predictive value
NPV: Negative predictive value
P: P-values for likelihood ratio tests

TABLE 13
Performance estimates of ROMA and the combined 7MetP+ROMA model for all
OvCa in the combined specimen set.
All OvCa (n = 219) vs BPM (N = 190)

| | | ROMA | ROMA + 7MetP | Difference | P |
|---|---|---|---|---|---|
| | AUC (95% CI) | 0.94 (0.92-0.95) | 0.94 (0.93-0.96) | 0.01 (0.00 to 0.01) | .001 |
| At 11.4% risk | Sensitivity | 0.92 (0.89-0.94) | 0.90 (0.87-0.92) | −0.02 (−0.04 to 0.00) | .01 |
| threshold for | | | | | |
| premenopausal and | Specificity | 0.78 (0.75-0.82) | 0.89 (0.86-0.91) | 0.11 (0.08 to 0.14) | <.001 |
| 29.9% for | | | | | |
| postmenopausal (same | PPV | 0.83 (0.80-0.86) | 0.90 (0.88-0.92) | 0.07 (0.05 to 0.10) | <.001 |
| risk as ROMA) | NPV | 0.89 (0.86-0.92) | 0.88 (0.86-0.91) | −0.01 (−0.03 to 0.01) | .21 |

All references, patents or applications, U.S. or foreign, cited in the application are hereby incorporated by reference as if written herein in their entireties. Where any inconsistencies arise, material literally disclosed herein controls.

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A method of treatment of ovarian cancer in a patient having elevated levels of diacetylspermine, diacetylspermidine, N-(3-acetamidopropyl)pyrrolidin-2-one, N-acetylneuraminate, N-acetyl-mannosamine, N-acetyl-lactosamine, and hydroxyisobutyric acid, and optionally, elevated levels of Human epididymal protein 4 (HE4) and Mucin 16 (CA125), wherein the elevated levels classify the patient as having ovarian cancer, comprising administering a therapeutically effective amount of a treatment for ovarian cancer to the patient.

2. A method of treatment of ovarian cancer, comprising:
a) identifying a patient having elevated levels of diacetylspermine, diacetylspermidine, N-(3-acetamidopropyl)pyrrolidin-2-one, N-acetylneuraminate, N-acetyl-mannosamine, N-acetyl-lactosamine, and hydroxyisobutyric acid, and optionally, elevated levels of Human epididymal protein 4 (HE4) and Mucin 16 (CA125), wherein the elevated levels classify the patient as having ovarian cancer; and
b) administering a therapeutically effective amount of a treatment for ovarian cancer to the patient.

3. A method of distinguishing ovarian cancer from a benign pelvic mass (BPM) in a subject, comprising, in a biological sample obtained from the subject:
a) measuring the levels of diacetylspermine, diacetylspermidine, N-(3-acetamidopropyl)pyrrolidin-2-one, N-acetylneuraminate, N-acetyl-mannosamine, N-acetyl-lactosamine, and hydroxyisobutyric acid, and optionally, the levels of Human epididymal protein 4 (HE4) and Mucin 16 (CA125), in the biological sample; and
b) classifying the subject as having either ovarian cancer or a BPM based on said measured levels.

4. A method of determining the risk of a subject for harboring ovarian cancer, comprising, in a biological sample obtained from the subject:
a) measuring the levels of diacetylspermine, diacetylspermidine, N-(3-acetamidopropyl)pyrrolidin-2-one, N-acetylneuraminate, N-acetyl-mannosamine, N-acetyl-lactosamine, and hydroxyisobutyric acid, and optionally, the levels of Human epididymal protein 4 (HE4) and Mucin 16 (CA125), in the biological sample; and
b) classifying the subject as being at risk of harboring ovarian cancer or not being at risk of harboring ovarian cancer based on said measured levels.

5. A method of producing a risk profile of a subject for harboring ovarian cancer, comprising, in a biological sample obtained from the subject:
a) measuring the levels of diacetylspermine, diacetylspermidine, N-(3-acetamidopropyl)pyrrolidin-2-one, N-acetylneuraminate, N-acetyl-mannosamine, N-acetyl-lactosamine, and hydroxyisobutyric acid, and optionally, the levels of Human epididymal protein 4 (HE4) and Mucin 16 (CA125), in the biological sample; and
b) classifying the subject as being at risk of harboring ovarian cancer or not being at risk of harboring ovarian cancer based on said measured levels.

6. A method of risk stratification for a patient at risk of harboring ovarian cancer, comprising, in a biological sample obtained from the patient:
(a) measuring the levels of diacetylspermine, diacetylspermidine, N-(3-acetamidopropyl)pyrrolidin-2-one, N-acetylneuraminate, N-acetyl-mannosamine, N-acetyl-lactosamine, and hydroxyisobutyric acid, and optionally, the levels of Human epididymal protein 4 (HE4) and Mucin 16 (CA125), in the biological sample; and
(b) determining, by processor circuitry, the risk score for the patient, wherein the risk score is determined via a scoring function derived from metabolite profiles for biological samples taken from a plurality of individuals that were monitored for ovarian cancer.

7. A method for calculating a patient's biomarker score or risk score of harboring ovarian cancer, comprising, in a biological sample obtained from the patient:
(a) measuring the levels of diacetylspermine, diacetylspermidine, N-(3-acetamidopropyl)pyrrolidin-2-one, N-acetylneuraminate, N-acetyl-mannosamine, N-acetyl-lactosamine, and hydroxyisobutyric acid, and optionally, the levels of Human epididymal protein 4 (HE4) and Mucin 16 (CA125), in the biological sample; and
(b) calculating the biomarker score or risk score using the numerical values of the measured levels in a deep learning model (DLM).

8. The method of claim 7, wherein the DLM comprises an artificial neural network which has three hidden layers and three nodes in each layer.

9. The method of any one of claims 1-7, further comprising measuring the levels of or identifying a patient with elevated levels of Human epididymal protein 4 (HE4) and Mucin 16 (CA125).

10. The method of any one of claims 1-9, wherein the levels of HE4 and CA125 are determined by an immunoassay.

11. The method of claim 10, wherein the levels of HE4 and CA125 are used to calculate a predictive index (PI) for premenopausal women with the equation:
$$PI = - 12.0 + 2.38 * ln [ HE 4 ] + 0 \text{.0626} * ln [ CA 125 ] .$$

12. The method of claim 10, wherein the levels of HE4 and CA125 are used to calculate a predictive index (PI) for postmenopausal women with the equation:
$$PI = - 8.09 + 1.04 * ln [ HE 4 ] + 0.732 * ln [ CA 125 ] .$$

13. The method of either claim 11 or 12, wherein the predictive index (PI) is used to calculate a Risk of Ovarian Malignancy (ROMA) score with the equation:
$$ROMA score ( \% ) = \frac{exp ( PI )}{1 + exp ( PI )} * 100$$

14. The method of claim 13, wherein a combined model score is calculated using a logistic regression with the ROMA score and the biomarker score.

15. The method of any one of claims 1-14, wherein the ovarian cancer is early stage (e.g., stage I or II).

16. The method of any one of claims 1-14, wherein the ovarian cancer is advanced (e.g., stage III or IV).

17. The method of any one of claims 1-7, wherein the levels of diacetylspermine, diacetylspermidine, N-(3-acetamidopropyl)pyrrolidin-2-one, N-acetylneuraminate, N-acetyl-mannosamine, N-acetyl-lactosamine, and hydroxyisobutyric acid, and optionally, HE4 and CA125, are elevated relative to a reference patient or group that does not have ovarian cancer.

18. The method of any one of claims 1-7, wherein the levels of diacetylspermine, diacetylspermidine, N-(3-acetamidopropyl)pyrrolidin-2-one, N-acetylneuraminate, N-acetyl-mannosamine, N-acetyl-lactosamine, and hydroxyisobutyric acid, and optionally, HE4 and CA125, are elevated relative to a reference patient or group that has a benign pelvic mass (BPM).

19. The method of any one of claims 1-7, wherein the subject presents with a pelvic mass.

20. The method of any preceding claim, wherein each of diacetylspermine, diacetylspermidine, N-(3-acetamidopropyl)pyrrolidin-2-one, N-acetylneuraminate, N-acetyl-mannosamine, N-acetyl-lactosamine, and hydroxyisobutyric acid, and optionally, HE4 and CA125, generates a detectable signal.

21. The method of claim 20, wherein the detectable signals are detectable by a spectrometric method.

22. The method of claim 21, wherein the spectrometric method is chosen from UV-visible spectroscopy, mass spectroscopy, nuclear magnetic resonance (NMR) spectroscopy, proton NMR spectroscopy, nuclear magnetic resonance (NMR) spectrometry, gas chromatography, mass spectrometry (GC-MS), liquid chromatography-mass spectrometry (LC-MS), correlation spectroscopy (COSY), nuclear Overhauser effect spectroscopy (NOESY), rotating-frame nuclear Overhauser effect spectroscopy (ROESY), time-of-flight LC-MS (LC-TOF-MS), liquid chromatography-tandem mass spectrometry (LC-MS/MS), and capillary electrophoresis-mass spectrometry.

23. The method of claim 22, wherein the spectrometric method is mass spectrometry.

24. The method of claim 23, wherein the mass spectrometry is LC-TOF-MS.

25. The method of claim 1, 2, 15, or 16 wherein the treatment is chosen from surgery, chemotherapy, immunotherapy, radiation therapy, targeted therapy, or a combination thereof.

26. The method of any one of claims 1-7, wherein the measured levels are used to calculate a biomarker score or risk profile based on sensitivity and specificity values that corresponds to the risk of the subject for harboring ovarian cancer.

27. The method of claim 26, wherein the sensitivity and specificity values do not differ substantially from the curve in FIG. 1.

28. The method of claim 27, wherein the sensitivity and specificity values differ by less than 10%.

29. The method of claim 28, wherein the sensitivity and specificity values differ by less than 5%.

30. The method of claim 29, wherein the sensitivity and specificity values differ by less than 1%.

31. The method of any one of claims 1-7, wherein the cutoff value comprises an AUC (95% CI) of at least 0.76.

32. The method as recited in any previous claim, further comprising assigning the patient to an appropriate risk group based on the calculated risk score.

33. The method of claim 32, wherein there are at least two risk groups.

34. The method of any one of claims 1-7, wherein the AUC of the method is greater than the AUC for a different biomarker, biomarkers, panel, assay, or algorithm incorporating a combination thereof.

35. The method of claim 34, wherein the AUC is greater than 0.76.

36. The method of claim 35, wherein the AUC is between 0.76 and 0.95.

37. The method of claim 36, wherein the AUC is about 0.88.

38. The method of claim 36, wherein the AUC is about 0.86.

39. The method of claim 35, wherein the AUC is between 0.82 and 0.93

40. The method of claim 39, wherein the AUC is about 0.87.

41. The method of any one of claims 1-7, wherein the positive predictive value (PPV) of the method is greater than the PPV for a different biomarker, biomarkers, panel, assay, or algorithm incorporating a combination thereof.

42. The method of claim 41, wherein the PPV is greater than 0.67.

43. The method of claim 42, wherein the PPV is between 0.67 and 0.87.

44. The method of claim 43, wherein the PPV is about 0.79.

45. The method of any one of claims 34-44, wherein the algorithm is the Risk of Ovarian Malignancy Algorithm (ROMA).

46. The method of any one of claims 34-44, wherein the biomarkers are HE4 and CA125 alone.

47. The method of either claim 45 or 46, wherein the cutoff points of the respective methods are used for classification.

48. The method of either claim 45 or 46, analyzed by the same statistical methods.

49. The method as recited in any previous claim, wherein the levels of diacetylspermine, diacetylspermidine, N-(3-acetamidopropyl)pyrrolidin-2-one, N-acetylneuraminate, N-acetyl-mannosamine, N-acetyl-lactosamine, and hydroxyisobutyric acid are measured against a given threshold value or values.

50. The method of claim 49, wherein the values exceed the threshold value or values and the patient is classified as being at risk of harboring ovarian cancer.

51. The method of claim 50, wherein the values are below the threshold value or values and the patient is classified as being not at risk of harboring ovarian cancer.

52. The method of claim 50, wherein the values are below the threshold value or values and the patient is classified as having a BPM.

53. The method of claim 51, wherein the patient is subsequently designated for further ovarian cancer screening or treatment.

54. The method of claim 53, wherein the screening is chosen from endopic ultrasound, magnetic resonance imaging (MRI), and computed topography (CT) scans.

55. The method of claim 54, wherein the screening is performed annually.

56. The method of claim 54, wherein the screening is performed semi-annually.
